SEQUENCE LISTING (17 sequences)

SEQ ID NO: 1 - protein, 9 aa, Homo sapiens
FMLTQPHSV

SEQ ID NO: 2 - protein, 9 aa, Homo sapiens
YMLTQPHSV

SEQ ID NO: 3 - protein, 9 aa, Homo sapiens
FLLTQPHSV

SEQ ID NO: 4 - protein, 9 aa, Homo sapiens
SVLTQPPSV

SEQ ID NO: 5 - protein, 9 aa, Homo sapiens
SELTQPASV

SEQ ID NO: 6 - protein, 9 aa, Homo sapiens
SELTQPPSV

SEQ ID NO: 7 - protein, 9 aa, Mus musculus
SLFPRLYPV

SEQ ID NO: 8 - protein, 9 aa, Mus musculus
YLFPRLYPV

SEQ ID NO: 9 - protein, 13 aa, Influenza virus
PKYVKQNTLKLAT

SEQ ID NO: 10 - protein, 13 aa, Artificial Sequence
  Description of Artificial Sequence Synthetic peptide
AKXVAAWTLKAAA

SEQ ID NO: 11 - protein, 16 aa, Plasmodium sp.
EKKIAKMEKASSVFNV

SEQ ID NO: 12 - protein, 10 aa, Hepatitis B virus
FFLLTRILTI

SEQ ID NO: 13 - protein, 19 aa, Homo sapiens
DQSIGDLIAEAMDKVGNEG

SEQ ID NO: 14 - protein, 14 aa, Mycobacterium bovis
QVHFQPLPPAVVKL

SEQ ID NO: 15 - protein, 15 aa, Clostridium tetani
QYIKANSKFIGITEL

SEQ ID NO: 16 - protein, 21 aa, Clostridium tetani
FNNFTVSFWLRVPKVSASHLE

SEQ ID NO: 17 - protein, 16 aa, Human immunodeficiency virus
KQIINMWQEVGKAMYA

(19) United States
(12) Patent Application Publication
(10) Pub. No.: US 2009/0028847 A1
(43) Pub. Date: Jan. 29, 2009

(54) MULTIPLE MYELOMA AND AL AMYLOID IMMUNOTHERAPY TARGETING IMMUNOGLOBULIN LIGHT CHAINS AND USES THEREOF

(75) Inventors: David H. Sherr, West Roxbury, MA (US); Amanda Flies, Jamaica Plain, MA (US)

Correspondence Address: RONALD I. EISENSTEIN, 100 SUMMER STREET, NIXON PEABODY LLP, BOSTON, MA, 02110, US

(73) Assignee: TRUSTEES OF BOSTON UNIVERSITY, Boston, MA (US)

(21) Appl. No.: 12/102,691

(22) Filed: Apr. 14, 2008

Related U.S. Application Data
(60) Provisional application No. 60/923,068, filed on Apr. 12, 2007.

(51) Int. Cl. A61K 39/395; A61P 35/00; A61K 39/44; C12Q 1/02

(52) U.S. Cl. 424/130.1; 424/178.1; 424/179.1; 435/29

(57) ABSTRACT

The present invention relates generally to the prevention and treatment of disease states, and more particularly to the treatment and prevention of plasma cell disorders and plasma cell dyscrasias and other malignancies, amyloidosis and amyloid-associated diseases. In particular, the present invention relates to methods and compositions comprising immunogenic peptides for the treatment and prevention of diseases and malignancies, for example plasma cell disorders, plasma cell dyscrasias and amyloidosis or amyloid-associated diseases. The present invention also provides methods for an assay to screen for therapeutic vaccines for plasma cell disorders, plasma cell dyscrasias and amyloidosis or amyloid-associated diseases.

### CROSS REFERENCED APPLICATIONS

This application claims benefit under 35 U.S.C. 119(e) of U.S. Provisional Patent Application Ser. No. 60/923,068 filed on Apr. 12, 2007 the contents of which is incorporated herein in its entity by reference.

### GOVERNMENT SUPPORT

The present application was supported by the National Institutes for Health (N1H) Grant No: 1PO1 HL68705, and the Government of the United States has certain rights thereto.

### FIELD OF THE INVENTION

The present invention relates generally to the prevention and treatment of disease states, and more particularly to the treatment and prevention of plasma cell disorders and plasma cell dyscrasias and other malignancies, amyloidosis and amyloid-associated diseases. The present invention further comprises methods and compositions comprising immunogenic peptides for the treatment and prevention of diseases and malignancies, for example plasma cell disorders, plasma cell dyscrasias and amyloidosis or amyloid-associated diseases, and the methods for an assay for screening therapeutic vaccines thereof.

### BACKGROUND OF THE INVENTION

AL Amyloidosis is characterized by organ destruction due to accumulation of protein fibrils composed of misfolded immunoglobulin light chains produced by bone marrow plasma cells. Amyloidosis is characterized by organ destruction due to accumulation of protein fibrils composed of misfolded immunoglobulin light chains. The plasma cell dyscrasia which underlies the disease results in the over-production of the light chains by a bone marrow plasma cell clone [1].

AL amyloidosis and multiple myeloma. AL (amyloid light chain) amyloidosis is a lethal disorder characterized by the cumulative deposition of clonal plasma cell-derived, fibrillogenic immunoglobulin light chains in vital organs including the kidney (65%), heart (45%), and lung (20%) (Falk et al, 2000), which impair function. It is thought that fibrillogenesis is a function of high Ig light chain serum concentrations, specific biophysical factors that influence protein folding (Obici et al, 2005), and association with as yet undefined nucleating elements such as proteoglycans (Trinkaus-Randall et al, 2005). As with other plasma cell dyscrasias (multiple myeloma, MGUS, Waldenstrom's macroglobulinemia), monoclonal plasma cells producing the pathologic light chain can be detected in the bone marrow and blood of AL amyloid subjects (Witzig et al, 2000, McElroy et al, 1998, Perfetti et al, 1999, Kumar et al, 2005, Nowakowski et al, 2005).

Multiple myeloma is a cancer of a more aggressive plasma cell. Although this plasma cell clone generally does not generate a fibrillogenic light chain (about 95% of the time), only 50% survival is observed after 5 years, even after aggressive treatment with chemotherapeutics and stem cell rescue. Approximately 15,000 and 1,500 americans are officially diagnosed with multiple myeloma or AL amyloidosis/year. The AL amyloidosis diagnosis rate is probably underestimated by a factor of 2 or 3 since it is commonly misdiagnosed as another organ-specific diseases.

Multiple myeloma is caused by a neoplastic plasma cell which crowds out all other hematopoietic cells in the bone marrow. In contrast, AL amyloidosis is caused by an antibody (immunoglobulin) component (light chain) secreted by neoplastic plasma cells that deposits in many organs causing multi-organ failure. In AL amyloid, the plasma cells is not an aggressive, fast growing cell, and generally does not crowd out other hematopoietic cells. Death is caused by the fibrillogenic light chain.

Despite aggressive treatment approaches, multiple myeloma (MM) is a universally fatal B cell malignancy, accounting for 1-2% of all cancer deaths. See Silverberg et al. (1996) Ca. J. Clin. 46:5-27. MM is recognized clinically by the proliferation of malignant plasma cells in the bone marrow, the detection of a monoclonal protein (M protein) in the serum or urine, anemia, hypercalcemia, renal insufficiency and lytic bone lesions. See Kyle and Lust (1989) Seminars in Hematology 26:176-200. Several multiple myeloma-related plasmaproliferative disorders, such as monoclonal gammopathy of undetermined significance (MGUS) and smoldering multiple myeloma (SMM), are characterized by the detection of M protein in the serum or urine without the other clinical features of MM. MGUS, a clinically benign precursor condition of MM is more common than MM, occurring in 1% of the population over age 50 and 3% of those over age 70. Greipp and Lust (1995) Stem Cells 13:10-21. It is of great clinical importance to distinguish patients with MGUS from those with MM, as MGUS patients may be safely observed without resort to chemotherapy. The unnecessary treatment of MGUS patients with chemotherapy can lead to acute leukemia or morbidity/mortality. However, a proportion of MGUS patients develop MM, as in long-term follow-up study of 241 patients with MGUS, 59 patients (24.5%) went on to develop MM or a related disorder. See Kyle (1993) Mayo Clinic Proceedings 68:28. Thus, the prevention of multiple myeloma from MGUS would have a significant impact on the morbidity and mortality of multiple myeloma.

The current treatments for AL amyloidosis are toxic and insufficiently effective. Classically, AL amyloidosis treatment has focused on reducing the body burden of clonal plasma cells with chemotherapeutics, although shown to be effective, is not curative for multiple myeloma. This approach has had limited success in AL amyloid, and is not universally well tolerated. The median survival time with conventional chemotherapy (e.g. oral melphalan + prednisone) is 10-18 months from diagnosis (Kyle et al, 1997). In many cases where amyloid fibril deposition has compromised critical organ function, chemotherapy of any kind may be contraindicated because of high toxicity of chemotherapeutics. For the 56% of AL amyloid subjects eligible for a more aggressive regimen, for example, high dose melphalan (200 mg/m2) and autologous stem cell (ASCT) rescue, 13% die within the first 100 days and the median survival is only 4.6 years (Skinner et al, 2004). All of these subjects face a significant reduction in quality of life as they cope with transplant-related anemia, leukopenia, and other sequelae (Dispenzieri et al, 2004).

Very rarely do subjects with AL amyloidosis spontaneously achieve complete remission, due to the fact that amyloid fibrils themselves are non-immunogenic. While various therapies for amyloidosis have been investigated, such as high-dose chemotherapy, steroids, iodinated doxorubicin and stem cell replacement therapy, most therapies have limitations and/or are ineffective, in particular ineffective at curing AL amyloidosis. The majority of such therapeutic efforts in amyloidosis have focused on the development of therapies, compounds and agents that prevent protein aggregation leading to the formation of fibrils, thus removing the amyloid fibril deposits from the affected subject, as well as agents that prevent or block the initial interaction of fibrilliar proteins in tissues. For example, therapeutic efforts to reduce and remove the amyloid fibril deposits has been attempted using passively administered amyloid light chain-specific antibodies for degradation of AL amyloid deposits (O'Nuallain et al, 2006, Solomon et al, 2003). However, the use of such amyloid light chain-specific antibodies to reduce amyloid deposits has shown limited success in the clinic and have limited clinical applicability with respect to a permanent remission of the disease, because such therapeutic agents only target the amyloid deposits or their formation, which are manifestation of the disease.

### SUMMARY OF THE INVENTION

The present invention provides pharmaceutical compositions and methods for prophylactic and therapeutic treatment of disorders characterized by plasma cell disorders, malignancies and plasma cell dyscrasias.

The present invention relates to the use of immunogenic peptides for the treatment of plasma cell disorders, plasma cell dyscrasias and malignancies. The inventors have discovered a method to use immunogenic peptides to elicit an immune response towards plasma cells. Accordingly, the present invention relates to the use of immunogenic peptides of the present invention as peptide-based vaccines for the therapeutic treatment of plasma cell disorders, plasma cell dyscrasias and other malignancies.

The present invention is based on the discovery that immunogenic peptides of a region of the immunoglobulin light chain, or derivatives or fragments thereof, can bind to cells expressing MHC class I and elicit a CD8+ cytotoxic T lymphocyte (CTL) response, in particular regions of the lambda light chains or derivatives or fragments thereof. The inventors have discovered methods to elicit a peptide-specific killer T-cell activity which is capable of lysing cells that express or produce the immunoglobulins from which the immunogenic peptide is derived. Accordingly, the present invention provides a method to use immunogenic peptides to elicit an immune response towards plasma cells. In some embodiments, the present invention relates to the use of immunogenic peptides of the present invention as peptide-based vaccines for the therapeutic treatment of plasma cell disorders, plasma cell dyscrasias and other malignancies.

In some embodiments, the plasma cell disorder or plasma cell dyscrasias or other malignancy is, for example but not limited to, AL amyloidosis, multiple myeloma, AL amyloidosis-associated disorder or multiple-myeloma related hyperproliferative disorder, AL amyloid, monoclonal gammopathies, multiple myeloma, MGUS (monoclonal gammopathy of undetermined significance) and Waldenstrom's macrogloblinema.

In some embodiments, the immunogenic peptides of the present invention are a region of the immunoglobulin light chain that is produced by plasma cells, or a derivative or fragment thereof.

In some embodiments, immunogenic peptides are a region of the light chain of an immunoglobulin, or a derivative or fragment thereof, for example a region of the lambda light chain. In particular, the present invention provides immunogenic peptides from a region of an immunoglobulin that does not contain a heavy light chain. In alternative embodiments, the immunogenic peptide is from a region of the kappa light chain region of an immunoglobulin, or a fragment or derivative thereof. Any immunogenic peptide of any lambda or kappa region of the immunoglobulin, or a derivative or fragment thereof, is useful in the methods of the present invention. In some embodiments, the light chain region is a lambda 6 light chain, or lambda 1 or lambda 2 immunoglobulin light chain. In some embodiments the immunogenic peptide is lambda 6(2-10) (λ62-10) peptide which corresponds to SEQ ID NO:1 or a derivative or fragment thereof, and in other embodiments the immunogenic peptide is a heteroclitic variant, for example heteroclitic lambda 6(2-10) peptide (het λ6₂₋₁₀) which corresponds to SEQ ID NO:2 or a derivative or fragment thereof. In some embodiments, the immunogenic peptide of the present invention is a derivative or fragment of SEQ ID NO: 1 or SEQ ID NO: 2.

In one embodiment, the present invention provides a vaccine comprising an immunogenic peptide of the present invention, where the immunogenic peptide is substantially similar to a region of an immunoglobulin where the immunoglobulin is expressed by a malignant cell.

In some embodiments, the malignant cell is a plasma cell. In alternative embodiments, the malignant cell is a tumor cell. In some embodiments, the immunogenic peptide a region of the immunoglobulin light chain, for example a region of the variable region of the light chain, and in some embodiments, the region is the lambda region of the immunoglobulin light chain, or a derivative or fragment thereof. In particular, the region of the immunoglobulin used for the immunogenic peptide does not contain a heavy chain. In some embodiments, the immunogenic peptide is a region of the kappa light chain or a derivative or fragment thereof.

In some embodiments, the present invention provides methods to treat a subject with a plasma cell disorder, plasma cell dyscrasias or malignancy, the method comprising administering the subject a pharmaceutical composition comprising at least one immunogenic peptide of the present invention to a subject in need thereof.

In some embodiments, the pharmaceutical composition further comprises other antigens or helper peptides, for example but not limited to an adjuvant. Examples of such adjuvants include but without limitation, Freud's adjuvant, incomplete Freud's adjuvant, QS21, aluminum hydroxide gel, MF59 and calcium phosphate. In some embodiments, the pharmaceutical composition or vaccine comprises an immunogenic peptide and a carrier protein, and in some embodiments, the carrier protein is conjugated to the immunogenic peptide of the present invention.

In some embodiments, the subject is a mammal, and in some embodiments, the subject is human. In further embodiments, the subject has a plasma cell disorder or malignancy, or the subject is at risk of developing a plasma cell disorder or plasma cell dyscrasias.

Another aspect of the invention relates to methods to design immunogenic peptides for the treatment of plasma cell disorders and other malignancies, for example cancers and tumor malignancies. In some embodiments the invention provides methods to design immunogenic peptides substantially similar to a region of the proteins which are expressed or overexpressed by malignant cells, for example plasma cells and/or tumor cells. Accordingly, the present invention provides methods to design peptide immunogens for treatment of such disorders such as plasma cell disorders and malignancies, for example tumor malignancies. In such an embodiment, the method of the present invention comprises; (i) a screen to identify proteins expressed only in, or a higher level in the plasma cells or malignant cells from the subject afflicted with a malignancy as compared with a normal subject, (ii) assessing the immunogenic peptides that are substantially similar to a region of the identified protein for stability to form complex with MHC Class I. The method to design immunogenic peptides may further optionally comprise assessing the ability of immunogenic peptides predicated to form stable complexes with MHC Class I to elicit a CTL response in vitro or in vivo.

In some embodiments, the present invention provides methods to design immunogenic peptides to a wide range of diseases, malignancies and disorders. In some embodiments, the malignancy is cancer. In other embodiments, the disorder is an amyloidogenic disease or an amyloidogenic-associated disease, which include for example but is not limited to, amyloid-related diseases, Alzheimer's Disease, Down's syndrome, vascular dementia or cognitive impairment, type II diabetes mellitus, amyloid A (reactive), secondary amyloidosis, familial mediterranean fever, familial nephrology with urtcaria and deafness (Muckle-wells Syndrome), amyloid lambda L-chain or amyloid kappa L-chain (idiopathic, multiple myeloma or macroglobulinemia-associated) A beta 2M (chronic hemodialysis), ATTR (familial amyloid polyneuropathy (Portuguese, Japanese, Swedish), familial amyloid cardiomyopathy (Danish), isolated cardiac amyloid, (systemic senile amyloidosis) AIAPP or amylin insulinoma, atrial naturetic factor (isolated atrial amyloid), procalcitonin (medullary carcinoma of the thyroid), gelsolin (familial amyloidosis (Finnish), cyctatin C (heritiaty cerebral hemorrhage with amyloidosis (Icelandic), AApo-A-I (familial amyloidotic polyneuropathy—Iowa), AApo-A-II (accelerated senescence in mice), fibrinogen-associated amyloid; and Asor or Pr P-27 (scrapie, Creutzfeld jacob disease, Gertsmann-Straussler-Scheinker syndrome, bovine spongiform encephalitis) and subjects who are homozygous for the apolipoprotein E4 allele.

In a further embodiment, the present invention also provides method to identify peptide-specific cytotoxic T cell lympocytes (CTL) in a population of T cells.

In a further embodiment, the present invention provides methods to treat or prevent a plasma cell disorder or plasma cell dyscrasias, the method comprising administration of a pharmaceutical composition comprising an antibody directed to the immunogenic peptide of the present invention. Such a method is commonly known as passively immunization by persons or ordinary skill in the art. In such an embodiment, the subject is at risk of developing, or has a plasma cell disorder or plasma cell malignancy, for example a plasma cell dyscrasias by using the immunogenic peptides of the present invention to produce antibodies. Any means to produce the antibodies are useful in the methods of the present invention. In some embodiments, the antibodies can be, for example without limitation, polyclonal or monoclonal, recombinant, chimeric, humanized, human etc.

### BRIEF DESCRIPTION OF FIGURES

FIG. 1A-1C shows native and hererocylitic Ig λ6 framework 1-derived peptide binds to HLA-A2. The native Ig λ6₂₋₁₀ is shown in panel A, and the heteroclitic Ig λ6₂₋₁₀ is show in panel B, with the shift to the right (filled in curve) in the presence of the compared to their absence (unfilled curve) indicating Ig λ62-10 peptide binding to HLA-A2 (FL1). Panel C shows mean fluorescence as an indicator of peptide HLA-A2 binding.

FIG. 2 show Ig λ6₂₋₁₀ peptide induces a potent CTL response which lyses the λ62-10 loaded B cell lymphoblastoma (JY) cells. HHD mice were immunized with λ6 peptide lysed target cells pulsed with the λ6 peptide, but not with the negative control peptide. This killing assay confirms the ability of the native λ6 peptide immunization protocol to elicit a CTL which can kill target cells pulsed with that peptide, confirming the immunogenicity of the native λ6 peptide. Data shown is average of the 17 mice that produced a CTL response (of 24 mice immunized). **p<0.001.

FIG. 3 shows heteroclitic Ig λ6₂₋₁₀ peptide induced CTL response which recognizes heterocylitic (hc) and native lambda 6 peptides in B cell lymphoblastoma (JY) cells. HHD mice immunized with heteroclitic λ6 peptide produced T cells which lysed target cells pulsed with either heteroclitic λ6 peptide or the native λ6 peptide, but not targets pulsed with a negative control peptide. Data shown is an average of the 7 mice that produced a CTL response (of 8 mice immunized). **p<0.001, *p<0.01, †p<0.05.

FIG. 4 shows the CTL induced with Ig λ6₂₋₁₀ peptide kills JY cells transfected with full length λ6 gene and which express and process the λ6 protein and present λ6 peptides. HHD mice were immunized with λ6 peptide produced T cells which lysed targets transfected with the gene for the full length λ6 protein. This killing assay confirms that CTL from mice immunized with a peptide can kill target cells which are making the protein from which the peptide is substantially similar to a region of. Data shown is an average of 13 mice that produced a CTL response (of 16 mice immunized). **p<0.001.

FIG. 5 shows the CLT induced with Ig λ₆₋₁₀ peptide cross-reacts with Ig λ2-expressing U66 multiple myeloma plasma cells, with no response with U266 cells in the absence of peptide. HHD mice immunized with λ6 peptide produced T cells which lysed U266 target cells pulsed with the λ6 peptide as well as U266 cells not pulsed with a peptide. This killing assay confirms the ability of the CTL elicited with the λ6 peptide immunization protocol to lyse U266 cells which naturally produce a protein of the λ2 sub-type of light chain Data shown is an average of 8 mice that produced a CTL response (of 11 mice immunized). **p<0.001, †p<0.05.

FIGS. 6A-6E shows peptide/HLA-A*0201-specific T cells from mice immunized with λ6 peptide can be identified and isolated using flow cytochemistry. Panel A show a schematic of how the peptide/HLA-A*0201-specific T cells are identified by flow cytochemistry. Panels B, D and E show the isolated peptide/HLA-A*0201-specific T cells identified using flow cytochemistry, with the killing assay next to each scatter plot. Data shown are representative of two mice. Targets: ▪JY+ lambda peptide; JY+ negative peptide; ΔJY−lambda6 transfectant; □U266+ lambda peptide; ◯U266+ no peptide.

FIG. 7 shows T cells from murine Blimp₄₇₀₋₄₇₈ immunized mice kill mBlimp₄₇₀₋₄₇₈-pulsed target cells. HHD mice were immunized with native murine Blimp₄₇₀₋₄₇₈ peptide lysed target cells pulsed with the native murine Blimp₄₇₀₋₄₇₈ peptide, but not with the negative control peptide. This killing assay confirms the ability of the native Blimp₄₇₀₋₄₇₈ peptide immunization protocol to elicit a CTL which can kill target cells pulsed with that peptide, confirming the immunogenicity of the native Blimp₄₇₀₋₄₇₈ peptide. Data shown is average of the 10 mice that produced a CTL response (of 12 mice immunized). +p<0.01

FIG. 8 shows T cells from hc murine Blimp₄₇₀₋₄₇₈ immunized mice kill hc mBlimp₄₇₀₋₄₇₈-pulsed target cells. HHD mice were immunized with hc murine Blimp₄₇₀₋₄₇₈ peptide lysed target cells pulsed with the hc murine Blimp₄₇₀₋₄₇₈ peptide, but not with the negative control peptide. This killing assay confirms the ability of the heteroclitic Blimp₄₇₀₋₄₇₈ peptide immunization protocol to elicit a CTL which can kill target cells pulsed with that peptide, confirming the immunogenicity of the hc murine Blimp₄₇₀₋₄₇₈ peptide. Data shown is average of the 7 mice that produced a CTL response (of 10 mice immunized). # p<0.001, +p<0.01

FIG. 9 shows the peptide/MHC binding assay for native lambda 6 peptide and the heteroclitic lambda 6 peptide, showing the heteroclitic peptide is more stable than the native lambda 6 peptide as early as 2 hours and over a period of at least 24 hours.

### DETAILED DESCRIPTION OF THE INVENTION

### 1. General

The present invention provides pharmaceutical compositions and methods for prophylactic and therapeutic treatment of disorders characterized by plasma cell disorders, malignancies and plasma cell dyscrasias.

The present invention relates to the use of immunogenic peptides for the treatment of plasma cell disorders, plasma cell dyscrasias and malignancies. The inventors have discovered a method to use immunogenic peptides to elicit an immune response towards plasma cells. The immune response is a CD8⁺ T cell response such as a CTL or cytotoxic T lymphocyte cell response. Accordingly, the present invention relates to the use of immunogenic peptides of the present invention as peptide-based vaccines for the therapeutic treatment of plasma cell disorders, plasma cell dyscrasias and other malignancies.

In some embodiments, the plasma cell disorder or malignancy is AL amyloidosis and in some embodiments the plasma cell disorder or malignancy is multiple myeloma. In some embodiments the plasma cell disorder or malignancy is an AL amyloidosis-associated disorder.

In some embodiments, the present invention provides methods to treat and/or prevent plasma cell malignancies, for example but not limited to, plasma cell dyscrasias (such as AL amyloidosis and other plasma disorders, such as multiple myeloma) by generating an immune response thereto. The method uses an immunogenic peptide, where the immunogenic peptide is a region of the immunoglobulin light chain produced by plasma cells, for example the immunogenic peptide is an lambda light chain region of the immunogenic light chain, or a derivative or fragment thereof.

Accordingly, the present invention relates to methods to treat subjects that have cells expressing pathogenic immunoglobulins with lambda light chain subtypes 6 and/or 2, for example, plasma cell dyscrasias. In some embodiments, the present invention relates to methods to treat plasma cell malignancies, for example plasma cell dyscrasias, using compounds and immunogenic peptides generated by lambda-6 peptides, for example, treatment of plasma cell dyscrasias including but not limited to AL amyloidosis, AL amyloid, monoclonal gammopathies, multiple myeloma, MGUS (monoclonal gammopathy of undetermined significance) and Waldenstrom's macrogloblinema.

The inventors have discovered that immunogenic peptides substantially similar to a region of the immunoglobulin light chains can bind to cells expressing MHC class I and elicit a CD8⁺ cytotoxic T lymphocyte (CTL) response. Accordingly, the inventors have discovered methods to elicit a peptide-specific killer T-cell activity which is capable of lysing cells that express or produce the immunoglobulins from which the immunogenic peptide is derived.

In some embodiments, the immunogenic peptide is a region a lambda light chains of an immunoglobulin, or a derivative or fragment thereof. In alternative embodiments, the region is the kappa light chain region of an immunoglobulin. In particular, the immunogenic peptide is a region of the immunoglobulin that is not a heavy chain of the immunoglobulin. Any immunogenic peptide of a region of the any lambda or kappa immunoglobulin, or a derivative or fragment thereof is useful in the methods of the present invention. In some embodiments, the lambda light chain region is a lambda 6 light chain, or lambda 1 or lambda 2 immunoglobulin light chains, or derivatives or fragments thereof. In some embodiments the immunogenic peptide is lambda 6(2-10) (λ6₂₋₁₀) peptide or derivatives or fragments thereof, and in other embodiments the immunogenic peptide is a heteroclitic variant, for example heteroclitic lambda 6(2-10) peptide (hetλ6₂₋₁₀) or derivatives or fragments thereof.

The inventors discovered immunogenic peptides of the present invention, such as immunogenic peptides lambda 6(2-10) and het lambda 6(2-10) are capable of eliciting an immune response and capable of killing monoclonal plasma cells and human B-cells expressing lambda 6 comprising immunoglobulins, or expressing lambda 6(2-10) peptide or human B-cells that have been pulse-exposed to lambda 6(2-10) peptide. The inventors have also discovered that lambda 6(2-10) (λ6₂₋₁₀) and/or the heteroclitic lambda 6(2-10) variant peptide (het λ₆₂₋₁₀) can also target cells expressing immunoglobulins expressing lambda 2.

Another aspect of the invention relates to methods to design immunogenic peptides for the treatment of plasma cell disorders and other malignancies, for example cancers and tumor malignancies. In such an embodiment, the invention provides methods to design immunogenic peptides substantially similar to a region of the proteins which are expressed or overexpressed by malignant cells, for example plasma cells or tumor cells. Accordingly, the present invention provides methods to design peptide immunogens for treatment of such disorders such as plasma cell disorders and malignancies, for example tumor malignancies. In such an embodiment, the method of the present invention uses a screen to identify overexpressed proteins from the plasma cell or malignant cell, and then use of algorithms to assess the stability to form complexes with MHC Class I molecules. Suitable immunogenic peptides that are predicted to form stable complexes with MHC Class I molecules are then assessed for their ability to induce a CTL as discussed herein in the examples.

Another aspect of the invention relates to methods to design immunogenic peptides for the treatment of plasma cell disorders and other malignancies, for example cancers and tumor malignancies. In some embodiments the invention provides methods to design immunogenic peptides substantially similar to a region of the proteins which are expressed or overexpressed by malignant cells, for example plasma cells and/or tumor cells. Accordingly, the present invention provides methods to design peptide immunogens for treatment of such disorders such as plasma cell disorders and malignancies, for example tumor malignancies. In such an embodiment, the method of the present invention comprises; (i) a screen to identify proteins expressed only in, or a higher level in the plasma cells or malignant cells from the subject afflicted with a malignancy as compared with a normal subject, (ii) assessing the immunogenic peptides that are substantially similar to a region of the identified protein for stability to form complex with MHC Class I. The method to design immunogenic peptides may optionally comprise of assessing the ability of immunogenic peptides predicated to form stable complexes with MHC Class I to elicit a CTL response in vitro or in vivo using the methods of the present invention.

In some embodiments, the present invention also provides methods to design immunogenic peptides to a wide range of diseases, malignancies and disorders. In some embodiments, the malignancy is cancer. In other embodiments, the disorder is an amyloidogenic disease or an amyloidogenic-associated disease. Amyloidogenic-associated disease include, for example without limitation, amyloid-related diseases, Alzheimer's Disease, Down's syndrome, vascular dementia or cognitive impairment, type II diabetes mellitus, amyloid A (reactive), secondary amyloidosis, familial mediterranean fever, familial nephrology with urtcaria and deafness (Muckle-wells Syndrome), amyloid lambda L-chain or amyloid kappa L-chain (idiopathic, multiple myeloma or macroglobulinemia-associated) A beta 2M (chronic hemodialysis), ATTR (familial amyloid polyneuropathy (Portuguese, Japanese, Swedish), familial amyloid cardiomyopathy (Danish), isolated cardiac amyloid, (systemic senile amyloidosis) AIAPP or amylin insulinoma, atrial naturetic factor (isolated atrial amyloid), procalcitonin (medullary carcinoma of the thyroid), gelsolin (familial amyloidosis (Finnish), cyctatin C (heritiaty cerebral hemorrhage with amyloidosis (Icelandic), AApo-A-I (familial amyloidotic polyneuropathy—Iowa), AApo-A-II (accelerated senescence in mice), fibrinogen-associated amyloid; and Asor or Pr P-27 (scrapie, Creutzfeld jacob disease, Gertsmann-Straussler-Scheinker syndrome, bovine spongiform encephalitis) and person who are homozygous for the apolipoprotein E4 allele. Accordingly, the present invention provides methods to design peptide immunogens to peptides or proteins that form deposits in amyloidogenic diseases.

In a further embodiment, the present invention also provides method to identify peptide-specific cytotoxic T cell lympocytes (CTL) in a population of T cells. In such an embodiment, the method involves tagging the peptide immunogen with a detectable marker, for example a fluorescence marker which can be used it identify cells which bind the peptide immunogen of the present invention, for example by using FACs or other cell sorting methods.

In a further embodiment, the present invention provides methods to treat or prevent a plasma cell disorder or plasma cell dyscrasias, the method comprising administration of a pharmaceutical composition comprising an antibody directed to the immunogenic peptide of the present invention. Such a method is commonly known as passive immunization by persons of ordinary skill in the art. In such an embodiment, the subject is at risk of developing, or has a plasma cell disorder or plasma cell malignancy, for example a plasma cell dyscrasias by using the immunogenic peptides of the present invention to produce antibodies. In alternative embodiments, any peptide immunogen of the present invention can be used for antibody production, for example peptide immunogen substantially similar to a protein expressed by a tumor cell and/or a peptide that forms deposits in amyloidogenic diseases or amyloidogenic-associated disorders. Any means to produce the antibodies are useful in the methods of the present invention. In some embodiments, the antibodies can be polyclonal or monoclonal, recombinant, chimeric, humanized, human etc.

### II. Definitions

Unless otherwise indicated, all terms used herein have the same meaning as they would to one skilled in the art of the present invention. Practitioners are particularly directed to Sambrook, et al. (1989) Molecular Cloning: A Laboratory Manual (Second Edition), Cold Spring Harbor Press, Plainview, N.Y. and Ausubel, F. M., et al. (1998) Current Protocols in Molecular Biology, John Wiley Sons, New York, N.Y., for definitions, terms of art and standard methods known in the art of biochemistry and molecular biology. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. Unless otherwise indicated, nucleic acids are written left to right in 5′ to 3′ orientation; amino acid sequences are written left to right in amino to carboxy orientation, respectively. The headings provided herein are not limitations of the various aspects or embodiments of the invention which can be had by reference to the specification as a whole. Accordingly, the terms defined immediately below are more fully defined by reference to the specification as a whole.

It is understood that this invention is not limited to the particular methodology, protocols, and reagents described, as these may be varied to produce the same result.

The term “amyloidosis” as used herein refers to a condition in which abnormal protein deposits in various tissues. These protein deposits damage the tissues and interfere with the function of the involved organ. The abnormal protein deposits are called amyloid, hence the name of this group of diseases. Amyloidosis occurs in multiple forms: spontaneous, hereditary, and also in some instances is a result from a cancer of the blood cells called myeloma. Hereditary amyloidosis is an inherited form, and in some occasions is transmitted as an autosomal dominant trait.

The term “AL amyloidosis” as used herein refers to the disease or disorder from AL amyloid deposits, or the formation of amyloid deposits comprising monoclonal immunoglobulin light chain.

The term “Multiple myeloma” or “multiple myeloma” or “MM” are used interchangeably herein is a disorder characterized by the proliferation of malignant plasma cells in the bone marrow. Multiple myeloma is a type of cancer of the plasma cells in bone marrow. Also encompassed in the term multiple myeloma are multiple myeloma-related plasma proliferative disorders, such as, for example but not limited to monoclonal gammopathy of undetermined significance (MGUS) and smoldering multiple myeloma (SMM). Multiple myeloma is sometimes referred to by other names, for example but not limited to, plasma cell dyscrasia, plasma cell myeloma, malignant plasmacytoma; plasmacytoma of bone.

The term “dyscrasias” as used herein is a nonspecific term that refers to any disease or disorder, although it usually refers to blood diseases. The term “plasma cell dyscrasias” as used herein refers to disorders of the plasma cells.

The term ‘disorder’ or ‘disease’ used interchangeably herein, refers to any alteration in the state of the body or of some of its organs, interrupting or disturbing the performance of the functions and/or causing symptoms such as discomfort, dysfunction, distress, or even death to the person afflicted or those in contact with the person. A disease or disorder can also relate to distemper, ailing, ailment, malady, disorder, sickness, illness, complaint, indisposition, affection.

The term ‘malignancy’ and ‘cancer’ are used interchangeably herein, refers to diseases that are characterized by uncontrolled, abnormal growth of cells and also refers to any disease of an organ or tissue in mammals characterized by poorly controlled or uncontrolled multiplication of normal or abnormal cells in that tissue and its effect on the body as a whole. Cancer cells can spread locally or through the bloodstream and lymphatic system to other parts of the body. Cancer diseases within the scope of the definition comprise benign neoplasms, dysplasias, hyperplasias as well as neoplasms showing metastatic growth or any other transformations like e.g. leukoplakias which often precede a breakout of cancer. A ‘malignant cell’ as used herein is intended to refer to the cancer causing cell, or a cell that has uncontrolled proliferation. The term “cancer”, as used herein refers to a cellular proliferative disease in a human or animal subject.

The term “tumor” or “tumor cell” used interchangeably herein refers to the tissue mass or tissue type or cell type that is undergoing uncontrolled proliferation.

The term ‘toxin’ as referred to herein is intended to encompass any entity, typically but not exclusivley a polypeptide which is capable of being cytotoxic, i.e. that is toxic to a cell. The term “cytotoxin” as used herein refers to a toxic entity that is specifically toxic to cells. The term “immunotoxin” as used herein refers to a polypeptide comprising a toxic entity that is specifically designed to target specific cells by use of a targeting region. In the present invention, an immunotoxin is a antibody directed to the peptide immunogen of the present invention conjugated to a toxin or toxin molecule.

The term “derivative” as used herein refers to peptides which have been chemically modified by techniques such as ubiquitination, labeling, pegylation (derivatization with polyethylene glycol), and insertion, deletion or substitution of amino acids, including insertion, deletion and substitution of amino acids and other molecules (such as amino acid mimetics or unnatural amino acids) that do not normally occur in the peptide sequence that is basis of the derivative, for example but not limited to insertion of ornithine which do not normally occur in human proteins. The term “derivative” is also intended to encompass all modified variants of the immunogenic peptide, for example but not limited to for example homologue regions of the regions of the immunglobulin, variants, functional derivatives, analogues and fragments thereof, as well as peptides with substantial identity as compared to the reference peptide to which they refer to. The term derivative is also intended to encompass aptamers, peptidomimetics and retro-inverso peptides of the reference peptide to which the refer to.

As used herein, the terms “homologous” or “homologues” are used interchangeably, and when used to describe a polynucleotide or polypeptide, indicates that two polynucleotides or polypeptides, or designated sequences thereof, when optimally aligned and compared, for example using BLAST, version 2.2.14 with default parameters for an alignment (see herein) are identical, with appropriate nucleotide insertions or deletions or amino-acid insertions or deletions, in at least 70% of the nucleotides or amino acid residues, usually from about 75% to 99%, and more preferably at least about 98 to 99% of the nucleotides or amino acid residues. The term “homolog” or “homologous” as used herein also refers to homology with respect to structure and/or function. With respect to sequence homology, sequences are homologs if they are at least 50%, at least 60 at least 70%, at least 80%, at least 90%, at least 95% identical, at least 97% identical, or at least 99% identical. Determination of homologs of the genes or peptides of the present invention can be easily ascertained by the skilled artisan. Homologous sequences can be the same functional gene in different species.

The term “analog” as used herein of a molecule, such as a peptide immunogens as disclosed herein, for example SEQ ID NOs: 1 to 8 refers to a molecule similar in function to either the entire molecule of a fragment thereof. The term “analogue” is indented to include allelic, species and variants. Analogs typically differ from naturally occurring peptides at one or a few positions, often by virtue of conservative substitutions. Analogs typically exhibit at least 80 or 90% sequence identity with the natural peptides or the peptide sequence they are an analogue of. In some embodiments, analogs also include unnatural amino acids or modifications of N or C terminal amino acids. Examples of unnatural amino acids are acedisubstituted amino acids, N-alkyl amino acids, lactic acid, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, δ-N-methylarginine. Fragments and analogs can be screened for prophylactic or therapeutic efficacy in transgenic animal models as described below.

The term “substantial identity” as used herein refers to two peptide sequences, when optimally aligned, such as by the programs GAP or BESTFIT using default gap weights, share at least about 65%, at least about . . . 70%, at least about . . . 80%, at least about . . . 90% sequence identity, at least about . . . 95% sequence identity or more (e.g., 99% sequence identity or higher). In some embodiments, residue positions which are not identical differ by conservative amino acid substitutions.

The term “antibody” is used to include intact antibodies and binding fragments thereof. Typically, fragments compete with the intact antibody from which they were derived for specific binding to an antigen. Optionally, antibodies or binding fragments thereof, can be chemically conjugated to, or expressed as, fusion proteins with other proteins.

The term “anti-idiotype antibody” or “anti-Id” or “α-Id” are used interchangeably herein, refers to an antibody directed against the antigen specific part of the sequence of an antibody or immunoglobulin or T-cell receptor and thus recognize the binding sites of other antibodies. Without being bound by theory, an anti-idiotype antibody can inhibit a specific immune response and they are important to the regulation of the immune system. As used herein, anti-idiotype antibody is an antibody directed against the lambda light chain of an antibody, and in some embodiment antibodies directed against immunoglobulin light chains comprising lambda 6 light chains.

The term “epitope” or “antigenic determinant” refers to a site on an antigen to which B and/or T cells respond. The term can also refer to the part of an antigen that binds to the antigen-binding region of an antibody. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., J Inf. Dis. 170, 1110-19 (1994)), by antigen-dependent killing (cytotoxic T lymphocyte assay, Tigges et al., J. Immunol. 156, 3901-3910) or by cytokine secretion.

The term “immunological” or “immune” as used herein with respect to an immunological or immune response, refers to the development of a humoral (antibody mediated) and/or a cellular (mediated by antigen-specific T cells or their secretion products) response directed against an amyloid peptide in a recipient subject. Such a response can be an active response induced by administration of an immunogen or immunogenic peptide to a subject or a passive response induced by administration of antibody or primed T-cells that are directed towards the immunogen. A cellular immune response is elicited by the presentation of polypeptide epitopes in association with Class I or Class II MHC molecules to activate antigen-specific CD4⁺ T helper cells and/or CD8⁺ cytotoxic T cells. Such a response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays (see Burke, supra; Tigges, supra). The relative contributions of humoral and cellular responses to the protective or therapeutic effect of an immunogen can be distinguished by separately isolating IgG and T-cells from an immunized syngeneic animal and measuring protective or therapeutic effect in a second subject, or a cytotoxic T-cell response (CTL) as disclosed in the Examples.

An “immunogenic agent” or “immunogen” as used herein is capable of inducing an immunological response against itself on administration to a subject, optionally in conjunction with an adjuvant.

An “immunogenic agent” or “immunogen” or “antigen,” also referred to as “immunogenic peptide” is a molecule that is capable of inducing an immunological response against itself upon administration to a patient, either in conjunction with, or in the absence of, an adjuvant.

The term “adjuvant” refers to a compound that when administered in conjunction with an immunogen augments the immune response to the antigen, but when administered alone does not generate an immune response to the antigen. Adjuvants can augment an immune response by several mechanisms including lymphocyte recruitment, stimulation of B and/or T cells, and stimulation of macrophages.

The term “amyloid disease” or “amyloidosis” refers to any of a number of disorders which have as a symptom or as part of its pathology the accumulation or formation of plaques or amyloid plaques. An “amyloid plaque” is an extracellular deposit composed mainly of proteinaceous fibrils. Generally, the fibrils are composed of a dominant protein or peptide; however, the plaque may also include additional components that are peptide or non-peptide molecules, as described herein.

An “amyloid component” is any molecular entity that is present in an amyloid plaque including antigenic portions of such molecules. Amyloid components include but are not limited to proteins, peptides, proteoglycans, and carbohydrates. A “specific amyloid component” refers to a molecular entity that is found primarily or exclusively in the amyloid plaque of interest.

An “agent” is a chemical molecule of synthetic or biological origin. In the context of the present invention, an agent is generally a molecule that can be used in a pharmaceutical composition.

The terms “polynucleotide” and “nucleic acid,” as used interchangeably herein refer to a polymeric molecule having a backbone that supports bases capable of hydrogen bonding to typical polynucleotides, where the polymer backbone presents the bases in a manner to permit such hydrogen bonding in a sequence specific fashion between the polymeric molecule and a typical polynucleotide (e.g., single-stranded DNA). Such bases are typically inosine, adenosine, guanosine, cytosine, uracil and thymidine. Polymeric molecules include double and single stranded RNA and DNA, and backbone modifications thereof, for example, methylphosphonate linkages.

The term “mutant” refers to any change in the genetic material of an organism, in particular a change (i.e., deletion, substitution, addition, or alteration) in a wild-type polynucleotide sequence or any change in a wild-type protein sequence. The term “variant” is used interchangeably with “mutant”. Although it is often assumed that a change in the genetic material results in a change of the function of the protein, the terms “mutant” and “variant” refer to a change in the sequence of a wild-type protein regardless of whether that change alters the function of the protein (e.g., increases, decreases, imparts a new function), or whether that change has no effect on the function of the protein (e.g., the mutation or variation is silent). The term mutation is used interchangeably herein with polymorphism in this application.

The term “polypeptide” as used herein refers to a compound made up of a single chain of amino acid residues linked by peptide bonds. The term “protein” may be synonymous with the term “polypeptide” or may refer to a complex of two or more polypeptides. The terms “polypeptide,” “peptide” and “protein” are used herein to refer to a polymer of amino acid residues, and are not limited to a minimum length. Peptides, oligopeptides, dimers, multimers, and the like, are also composed of linearly arranged amino acids linked by peptide bonds, and whether produced biologically, recombinantly, or synthetically and whether composed of naturally occurring or non-naturally occurring amino acids, are included within this definition. Both full-length proteins and fragments thereof are encompassed by the definition. The terms also include co-translational (e.g., signal peptide cleavage) and post-translational modifications of the polypeptide, such as, for example, disulfide-bond formation, glycosylation, acetylation, phosphorylation, proteolytic cleavage (e.g., cleavage by furins or metalloproteases), and the like. Furthermore, for purposes of the present invention, a “polypeptide” refers to a protein that includes modifications, such as deletions, additions, and substitutions (generally conservative in nature as would be known to a person in the art), to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods. Polypeptides or proteins are composed of linearly arranged amino acids linked by peptide bonds, but in contrast to peptides, has a well-defined conformation. Proteins, as opposed to peptides, generally consist of chains of 100 or more amino acids. The terms apply to amino acid polymers in which one or more amino acid residue is an artificial chemical analog of a corresponding naturally occurring amino acid, as well as to naturally occurring amino acid polymers.

The term “peptide” also refers to a compound composed of amino acid residues linked by peptide bonds. For the purposes of the present invention, the term “peptide” as used herein typically refers to a sequence of amino acids of made up of a single chain of D- or L-amino acids or a mixture of D- and L-amino acids joined by peptide bonds. Generally, peptides contain at least two amino acid residues and are less than about 100 amino acids, more preferably less than about 50 amino acids in length. As used herein, the term “protein fragment” may also be read to mean a peptide.

The incorporation of non-natural amino acids, including synthetic non-native amino acids, substituted amino acids, or one or more D-amino acids into the peptides (or other components of the composition, with exception for protease recognition sequences) is desirable in certain situations. D-amino acid-containing peptides exhibit increased stability in vitro or in vivo compared to L-amino acid-containing forms. Thus, the construction of peptides incorporating D-amino acids can be particularly useful when greater in vivo or intracellular stability is desired or required. More specifically, D-peptides are resistant to endogenous peptidases and proteases, thereby providing better oral trans-epithelial and transdermal delivery of linked drugs and conjugates, improved bioavailability of membrane-permanent complexes (see below for further discussion), and prolonged intravascular and interstitial lifetimes when such properties are desirable. The use of D-isomer peptides can also enhance transdermal and oral trans-epithelial delivery of linked drugs and other cargo molecules. Additionally, D-peptides cannot be processed efficiently for major histocompatibility complex class II-restricted presentation to T helper cells, and are therefore less likely to induce humoral immune responses in the whole organism. Peptide conjugates can therefore be constructed using, for example, D-isomer forms of cell penetrating peptide sequences, L-isomer forms of cleavage sites, and D-isomer forms of therapeutic peptides. In some embodiments, the peptide immunogens, such as SEQ ID NO:1-8 can be comprised of D- or L-amino acid residues, as use of naturally occurring L-amino acid residues has the advantage that any break-down products should be relatively non-toxic to the cell or organism.

A “fibril peptide” or “fibril protein” refers to a monomeric or aggregated form of a protein or peptide that forms fibrils present in plaques.

A “pharmaceutical composition” refers to a chemical or biological composition suitable for administration to a mammalian individual. Such compositions may be specifically formulated for administration via one or more of a number of routes, including but not limited to, oral, parenteral, intravenous, intraarterial, subcutaneous, intranasal, sublingual, intraspinal, intracerebroventricular, and the like.

A “pharmaceutical excipient” or a “pharmaceutically acceptable excipient” is a carrier, usually a liquid, in which an active therapeutic agent is formulated. The excipient generally does not provide any pharmacological activity to the formulation, though it may provide chemical and/or biological stability, release characteristics, and the like. Exemplary formulations can be found, for example, in Remington's Pharmaceutical Sciences, 19^{th} Ed., Grennaro, A., Ed., 1995.

A “glycoprotein” as use herein is protein to which at least one carbohydrate chain (oligopolysaccharide) is covalently attached. A “proteoglycan” as used herein is a glycoprotein where at least one of the carbohydrate chains is a glycosaminoglycan, which is a long linear polymer of repeating disaccharides in which one member of the pair usually is a sugar acid (uronic acid) and the other is an amino sugar.

The term “subject” includes human and other mammalian subjects that receive either prophylactic or therapeutic treatment. The term “subject” and “individual” are used interchangeably herein, and refer to an animal, for example a human, to whom treatment, including prophylactic treatment, with the cells according to the present invention, is provided. For treatment of those infections, conditions or disease states which are specific for a specific animal such as a human patient, the term patient refers to that specific animal. The term “donor” is used to describe an individual (animal, including a human) who or which donates umbilical cord blood or umbilical cord blood cells for use in a patient. The “non-human animals” of the invention include mammals such as rats, mice, rabbits, sheep, cats, dogs, cows, pigs, and non-human primates.

The term “treating” as used herein is intended to encompass curing as well as ameliorating at least one symptom of the condition or disease. For example, in the case of AL amyloidosis, treatment includes a reduction in immunoglobulin-producing plasma cells. Evidence of treatment may be clinical or sub-clinical. The terme “treatment and/prophylaxis” refers generally to afflicting a subject, tissue or cell to obtain a desired pharmacologic and/or physiologic effect. The effect may be prophylactic in terms of completely or partially preventing a disease or sign or symptom thereof, and/or may be therapeutic in terms of a partial or complete cure of a disease.

The term “tissue” is intended to include intact cells, blood, blood preparations such as plasma and serum, bones, joints, muscles, smooth muscles, and organs.

The term “linker peptide” includes reference to a peptide within an antibody binding fragment (e.g., Fv fragment) which serves to indirectly bond the variable heavy chain to the variable light chain.

The term “naked polynucleotide” refers to a polynucleotide not complexed with colloidal materials. Naked polynucleotides are sometimes cloned in a plasmid vector.

The terms “significantly different than,” “statistically significant,” “significantly higher (or lower) than,” and similar phrases refer to comparisons between data or other measurements, wherein the differences between two compared individuals or groups are evidently or reasonably different to the trained observer, or statistically significant (if the phrase includes the term “statistically” or if there is some indication of statistical test, such as a p-value, or if the data, when analyzed, produce a statistical difference by standard statistical tests known in the art).

The term “effective amount” as used herein refers to the amount of therapeutic agent of pharmaceutical composition to alleviate at least some of the symptoms of the disease or disorder.

The term “encode” as it is applied to polynucleotides refers to a polynucleotide which is said to “encode” a polypeptide or protein if, in its native state or when manipulated by methods well known to those skilled in the art, it can be transcribed to produce the RNA which can be translated into an amino acid sequence to generate the polypeptide and/or a fragment thereof. The antisense strand is the complement of such a nucleic acid, and the encoding sequence can be deduced therefrom.

The term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

The term “viral vectors” refers to the use as viruses, or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g EPV and EBV vectors.

The term “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically,” “peripheral administration” and “administered peripherally” as used herein mean the administration of a compound, drug or other material other than directly into the central nervous system, such that it enters the animal's system and, thus, is subject to metabolism and other like processes, for example, subcutaneous administration.

The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

The terms “lower”, “reduced”, “reduction” or “decrease” or “inhibit” are all used herein generally to mean a decrease by a statistically significant amount. However, for avoidance of doubt, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. When “decrease” or “inhibition” is used in the context of the level of expression or activity of a gene, it refers to a reduction in protein or nucleic acid level or activity in a cell, a cell extract, or a cell supernatant. For example, such a decrease may be due to reduced RNA stability, transcription, or translation, increased protein degradation, or RNA interference. Preferably, this decrease is at least about 5%, at least about 10%, at least about 25%, at least about 50%, at least about 75%, at least about 80%, or even at least about 90% of the level of expression or activity under control conditions.

The terms “increased”, “increase” or “enhance” or “higher” are all used herein to generally mean an increase by a statically significant amount; for the avoidance of any doubt, the terms “increased”, “increase” or “enhance” or “higher” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. When “increase” is used in the context of the expression or activity of a gene or protein, it refers to a positive change in protein or nucleic acid level or activity in a cell, a cell extract, or a cell supernatant. For example, such a increase may be due to increased RNA stability, transcription, or translation, or decreased protein degradation. Preferably, this increase is at least 5%, at least about 10%, at least about 25%, at least about 50%, at least about 75%, at least about 80%, at least about 100%, at least about 200%, or even about 500% or more over the level of expression or activity under control conditions.

Compositions or methods “comprising” one or more recited elements may include other elements not specifically recited. For example, a composition that comprises a fibril component peptide encompasses both the isolated peptide and the peptide as a component of a larger polypeptide sequence. By way of further example, a composition that comprises elements A and B also encompasses a composition consisting of A, B and C.

The articles “a” and “an” are used herein to refer to one or to more than one (i.e., to at least one) of the grammatical object of the article. By way of example, “an element” means one element or more than one element.

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

The terms “comprising” means “including principally, but not necessary solely”. Furthermore, variation of the word “comprising”, such as “comprise” and “comprises”, have correspondingly varied meanings. The term “consisting essentially” means “including principally, but not necessary solely at least one”, and as such, is intended to mean a “selection of one or more, and in any combination.”

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Other features and advantages of the invention will be apparent from the following Detailed Description, the drawings, and the claims.

### III. Peptide Immunogens of Immunoglobulin Light Chains

In some embodiments of the present invention relates to immunogenic peptides, also referred to herein as “peptide immunogens”, of a region of the immunoglobulin light chain. In some embodiments, the immunogenic peptide is a region of the variable region of the immunoglobulin light chain, or a fragment or derivative thereof, and in some embodiments, the immunogenic peptide is a region of the lambda region or kappa region, or a derivative or fragment thereof, of the immunoglobulin light chain In some embodiments, the immunoglobulin is produced by plasma cells, and in alternative embodiments the immunoglobulin is produced by any cell of the lymphoid series, for example but not limited to, lympocytes

In some embodiments the immunoglobulin is a neoplasm immunoglobulin or neoplasm antibody, for example an immunoglobulin induced by antigens specific for tumors other than the normally occurring histocompatibility antigens.

An immunoglobulin is a term commonly known in the art to describe a specific protein substance that is produced by plasma cells to aid in fighting infection. Immunoglobulins or immunoglobulin molecule is used interchangeably in the art to refer to an antibody. Immunoglobulins or antibodies have a specific amino acid sequence by virtue of which it interacts only with the antigen that induced its synthesis in cells of the lymphoid series, for example immunoglobulin-producing plasma cells, or with antigen closely related to it.

Antibodies are soluble glycoproteins of the immunoglobulin superfamily. When attached to the surface of the B cell, the membrane-bound form of the immunoglobulin is sometimes referred to as the B cell receptor (BCR), and thus peptide immunogens substantially similar to a region of the B cell receptors are also encompassed for use in the methods of the present invention.

Without being bound by theory, immunoglobulins have a basic antibody structural unit, which is known to comprise a tetramer of subunits. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 more amino acids. (See generally, Fundamental Immunology (Paul, W., ed., 2nd ed. Raven Press, N.Y., 1989), Ch. 7 (incorporated by reference in its entirety for all purposes).

The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md., 1987 and 1991), or Chothia Lesk, J. Mol. Biol. 196:901-917 (1987); Chothia et al., Nature 342:878-883 (1989).

The lambda region of the light chain of the immunoglobulins has a molecular weight of approximately 22 kD and constitutes about 40% of all light chains. Immunoglobulins with lambda light chains are well known by persons in the art and can be recognized serologically as well as by their specific amino acid sequence.

The kappa region of the light chain of the immunoglobulins has a molecular weight of approximately 22 kD, and constitutes about 60% of all light chains. Immunglobulins with kappa light chains can be recognized serologically as well as by their specific amino acid sequence.

### IV. Immunogenic Peptides for Plasma Cell Disorders and Plasma Cell Dyscrasias

In one embodiment, the immunogenic peptide of the present invention is a region of the lambda light chain or a derivative or fragment thereof. In some embodiments, the immunogenic peptide useful in the present invention is a naturally occurring form of a region of the lambda light chain, herein also termed “naïve”, and in some embodiments, it is the naturally occurring human form of lambda light chain. In alternative embodiments, the immunogenic peptide of the present invention is an active fragment, analogue or variant of a region of a lambda light chain, for example but not limited to a heteroclitic peptide or a peptide with a variation at least one amino acid relative to the native peptide sequence. In some embodiments, the variations can be a substitution, for example but not limited to a conservative or non-conservative substitution, deletion or insertion of an amino acid. In some embodiments, the variation can be a modification with a synthetic entity, such as a synthetic amino acid or the like.

In some embodiments, the immunogenic peptides are from the lambda light chain, or a derivative or fragment thereof. In some embodiments the immunogenic peptides are from the lambda 6 light chain, or a derivative or fragment thereof. In alternative embodiments, the immunogenic peptides of the present invention are from the lambda 1, 2, 3, 4, 5, 6, 7, 8, 9 and 10 lambda light chains, or are derivatives or fragments thereof. In some embodiments, the immunogenic peptides are from the immunoglobulin light chain that does not contain a heavy chain.

Examples of immunogenic peptides of the present invention are, but not limited to, lambda 6(2-10): MLTQPHSV (SEQ ID NO:1); heteroclitic lambda 6 (2-10) YMLTQPHSV (SEQ ID NO:2); heteroclitic lambda 6 (2-10) FLLTQPHSV (SEQ ID NO:3); lambda 1 (2-10) SVLTQPPSV (SEQ ID NO: 4); lambda 2 (2-10) SELTQPASV (SEQ ID NO:5); or lambda 3 (2-10) SELTQPPSV (SEQ ID NO: 6). In some embodiments, the immunogenic peptide is a derivative or fragment of SEQ ID NOS 1 to 6.

In some embodiments, the immunogenic peptide is a region of a protein expressed from a plasma cell, or a derivative or fragment thereof. In some embodiments, such a protein expressed by the plasma cell is, for example but not limited to Blimp-1, which is required for plasma cell maintenance and antibody production. Accordingly, in some embodiments the immunogenic peptide is a region of the Blimp1 protein, and in some embodiments, an immunogenic peptide of the present invention is Blimp₄₇₀₋₄₇₈ SLFRLYPV (SEQ ID NO:7) or heteroclitic Blimp₄₇₀₋₄₇₈ YLFRLYPV (SEQ ID NO:8), or are derivative or fragments thereof.

In alternative embodiments, the immunogenic peptide of the present invention is any peptide of a region of a protein expressed by plasma cells that has specific binding to MHC Class I, as demonstrated in the Examples. Binding affinities and prediction of the stability to complex with MHC Class I molecules can be predicted by algorithms that are known by persons of ordinary skill in the art, for example but not limited to theses used in the Examples, such as NIH-BIMAS algorithm, SYFPEITHI algorithm and Boston University ZLAB algorithm as well as others that are available on the internet or web. In some embodiments, a predicted binding or stability between the peptide immunogen of the present invention and the MHC Class I molecules using such algorithms means they will have binding in vivo. In some embodiments, the binding affinities will be at least 10³, 10⁴ and 10⁵, 10⁶, 10⁷, 10⁸, 10⁹ M⁻¹, or 10¹⁰ M⁻¹. Affinities greater than 10⁸ M⁻¹ are also useful in the methods of the present invention.

It will be understood by those skilled in the art and aware of this invention that the standard lambda construct is identical to the standard kappa construct, with respect to the interface framework but for a lysine residue at position 38 and a phenylalanine residue at position 36. Thus, the immunogenic peptides of the present invention can be selected from immunoglobulins consisting of kappa or lambda light chains. In some embodiments, the immunogenic peptides are a region of the kappa region of the immunoglobulin light chain, or a derivative or fragment thereof. In some embodiments, the immunogenic peptides are a region of kappa 1, or kappa 2, or kappa 3 or kappa 4 immunoglobulin light chain, or a derivative or fragment thereof.

In some embodiments, the immunogenic peptides of the present invention trigger the production of anti-idiotypic (anti-Id) antibodies against immunoglobulins and monoclonal antibodies, for example immunoglobulins produced by plasma cells and B cells. Use of such anti-idiotypic antibodies is also encompassed for use in the present invention. Such anti-Id antibodies mimic the antigen, for example they mimic a region of the lambda 6 light chain and generate an immune response to it (see Essential Immunology (Roit ed., Blackwell Scientific Publications, Palo Alto, 6th ed., p. 181).

In further embodiments, analogues or derivatives of the immunogenic peptides of the present invention are useful in the methods and pharmaceutical compositions as discussed herein. Analogs of immunogenic peptides as disclosed herein, includes for example but is not limited to all allelic, species and induced variants. Analogs typically differ from naturally occurring peptides at one or a few positions, often by virtue of conservative substitutions. Analogs typically exhibit at least 80 or 90% sequence identity with natural peptides. Some analogs also include unnatural amino acids or modifications of N or C terminal amino acids. Examples of unnatural amino acids are acedisubstituted amino acids, N-alkyl amino acids, lactic acid, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, δ-N-methylarginine. Fragments and analogs can be screened for binding to MHC Class II complex and their prophylactic or therapeutic efficacy in the methods as described in the Examples herein.

In some embodiments of the invention, the immunogenic peptides as disclosed herein can have a sequence of at least 3, 4, 5, 6, 7, 8, 9, 10, 12, 14, 16 or 20 consecutive amino acids from the natural peptide. In some embodiments, the peptide is a 9 mer. In some embodiments, the immunogenic peptide is between 3-9 amino acids, and in alternative embodiments the immunogenic peptides of the present invention is between 9-20 consecutive amino acids. In some embodiments, the immunogenic peptide if between 3-20 amino acids, or between 5-15 amino acids or between 5-20 amino acids. In some embodiments, the immunogenic peptide of the present invention is greater than 20 amino acids.

In some embodiments, a peptide immunogen as disclosed herein, for example SEQ ID NOs: 1 to 8 can comprise essentially the native or wild type peptide of SEQ ID NOs 1-8, and additional amino acids attached to one or both of the C- or N-terminal of the peptide. By way of example only, a peptide immunogen useful in the methods and compositions as disclosed herein can comprise SEQ ID NO:1 and then at least 1, at least 2, at least 3, at least 4, at least 5, at least 6, at least 7, at least 8, at least about 9, at least about 10, at least about . . . 15, or more than 15 amino acids at the C- and/or the N-terminal of SEQ ID NO: 1. Any number of amino acids can be added to one or both ends of a peptide immunogen as disclosed herein, so long as the additional amino acid resides do not impair the peptide immunogen to bind to its desired target. Such peptide immunogens with additional amino acids can be assessed as compared to the same peptide immunogen without the additional amino acid residues using the assays as disclosed herein, such as induction of a peptide-specific CTL response in T cells as disclose in Example 3 and induction of a peptide-mediated CTL response in vivo in Example 3. It should be noted that if additional amino acid residues are added to both the C- and N-terminal of a peptide immunogen as disclosed herein, such as SEQ ID NOs: 1-8, then number of residues added to either end can be different, for example 3 amino acids can be added to the N-terminal and 1 amino acid can added to the C-terminal, or vice versa.

In another embodiment, a peptide immunogen as disclosed herein, for example SEQ ID NOs: 1 to 8 can comprise a fragment, derivative or variant of the native or wild type peptide of SEQ ID NOs 1-8, and additional amino acids attached to one or both of the C- or N-terminal of the peptide. By way of example only, a peptide immunogen useful in the methods and compositions as disclosed herein can comprise a fragment of SEQ ID NO:1, such as least 3, 4, 5, 6, 7 or 8 consecutive amino acids from the SEQ ID NO: 1 and additionally at least 1, at least 2, at least 3, at least 4, at least 5, at least 6, at least 7, at least 8, at least about 9, at least about 10, at least about . . . 15, or more than 15 amino acids at the C— and/or the N-terminal of SEQ ID NO:1.

As another non-limiting example, a peptide immunogen useful in the methods and compositions as disclosed herein can comprise a variant of SEQ ID NO:1, such as a variant with at least 1, 2, 3, 4 or 5 or more amino acids variations, such as deletions, substitutions, insertions or any combination thereof in SEQ ID NO: 1 and additionally at least 1, at least 2, at least 3, at least 4, at least 5, at least 6, at least 7, at least 8, at least about 9, at least about 10, at least about . . . 15, or more than 15 amino acids at the C- and/or the N-terminal of SEQ ID NO:1.

Generally, persons skilled in the art will appreciate that the immunogenic peptides designed in accordance with this aspect of the invention can be screened for cross-reactivity with the MHC Class I complex as described by the methods herein, as well as their cross reactivity for generating a CTL immune response towards other peptides expressed from plasma cells, for example immunoglobulin with lambda 1, 2, 3, or 6 light chains, or immunoglobulins with kappa light chains and/or prophylactic or therapeutic efficacy in transgenic animal models for plasma cell malignancies or AL amyloidosis or multiple myeloma as described herein. Such immunogenic peptides of the present invention may be used in pharmaceutical compositions of the present invention for the treatment of plasma cell malignancies.

A substantial number of sequences of amyloidogenic immunoglobulin in light chains have been obtained either by direct amino acid sequencing of protein isolated from patient urine or from amyloid deposits or by nucleotide sequencing of cDNAs cloned from plasma cells of patients with AL-type amyloidosis, which are useful in the methods of the present invention but no particular common sequences have been identified as obviously correlating with the pathogenic properties of the amyloid-associated light chains. See Natvig et al., supra; Aucouturier et al., which is incorporated herein in its entirety by reference, and the references cited therein are incorporated herein by reference in their entirety.

It is contemplated that modified version of the immunogenic peptides of the present invention can be used, for example, peptides with amino acids substitutions. In some embodiments, the immunogenic peptides of the present invention have variances in the sequence. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

It is contemplated that modified version of the immunogenic peptides of the present invention can be used, for example, peptides with amino acids substitutions, analogues, homologues, variants and peptides with substantial similarity to the immunogenic peptides, for example but not limited to immunogenic peptides corresponding to SEQ ID NOs 1 to 8.

For example in one embodiment, the peptide immunogens as disclosed herein, such as SEQ ID NO:1-8 or fragments, variants or derivatives thereof can be a retro-inverso peptide immunogens. A “retro-inverso peptide” refers to a peptide with a reversal of the direction of the peptide bond on at least one position, i.e., a reversal of the amino- and carboxy-termini with respect to the side chain of the amino acid. Thus, a retro-inverso analogue has reversed termini and reversed direction of peptide bonds while approximately maintaining the topology of the side chains as in the native peptide sequence. The retro-inverso peptide can contain L-amino acids or D-amino acids, or a mixture of L-amino acids and D-amino acids, up to all of the amino acids being the D-isomer. Partial retro-inverso peptide analogues are polypeptides in which only part of the sequence is reversed and replaced with enantiomeric amino acid residues. Since the retro-inverted portion of such an analogue has reversed amino and carboxyl termini, the amino acid residues flanking the retro-inverted portion are replaced by side-chain-analogous α-substituted geminal-diaminomethanes and malonates, respectively. Retro-inverso forms of cell penetrating peptides have been found to work as efficiently in translocating across a membrane as the natural forms. Synthesis of retro-inverso peptide analogues are described in Bonelli, F. et al., Int J Pept Protein Res. 24(6):553-6 (1984); Verdini, A. and Viscomi, G. C., J. Chem. Soc. Perkin Trans. 1:697-701 (1985); and U.S. Pat. No. 6,261,569, which are incorporated herein in their entirety by reference. Processes for the solid-phase synthesis of partial retro-inverso peptide analogues have been described (EP 97994-B) which is also incorporated herein in its entirety by reference.

As used herein, the term “substantial similarity” in the context of polypeptide sequences, indicates that the polypeptide comprises a sequence with at least 60% sequence identity to a reference sequence, or 70%, or 80%, or 85% sequence identity to the reference sequence, or most preferably 90% identity over a comparison window of about 10-20 amino acid residues. In the context of amino acid sequences, “substantial similarity” further includes conservative substitutions of amino acids. Thus, a polypeptide is substantially similar to a second polypeptide, for example, where the two peptides differ by one or more conservative substitutions.

The term “functional derivative” or “mimetic” are used interchangeably, and refers to a compound which possess a biological activity (either functional or structural) that is substantially similar to a biological activity of the entity or molecule its is a functional derivative of. The term functional derivative is intended to include the fragments, variants, analogues or chemical derivatives of a molecule, such as functional fragments, functional variants, functional analogues or functional chemical derivative of the peptide immunogens as disclosed herein, for example peptide immunogens of SEQ ID NOs: 1-8.

As used herein, “variant” with reference to a polynucleotide or polypeptide, refers to a polynucleotide or polypeptide that may vary in primary, secondary, or tertiary structure, as compared to a reference polynucleotide or polypeptide, respectively (e.g., as compared to a wild-type polynucleotide or polypeptide). For example, the amino acid or nucleic acid sequence may contain a mutation or modification that differs from a reference amino acid or nucleic acid sequence. In some embodiments, an peptide immunogen polynucleotide variant may be a different isoform or polymorphism. Peptide immunogen variants can be naturally-occurring, synthetic, recombinant, or chemically modified polynucleotides or polypeptides isolated or generated using methods well known in the art. Changes in the polynucleotide sequence of the variant may be silent. That is, they may not alter the amino acids encoded by the polynucleotide or polypeptide. Where alterations are limited to silent changes of this type, a variant will encode a polypeptide with the same amino acid sequence as the reference. Alternatively, such changes in the polynucleotide sequence of the variant may alter the amino acid sequence of a polypeptide encoded by the reference polynucleotide, resulting in conservative or non-conservative amino acid changes, as described below. Such polynucleotide changes may result in amino acid substitutions, additions, deletions, fusions and truncations in the polypeptide encoded by the reference sequence. Various; codon substitutions, such as the silent changes that produce various restriction sites, may be introduced to optimize cloning into a plasmid or viral vector or expression in a particular prokaryotic or eukaryotic system.

A “variant” of an immunogenic peptide, for example a variant of SEQ ID NO:1 is meant to refer to a molecule substantially similar in structure and function to either the entire molecule, or to a fragment thereof. A molecule is said to be “substantially similar” to another molecule if both molecules have substantially similar structures or if both molecules possess a similar biological activity. Thus, provided that two molecules possess a similar activity, they are considered variants as that term is used herein even if the structure of one of the molecules not found in the other, or if the sequence of amino acid residues is not identical.

As used herein, a “fragment” of a peptide or molecule as used herein refers to any contiguous polypeptide subset of the molecule, that is a peptide that comprises a subset of amino acids in the initial peptide molecule. Fragments of an immunogenic peptide, for example a fragment of SEQ ID NO:1 is meant to refer to a peptide that is at least one peptide shorter than SEQ ID NO:1 which has the activity as SEQ ID NO:1 are also encompassed for use in the present invention. A fragment of an peptide immunogen as disclosed herein, for example a functional fragment of SEQ ID NOs: 1-8 useful in the methods and compositions as disclosed herein have at least 30% of agonist or antagonist activity as that of SEQ ID NOs: 1-8. Stated another way, a fragment of an peptide immunogen is a fragment of any of SEQ ID NOs: 1-8 which can result in at least 30% of the same activity as compared to SEQ ID NOs: 1-8 to induce a CTL response in vivo or in a population of T cells (as disclosed in the Examples 2 and 3). It can also include fragments that decrease the wild type activity of one property by at least 30%. Fragments as used herein are soluble (i.e. not membrane bound). A “fragment” can be at least about 5, at least about 6, least about 7, at least about 8, at least about 9, at least about 10, at least about 11 amino acids, at least about . . . 15 amino acids or more and all integers in between 5 and 15 amino acids. Exemplary fragments include C-terminal truncations, N-terminal truncations, or truncations of both C- and N-terminals (e.g., deletions of, for example, at least 1, at least 2, at least 3, at least 4, at least 5, at least 6, at least 7, at least 8, at least 9, at least 10, at least 15 or more amino acids deleted from the N-termini, the C-termini, or both). One of ordinary skill in the art can create such fragments by simple deletion analysis. Such a fragment of SEQ ID NOs: 1 to 8 can be, for example, 1, 2, 3, 4, 5, 6, 7, 8, 9 or 10 amino acids or more than 10 amino acids deleted from the N-terminal and/or C-terminal of SEQ ID NOs: 1 to 8, respectively. Persons of ordinary skill in the art can easily identify the minimal peptide fragment of SEQ ID NOs: 1 to 8 useful in the methods and compositions as disclosed herein, by sequentially deleting N- and/or C-terminal amino acids from SEQ ID NOs: 1 to 8 and assessing the function of the resulting peptide immunogen fragment. One can create functional fragments with multiple smaller fragments. These can be attached by bridging peptide linkers. One can readily select linkers to maintain wild type conformation. One of ordinary skill in the art can easily assess the function of a peptide immunogen fragment to induce a CTL response in vivo or in a population of T cells (as disclosed in the Examples 2 and 3) as compared to the peptide immunogen corresponding to SEQ ID NOs: 1-8 as disclosed herein. Using such an in vivo assay, if the peptide immunogen fragment has at least 30% of the biological activity of the peptide immunogen corresponding to SEQ ID NOs: 1-8 as disclosed herein, then the peptide immunogen fragment is considered a valid peptide immunogen fragment and can used in the methods and compositions as disclosed herein. In some embodiments, a fragment of SEQ ID NOS: 1 to 8 can be less than 9, or less than 8 or less than 7, or less than 6, or less than 5 amino acids of SEQ ID NOS: 1 to 8. However, as stated above, the fragment must be at least about 5, at least about 6, least about 7, at least about 8, at least about 9, at least about 10, at least about 11 amino acids or more or any integers in between. In some embodiments, a preferred fragment is about 5 or 6 amino acids in length. As discussed above, a peptide immunogen variant can comprise a peptide immunogen fragment and additional amino acids added to one or both C- or N-terminal ends of the peptide immunogen fragment.

An “analog” of a molecule such an immunogenic peptide, for example an analogue of SEQ ID NO:1 is meant to refer to a molecule substantially similar in function to either the entire molecule or to a fragment thereof. As used herein, a molecule is said to be a “chemical derivative” of another molecule when it contains additional chemical moieties not normally a part of the molecule. Such moieties can improve the molecule's solubility, absorption, biological half life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., MackPubl., Easton, Pa. (1990).

The term “substitution” when referring to an immunogenic peptide, refers to a change in an amino acid for a different entity, for example another amino acid or amino-acid moiety. Substitutions can be conservative or non-conservative substitutions.

The term “conservative substitution,” when describing an immunogenic peptide, refers to a change in the amino acid composition of the polypeptide that does not substantially alter the polypeptide's activity, fore examples, a conservative substitution refers to substituting an amino acid residue for a different amino acid residue that has similar chemical properties. Conservative amino acid substitutions include replacement of a leucine with an isoleucine or valine, an aspartate with a glutamate, or a threonine with a serine. “Conservative amino acid substitutions” result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not substantially alter activity. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W.H. Freeman and Company (1984).) In addition, individual substitutions, deletions or additions that alter, add or delete a single amino acid or a small percentage of amino acids in an encoded sequence are also “conservative substitutions.” Insertions or deletions are typically in the range of about 1 to 5 amino acids.

As used herein, the term “non-conservative” refers to substituting an amino acid residue for a different amino acid residue that has different chemical properties. The nonconservative substitutions include, but are not limited to aspartic acid (D) being replaced with glycine (G); asparagine (N) being replaced with lysine (K); or alanine (A) being replaced with arginine (R).

As used herein, “insertions” or “deletions” are typically in the range of about 1 to 5 amino acids. The variation allowed may be experimentally determined by producing the peptide synthetically while systematically making insertions, deletions, or substitutions of nucleotides in the sequence using recombinant DNA techniques.

As used herein, the term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T. C, G. U. or 1) or residue occurs in both sequences to yield the number of matched positions, dividing the number of matched positions by the total number of positions in the comparison window (i.e., the window size), and multiplying the result by 100 to yield the percentage of sequence identity.

The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the; percentage of sequence identity is calculated by comparing the reference sequence to the sequence which may include deletions or additions which total 20 percent or less of the reference sequence over the comparison window. The reference sequence may be a subset of a larger sequence. The term “similarity”, when used to describe a polypeptide, is determined by comparing the amino acid sequence and the conserved amino acid substitutes of one polypeptide to the sequence of a second polypeptide.

Determination of homologs of the genes or peptides of the present invention may be easily ascertained by the skilled artisan. The terms “homology” or “identity” or “similarity” are used interchangeably herein and refers to sequence similarity between two peptides or between two nucleic acid molecules. Homology and identity can each be determined by comparing a position in each sequence which can be aligned for purposes of comparison. For example, it is based upon using a standard homology software in the default position, such as BLAST, version 2.2.14. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences, respectfully. A sequence which is “unrelated” or “non-homologous” shares less than 40% identity, though preferably less than 25% identity with a sequence of the present application.

In one embodiment, the term “immunogenic peptide homolog” to a protein identified as associated with the reference immunogenic peptide amino acid sequence refers to an amino acid sequence that has 40% homology to the full length amino acid sequence of the protein identified as associated with the reference immunogenic peptide, for example but not limited to at least 40% homology corresponding to SEQ ID NO: 1 of the present invention, or at least about 50%, still more preferably, at least about 60% homology, still more preferably, at least about 70% homology, even more preferably, at least about 75% homology, yet more preferably, at least about 80% homology, even more preferably at least about 85% homology, still more preferably, at least about 90% homology, and more preferably, at least about 95% homology. As discussed above, the homology is at least about 50% to 100% and all intervals in between (i.e., 55%, 60%, 70%, 75%, 80%, 85%, 90%, 95%, 98%, etc.). Also included in the term immunogen peptide homologue are peptide immunogen variants which comprise a peptide immunogen fragment and additional amino acids added to one or both C- or N-terminal ends of the peptide immunogen fragment.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins and Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, such as version 2.2.14 with default parameters for an alignment which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra). One example of algorithm that is suitable for determining percent sequence identify and sequence similarity is the BLAST algorithm, which is described in Altschul et al., J. Mol. Biol. 215:403-410 (1990). Software for performing BLAST analyses is publicly available through the National Center or Biotechnology Information (http://www.ncbi.nlm.nih.gov/). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff& Henikoff, Proc. Natl. Acad. Sci. USA 89, 10915 (1989)).

For purposes of classifying amino acids substitutions as conservative or nonconservative, amino acids are grouped as follows: Group I (hydrophobic sidechains): norleucine, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

In some embodiments, the immunogenic peptides of the present invention are typically substantially pure. This means that an immunogenic peptide is typically at least about 50% w/w (weight/weight) purity, as well as being substantially free from interfering proteins and contaminants. Sometimes the immunogenic peptide are at least about 80% w/w and, more preferably at least 90 or about 95% w/w purity. However, using conventional protein purification techniques, homogeneous peptides of at least 99% w/w can be obtained.

Immunogenic peptides or peptide immunogens of the present invention, its fragments, analogs and variants can be synthesized by solid phase peptide synthesis or recombinant expression, or can be obtained from natural sources according to standard methods well known in the art. Additionally, other compositions, methods of extracting and determining sequences are known in the art available to persons desiring to make and use such compositions. Automatic peptide synthesizers may be used to make such compositions and are commercially available from numerous manufacturers, such as Applied Biosystems (Perkin Elmer; Foster City, Calif.), and procedures for preparing synthetic peptides are known in the art. Recombinant expression can be in bacteria, such as E. coli, yeast, insect cells or mammalian cells; alternatively, proteins can be produced using cell free in vitro translation systems known in the art. Procedures for recombinant expression are described by Sambrook et al., Molecular Cloning: A Laboratory Manual (C.S.H.P. Press, NY 2d ed., 1989).

In some embodiments, the peptide immunogens also include longer polypeptides that include, for example, an immunogenic lambda 6(2-10) peptide, active fragment or analog together with other amino acids. Such polypeptides can be screened for prophylactic or therapeutic efficacy in animal models as described below. The immunogenic peptide of the present invention, for example immunogenic lambda 6(2-10) peptide, analog, active fragment or other polypeptide can be administered in associated form or in dissociated form. The immunogenic peptides of the present invention also include multimers of monomeric immunogenic peptides, as well as peptide immunogens conjugates, for example peptide immunogens conjugated to other antigens, or carrier proteins for increased immunogenicity of the peptide immunogen.

### V. Plasma Cell Malignancies

One aspect of the present invention relate to methods for the treatment of subjects with, or at risk of developing, plasma cell disorder or malignancies. In some embodiments, the plasma cell disorder is plasma cell dyscrasias (PCDs). In some embodiments, a plasma cell dyscrasias is AL amyloidosis. In alternative embodiments, the plasma cell disorder or malignancy is multiple myeloma. In other embodiments, the plasma cell disorder or malignancy is AL amyloidosis, AL amyloid, monoclonal gammopathies, multiple myeloma, MGUS (monoclonal gammopathy of undetermined significance) and Waldenstrom's macrogloblinema.

It is a general discovery of the present invention that plasma cell malignancies can be treated by administering the immunogenic peptides as disclosed herein to stimulate an immune response against a the plasma cells or immunoglobulin producing-plasma cell in the subject. The examples of plasma cell malignancies which can be treated with the methods and compositions as disclosed herein are discussed below and serve only to exemplify major forms of plasma cell malignancies and to identify subjects amenable to treatment by the methods using the peptide immunogens as disclosed herein and are not in any way intended to limit the invention.

Plasma cell dyscrasias (PCDs), or monoclonal gammopathy, are disorders characterized by the disproportionate proliferation of one clone of cells normally engaged in immunoglobulin (Ig) synthesis, and the presence of a structurally and electrophoretically homogeneous IG or polypeptide subunit in serum or urine. The disorders may be primarily asymptomatic to progressive, overt neoplasms (e.g., multiple myeloma). The disorder results from disproportionate proliferation of one clone producing a specific Ig: IgG, IgM, IgA, IgD or IgE.

### A. AL Amyloidosis

AL amyloid deposition is generally associated with almost any dyscrasia of the B lymphocyte lineage, ranging from malignancy of plasma cells (multiple myeloma) to benign monoclonal gammopathy. At times, the presence of amyloid deposits may be a primary indicator of the underlying dyscrasia.

Without being bound by theory, in AL-amyloidosis, fibrils of AL amyloid deposits are composed of monoclonal immunoglobulin light chains or fragments thereof. More specifically, the fragments are a region of the N-terminal region of the light chain (kappa or lambda), or derivatives thereof, and contain all or part of the variable (VL) domain thereof. More specifically, the fragments do not contain a region of the heavy chain of the variable region (VH). Deposits generally occur in the mesenchymal tissues, causing peripheral and autonomic neuropathy, carpal tunnel syndrome, macroglossia, restrictive cardiomyopathy, arthropathy of large joints, immune dyscrasias, multiple myelomas, as well as ocular dyscrasias. However, it should be noted that almost any tissue, particularly visceral organs such as the heart, may be involved.

In light chain amyloidosis (AL-amyloidosis) a monoclonal immunoglobulin light chain forms the amyloid deposits. See Glenner et al., Amyloid Fibril Proteins: Proof of Homology with Immunoglobulin Light Chains by Sequence Analyses, Science 172:1150-1151, 1971. Amyloid fibrils from patients suffering AL-amyloidosis occasionally contain only intact light chains, but more often they are formed by proteolytic fragments of the light chains which contain the VL domain and varying amounts of the constant domain, or by a mixture of fragments and full-length light chains. Not all light chains from plasma cell dyscrasias form protein deposits; some circulate throughout the body at high concentrations and are excreted with the subject's urine without pathological deposition of the protein in vivo. See Solomon, Clinical Implications of Monoclonal Light Chains, Semin. Oncol. 13:341-349, 1986; Buxbaum, Mechanisms of Disease: Monoclonal Immunoglobulin Deposition, Amyloidosis, Light Chain Deposition Disease, and Light and Heavy Chain Deposition Disease, Hematol./Oncol. Clinics of North America 6:323-346, 1992; and Eulitz, Amyloid Formation from Immunoglobulin Chains, Biol. Chef Hoppe-Seyler 373:629-633, 1992. Subjects suffering from AL amyloidosis can be recognized from methods known by a physician of ordinary skill, for example, typical symptoms of amyloidosis depend on the organ affected and include a wide range of symptoms, for example but are not limited to at least one of the following or combinations of; swelling of your ankles and legs, weakness, weight loss, shortness of breath, numbness or tingling in your hands or feet, diarrhea, severe fatigue, an enlarged tongue (macroglossia), skin changes, an irregular heartbeat, and difficulty swallowing. In some instances, the subject may not experience any of the symptoms listed but still has amyloidosis. In addition, a number of diagnostic tests are available for identifying subjects at risk of, or having AL amyloidosis which are commonly known by person skilled in the art, and are encompassed for use in the present invention. These include measurement of including blood and urine tests, though blood or urine tests may detect an abnormal protein, which could indicate amyloidosis, the only definitive test for amyloidosis is a tissue biopsy, in which the physicial analyses a small sample of tissue. The tissue sample may be taken from one or more parts of the subject's body, for example abdominal fat, bone marrow or rectum, which is then examined under a microscope in a laboratory to check for signs of amyloid. Occasionally, tissue samples may be taken from other parts of your body, such as your liver or kidney, to help diagnose the specific organ affected by amyloidosis.

The methods of the present invention are useful to treat the main forms of amyloidosis and AL amyloidosis, for example primary amyloidosis, secondary amyloidosis and hereditary amyloidosis.

Primary amyloidosis. This most common form of amyloidosis primarily affects your heart, kidneys, tongue, nerves and intestines. Primary amyloidosis isn't associated with other diseases except for multiple myeloma, in a minority of cases. The cause of primary amyloidosis is unknown, but doctors do know that the disease begins in your bone marrow. In addition to producing red and white blood cells and platelets, your bone marrow makes antibodies, the proteins that protect you against infection and disease. After antibodies serve their function, your body breaks them down and recycles them. Amyloidosis occurs when cells in the bone marrow produce antibodies that can't be broken down. These antibodies then build up in your bloodstream. Ultimately, they leave your bloodstream and can deposit in your tissues as amyloid, interfering with normal function.

Secondary amyloidosis. This form occurs in association with chronic infectious or inflammatory diseases, such as tuberculosis, rheumatoid arthritis or osteomyelitis, a bone infection. It primarily affects your kidneys, spleen, liver and lymph nodes, though other organs may be involved. Treatment of the underlying disease may help stop this form of amyloidosis.

Hereditary amyloidosis. As the name implies, this form of amyloidosis is inherited. This type often affects the nerves, heart and kidneys.

### B. Multiple Myeloma

Multiple myeloma (MM), also known as plasma cell multiple myeloma or multiple myelomatosis, is a progressive neoplastic disease characterized by marrow plasma cell tumors and overproduction of an intact monoclonal Ig (IgG, IgA, IgD or IgE) or Bence Jones protein, which is free monoclonal kappa or lambda light chains. Diffuse osteoporosis or discrete osteolytic lesions arise due to replacement by expanding plasma cell tumors or an osteoclast-activating factor secreted by malignant plasma cells.

Specific criteria exist that serve as useful guidelines to allow clinicians to differentiate between MM and other multiple myeloma-related plasma proliferative disorders. Multiple myeloma-related plasmaproliferative disorders are defined to include MGUS, smoldering MM (SMM) and indolent MM (IMM). Subjects with MGUS, a clinically benign condition, usually have less than 10% marrow plasma cells, a serum monoclonal protein <3 gm/dL, no urinary Bence Jones protein and no anemia, renal failure, lytic bone lesions, or hypercalcemia. In contrast, patients with active MM present with a marrow plasmacytosis of greater than or equal to 10%, a serum monoclonal protein of greater than or equal to 3 gm/dL, a 24-hour urine monoclonal protein of greater than or equal to 1 gm, and lytic bone lesions. MM patients also often present with back pain, severe fatigue, pneumonia, or bone pain. Kyle and Lust (1989) Seminars in Hematology 26:176. Patients with SMM are usually asymptomatic. They have a marrow plasmacytosis of greater than or equal to 10% and/or a serum monoclonal protein of [greater than or equal to] 3 gm/dL. Lytic bone lesions are absent and they have stable disease. See Kyle and Greipp (1980) N. Engl. J. Med. 302:1347. Patients with IMM are similar to those with SMM, except bone lesions may be present on bone survey studies. In contrast to MM patients, who all receive chemotherapy, patients with MGUS, SMM and IMM have stable disease and are followed off chemotherapy. However, patients who have a serum monoclonal protein of >2.0 g/dL are at high risk of eventually developing active MM, requiring chemotherapy. Such patients are candidates for novel therapeutic strategies to inhibit or prevent the development of active MM.

### C. Gammopathies

Macroglobulinemia, or primary or Waldenstrom's macroglobulinemia, is a plasma cell dyscrasia involving B cells that normally synthesize and secrete IgM. Macrogolbulinemia is distinct from muliple multiple myeloma and other PCDs, and resembles a lymphomatous disease. Many patients have symptoms of hyperviscosity; fatigue, weakness, skin and mucosal bleeding and so forth.

### D. Miscellaneous Amyloidoses

There are a variety of other forms of amyloid disease that are normally manifest as localized deposits of amyloid. In general, these diseases are probably the result of the localized production and/or lack of catabolism of specific fibril precursors or a predisposition of a particular tissue (such as the joint) for fibril deposition. Examples of such idiopathic deposition include nodular AL amyloid, cutaneous amyloid, endocrine amyloid, and tumor-related amyloid.

In some types of hereditary amyloidoses, single amino acid changes in normal human proteins are responsible for amyloid fibril formation See Natvig et al., Amyloid and Amyloidosis 1990. Dordrecht, The Netherlands: Kluwer Academic Publishers, 1991, and references cited therein. It is unlikely, however, that any single amino acid position or substitution will fully explain the many different immunoglobulin light chain sequences associated with AL-amyloidosis. Rather, several different regions of the light chain molecule may sustain one or more substitutions which affect a number of biophysical characteristics, such as dimer formation, exposure of hydrophobic residues, solubility, and stability.

Heavy chain diseases are neoplastic plasma cell dyscrasias characterized by the overproduction of homogenous α, γ, and mu Ig heavy chains. These disorders result in incomplete monoclonal Igs. The clinical picture is more like lymphoma than multiple myeloma.

### E. Malignancies and Cancers

In some embodiments, the methods of the present invention are useful for the treatment of cancers and other malignanices. For example, the present invention provides methods useful in the treatment of cancers, further cancers associated with proliferation of a cell which secretes specific peptides or protein. Any cancer that secretes a peptide or protein can be treated by the methods of the present invention. Exemplary cancer that secretes proteins are hormone-secreting tumors, which may also contain hormone-derived amyloid fibrils and plaques. Such fibrils may be made up of polypeptide hormones such as calcitonin (medullary carcinoma of the thyroid), islet amyloid polypeptide (occurring in most patients with Type II diabetes), and atrial natriuretic peptide (isolated atrial amyloidosis). In such embodiments, the immunogenic peptide for use in the methods of the present invention is substantially similar to a region of the a hormone that is secreted by the tumor or cancer, for example but not limited to, calcitonin, islet amyloid polypeptide and atrial natriuretic peptide. The immunogenic peptides of the present invention include peptides to fragments of such hormones or their homologues or variants.

In some embodiments, the methods of the present invention are useful in the therapeutic treatment of subjects at risk of disease but not showing symptoms of the plasma cell disorder or malignancy or plasma cell dyscrasias, as well as subjects showing symptoms. Therefore, the pharmaceutical compositions comprising the immunogenic peptides of the present invention can be administered prophylactically without any assessment of the risk of the subject. The present methods are especially useful for individuals who do have a known genetic risk of plasma cell disorder or malignancy, for example AL amyloidosis or multiple myeloma or other amyloidosis-related disorder, such as for example Alzheimer's disease. Such subjects include those having relatives who have experienced this disease and those whose risk is determined by analysis of genetic or biochemical markers.

In asymptomatic subjects, treatment can begin at any age (e.g., 10, 20, 30). Treatment typically entails multiple dosages over a period of time. Treatment can be monitored by assaying antibody, or activated T-cell or B-cell responses to the therapeutic agent (e.g., the immunogenic peptide of the present invention) over time. If the response falls, a booster dosage is indicated. In the case of potential hereditary plasma cell maligancies, treatment can begin antenatally by administering therapeutic agent to the mother or shortly after birth.

### F. Amyloidogenic Diseases.

In some embodiments, the present invention provides methods to treat a disease and/or disorder associated with an amyloidogenic disease. Amyloidogenic diseases are diseases are diseases from the secretion of a protein and/or peptide that aggregates and forms a deposit and is characterized by amyloid deposits or fibril formation. The methods of the present invention provide methods to design and use of immunogenic peptides for the treatment of such amyloidogenic diseases or amyloid-related diseases. Such amyloidogenic diseases, or amyloid-related diseases include, for example but is not limited to, Alzheimer's disease, Down's syndrome, vascular dementia or cognitive impairment, type II diabetes mellitus, amyloid A (reactive), secondary amyloidosis, familial mediterranean fever, familial nephrology with urtcaria and deafness (Muckle-wells Syndrome), amyloid lambda L-chain or amyloid kappa L-chain (idiopathic, multiple myeloma or macroglobulinemia-associated) A beta 2M (chronic hemodialysis), ATTR (familial amyloid polyneuropathy (Portuguese, Japanese, Swedish), familial amyloid cardiomyopathy (Danish), isolated cardiac amyloid, (systemic senile amyloidosis) AIAPP or amylin insulinoma, atrial naturetic factor (isolated atrial amyloid), procalcitonin (medullary carcinoma of the thyroid), gelsolin (familial amyloidosis (Finnish), cyctatin C (heritiaty cerebral hemorrhage with amyloidosis (Icelandic), AApo-A-I (familial amyloidotic polyneuropathy—Iowa), AApo-A-II (accelerated senescence in mice), fibrinogen-associated amyloid; and Asor or Pr P-27 (scrapie, Creutzfeld jacob disease, Gertsmann-Straussler-Scheinker syndrome, bovine spongiform encephalitis) and person who are homozygous for the apolipoprotein E4 allele.

As used herein, the terms “treat,” “treating,” and “treatment” refer to the alleviation or measurable lessening of one or more symptoms or measurable markers of a disease or disorder; while not intending to be limited to such, disease or disorders of particular interest include ischemic or ischemia/reperfusion injury and diabetes. Measurable lessening includes any statistically significant decline in a measurable marker or symptom.

As used herein, the terms “prevent,” “preventing” and “prevention” refer to the avoidance or delay in manifestation of one or more symptoms or measurable markers of a disease or disorder. A delay in the manifestation of a symptom or marker is a delay relative to the time at which such symptom or marker manifests in a control or untreated subject with a similar likelihood or susceptibility of developing the disease or disorder. The terms “prevent,” “preventing” and “prevention” include not only the complete avoidance or prevention of symptoms or markers, but also a reduced severity or degree of any one of those symptoms or markers, relative to those symptoms or markers arising in a control or non-treated individual with a similar likelihood or susceptibility of developing the disease or disorder, or relative to symptoms or markers likely to arise based on historical or statistical measures of populations affected by the disease or disorder. By “reduced severity” is meant at least a 10% reduction in the severity or degree of a symptom or measurable disease marker, relative to a control or reference, e.g., at least 15%, 20%, 30%, 40%, 50%, 60%, 70%, 80%, 90%, 95%, 99% or even 100% (i.e., no symptoms or measurable markers).

### VI. Delivery Mechanisms

In a further variation, an immunogenic peptide of the present invention, for example lambda 6(2-10) peptide or heteroclitic lambda 6(2-10) peptide, can be presented as a viral or bacterial vaccine as part of the pharmaceutical composition. A nucleic acid encoding the immunogenic peptide is incorporated into a genome or episome of the virus or bacteria. Optionally, the nucleic acid is incorporated in such a manner that the immunogenic peptide is expressed as a secreted protein or as a fusion protein with an outer-surface protein of a virus or a transmembrane protein of a bacteria so that the peptide is displayed. Viruses or bacteria used in such methods should be nonpathogenic or attenuated. Suitable viruses include adenovirus, HSV, Venezuelan equine encephalitis virus and other alpha viruses, vesicular stomatitis virus, and other rhabdo viruses, vaccinia and fowl pox. Suitable bacteria include Salmonella and Shigella. Fusion of an immunogenic peptide to a helper peptide, for example HBsAg of HBV is also encompassed for use in the present invention.

In some embodiments, pharmaceutical compositions comprising the immunogenic peptides of the present invention may also optionally comprise other peptides and other compounds that do not necessarily have a significant amino acid sequence similarity with immunogenic peptides of the present invention but nevertheless serve as mimetics of lambda light and induce a similar immune response.

Random libraries of peptides or other compounds can also be screened for suitability as mimetics or functional derivatives of the immunogenic peptides as disclosed herein. Combinatorial libraries can be produced for many types of compounds that can be synthesized in a step-by-step fashion. Such compounds include polypeptides, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described in Affymax, WO 95/12608, Affymax, WO 93/06121, Columbia University, WO 94/08051, Pharmacopeia, WO 95/35503 and Scripps, WO 95/30642 (each of which is incorporated by reference for all purposes). Peptide libraries can also be generated by phage display methods. See, e.g., Devlin, WO 91/18980.

The pharmaceutical compositions of the invention also include antibodies that specifically bind to immunoglobulin light chain lambda, for example antibodies that bind to the lambda 6 immunoglobulin light chain. Such antibodies are thus termed anti-idiotype or anti-idiotypic antibodies. Such anti-Id antibodies mimic the antigen, for example they mimic a region of the lambda 6 light chain and generate an immune response to it (see Essential Immunology (Roit ed., Blackwell Scientific Publications, Palo Alto, 6th ed.), p. 181).

### VII. Immunogenic Peptides for Passive Immune Response

In the present invention, the immunogenic peptides of the present invention can trigger the production of anti-idiotypic antibodies against immunoglobulins and monoclonal antibodies, for example immunoglobulins produced by plasma cells and B cells. Use of such anti-idiotypic antibodies is also encompassed for use in the present invention, for example, for use in a pharmaceutical composition for example to treat a subject with a plasma cell disorder or malignancy or cancer. Administration of such an antibody is known as passive immunotherapy or passive immunization by persons of ordinary skill in the art, and any means to generate antibodies directed to a peptide immunogen of the present invention are commonly known in the art and are useful in the methods of the present invention. As used herein, the term “directed to” with respect to an “antibody directed to” is intended to refer to an antibody that specifically recognizes the immunogen of the present invention or an antibody that recognizes a protein comprising a region substantially similar to the peptide immunogen of the present invention. For example, such antibodies directed to the peptides immunogens can be generated using the peptide immunogen as the epitope for antibody selectivity. In some embodiments, the antibodies can be monoclonal or polyclonal. In some embodiments, the antibodies bind specifically to the immunoglobulin light chain on the surface of the plasma cell or B cell. In alternative embodiments, the antibodies bind to the protein comprising a region substantially similar to the peptide immunogen of the present invention expressed on the surface of a cell, for example a tumor cell or a cell associated with an amyloidogenic disease.

Also encompassed in some embodiments of the present invention is administration to a subject or antibody directed to the peptide immunogen of the present invention to treat a subject with a plasma cell disorder or malignancy, for example a plasma cell dyscrasias. In alternative embodiments, the methods also provide for treating disorders such as cancers with an antibody directed to a peptide immunogen of the present invention, where the antibody specifically bind to a peptide on the surface of a cancer cell or tumor cell.

In some embodiments, the antibodies directed to the peptide immunogens of the present invention are administered in a pharmaceutical composition. In some embodiments, the antibodies are conjugated or tagged with a toxin to enhance the killing effect on the cell. Such toxins are commonly known in the art and commonly used for immunotherapy and are encompassed for use in the methods of the present invention.

In some embodiments, the pharmaceutical composition of the present invention also include T-cells that bind to the immunogenic peptides of the present invention. For example, T-cells can be activated against the immunogenic peptides of the present invention, for example but not limited to lambda 6₍₂₋₁₀₎ peptide by expressing a human MHC class I gene and a human β-2-microglobulin gene from an insect cell line, whereby an empty complex is formed on the surface of the cells and can bind to the lambda 6₍₂₋₁₀₎ peptide. T-cells contacted with the cell line become specifically activated against the peptide. See Peterson et al., U.S. Pat. No. 5,314,813. Insect cell lines expressing an MHC class II antigen can similarly be used to activate CD4⁺ T cells.

The production of non-human monoclonal antibodies, e.g., murine or rat, can be accomplished by, for example, immunizing the animal with an immunogenic peptide of the present invention, for example but not limited to lambda 6₍₂₋₁₀₎ or hc lambda 6₍₂₋₁₀₎. See Harlow & Lane, Antibodies, A Laboratory Manual (CSHP NY, 1988) (incorporated by reference for all purposes). Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861 (incorporated by reference for all purposes).

Human antibodies can be obtained using phage-display methods. See, e.g., Dower et al., WO 91/17271; McCafferty et al., WO 92/01047. In these methods, libraries of phage are produced in which members display different antibodies on their outersurfaces. Antibodies are usually displayed as Fv or Fab fragments. Phage displaying antibodies with a desired specificity are selected by affinity enrichment to immunoglobulin lambda 6 light chain or fragments thereof. Human antibodies against immunoglobulin lambda 6 light chain can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991) (each of which is incorporated by reference in its entirety for all purposes). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using a region of the immunoglobulin lambda light chain, for example a region of the lambda 6 light chain, or other lambda light chain peptides as an affinity reagent.

Human or humanized antibodies can be designed to have IgG, IgD, IgA and IgE constant region, and any isotype, including IgG1, IgG2, IgG3 and IgG4. Antibodies can be expressed as tetramers containing two light and two heavy chains, as separate heavy chains, light chains, as Fab, Fab′F(ab′)₂, and Fv, or as single chain antibodies in which heavy and light chain variable domains are linked through a spacer.

a. Production of Non-Human Antibodies. The production of non-human monoclonal antibodies, e.g., murine, guinea pig, rabbit or rat, can be accomplished by, for example, immunizing the animal with an immunogenic peptides of the present invention, for example but not limited to a peptide with any of SEQ ID NO: 1-8. Any immunogenic peptide substantially similar to a region of the lambda 1, 2, 3 or 6 is encompassed for use, for example immunogenic peptides substantially similar to a region of the lambda 6, e.g. the lamda 6(2-10) peptide or a longer peptide thereof, can be used. See e.g., Harlow Lane, Antibodies, A Laboratory Manual (CSHP NY, 1988) (incorporated by reference for all purposes). Such immunogenic peptides can be obtained from a natural source, by peptide synthesis or by recombinant expression. Optionally, immunogenic peptides can be administered fused or otherwise complexed with a carrier protein, as described herein. Optionally, immunogenic peptides can be administered with an adjuvant. Several types of adjuvant can be used as described herein. Complete Freund's adjuvant followed by incomplete adjuvant is preferred for immunization of laboratory animals. Rabbits or guinea pigs are typically used for making polyclonal antibodies. Mice are typically used for making monoclonal antibodies. Antibodies are screened for specific binding to the immunogen. Optionally, antibodies are further screened for binding to a specific region of the immunogen, for example the lambda light chain of an immunoglobulin. Binding can be assessed, for example, by Western blot or ELISA. The smallest fragment to show specific binding to the antibody defines the epitope of the antibody. Alternatively, epitope specificity can be determined by a competition assay is which a test and reference antibody compete for binding to the component. If the test and reference antibodies compete, then they bind to the same epitope or epitopes sufficiently proximal that binding of one antibody interferes with binding of the other.

b. Chimeric and Humanized Antibodies. Chimeric and humanized antibodies have the same or similar binding specificity and affinity as a mouse or other nonhuman antibody that provides the starting material for construction of a chimeric or humanized antibody. Chimeric antibodies are antibodies whose light and heavy chain genes have been constructed, typically by genetic engineering, from immunoglobulin gene segments belonging to different species. For example, the variable (V) segments of the genes from a mouse monoclonal antibody may be joined to human constant (C) segments, such as IgG1 and IgG4. A typical chimeric antibody is thus a hybrid protein consisting of the V or antigen-binding domain from a mouse antibody and the C or effector domain from a human antibody.

Humanized antibodies have variable region framework residues substantially from a human antibody (termed an acceptor antibody) and complementarity determining regions substantially from a mouse-antibody, (referred to as the donor immunoglobulin). See, Queen et al., Proc. Natl. Acad. Sci. USA 86:10029-10033 (1989) and WO 90/07861, U.S. Pat. No. 5,693,762, U.S. Pat. No. 5,693,761, U.S. Pat. No. 5,585,089, U.S. Pat. No. 5,530,101 and Winter, U.S. Pat. No. 5,225,539 (incorporated by reference in their entirety for all purposes). The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be substantially similar to a region of the same or different human antibody sequences. The human antibody sequences can be the sequences of naturally occurring human antibodies or can be consensus sequences of several human antibodies. See Carter et al., WO 92/22653. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid should usually be substituted by the equivalent framework amino acid from the mouse antibody when it is reasonably expected that the amino acid: (1) noncovalently binds antigen directly, (2) is adjacent to a CDR region, (3) otherwise interacts with a CDR region (e.g. is within about 6 A of a CDR region), or (4) participates in the VL-VH interface.

Other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins. Other candidates for substitution are acceptor human framework amino acids that are unusual for a human immunoglobulin at that position. The variable region frameworks of humanized immunoglobulins usually show at least 85% sequence identity to a human variable region framework sequence or consensus of such sequences.

c. Human Antibodies. Human antibodies against Ax3b2 are provided by a variety of techniques described below. Some human antibodies are selected by competitive binding experiments, or otherwise, to have the same epitope specificity as a particular mouse antibody. Human antibodies can also be screened for a particular epitope specificity by using only an immunogenic peptides of the present invention as the immunogen, and/or by screening antibodies for ability to kill plasma cells, as described in the examples.

(1) Trioma Methodology. The basic approach and an exemplary cell fusion partner, SPAZ-4, for use in this approach have been described by Oestberg et al., Hybridoma 2:361-367 (1983); Oestberg, U.S. Pat. No. 4,634,664; and Engleman et al., U.S. Pat. No. 4,634,666 (each of which is incorporated by reference in its entirety for all purposes). The antibody-producing cell lines obtained by this method are called triomas, because they are descended from three cells—two human and one mouse. Initially, a mouse multiple myeloma line is fused with a human B-lymphocyte to obtain a non-antibody-producing xenogeneic hybrid cell, such as the SPAZ-4 cell line described by Oestberg, supra. The xenogeneic cell is then fused with an immunized human B-lymphocyte to obtain an antibody-producing trioma cell line. Triomas have been found to produce antibody more stably than ordinary hybridomas made from human cells.

The immunized B-lymphocytes are obtained from the blood, spleen, lymph nodes or bone marrow of a human donor. If antibodies against a specific antigen or epitope are desired, it is preferable to use that antigen or epitope thereof for immunization. Immunization can be either in vivo or in vitro. For in vivo immunization, B cells are typically isolated from a human immunized with the immunogenic peptides of the present invention, for example a lambda 6₍₂₋₁₀₎ peptide. In some methods, B cells are isolated from the same patient who is ultimately to be administered antibody therapy. For in vitro immunization, B-lymphocytes are typically exposed to antigen for a period of 7-14 days in a media such as RPMI-1640 (see Engleman, supra) supplemented with 10% human plasma.

The immunized B-lymphocytes are fused to a xenogeneic hybrid cell such as SPAZ-4 by well known methods. For example, the cells are treated with 40-50% polyethylene glycol of MW 1000-4000, at about 37 degrees C., for about 5-10 min. Cells are separated from the fusion mixture and propagated in media selective for the desired hybrids (e.g., HAT or AH). Clones secreting antibodies having the required binding specificity are identified by assaying the trioma culture medium for the ability to bind to the lambda 6₍₂₋₁₀₎ peptide or a fragment thereof. Triomas producing human antibodies having the desired specificity are subcloned by the limiting dilution technique and grown in vitro in culture medium. The trioma cell lines obtained are then tested for the ability to bind to the lambda 6₍₂₋₁₀₎peptide or a fragment thereof.

Although triomas are genetically stable they do not produce antibodies at very high levels. Expression levels can be increased by cloning antibody genes from the trioma into one or more expression vectors, and transforming the vector into standard mammalian, bacterial or yeast cell lines, according to methods well known in the art.

(2) Transgenic Non-Human Mammals. Human antibodies against immunoglobulin light chains can also be produced from non-human transgenic mammals having transgenes encoding at least a segment of the human immunoglobulin locus. Usually, the endogenous immunoglobulin locus of such transgenic mammals is functionally inactivated. Preferably, the segment of the human immunoglobulin locus includes unrearranged sequences of heavy and light chain components. Both inactivation of endogenous immunoglobulin genes and introduction of exogenous immunoglobulin genes can be achieved by targeted homologous recombination, or by introduction of YAC chromosomes. The transgenic mammals resulting from this process are capable of functionally rearranging the immunoglobulin component sequences, and expressing a repertoire of antibodies of various isotypes encoded by human immunoglobulin genes, without expressing endogenous immunoglobulin genes. The production and properties of mammals having these properties are described in detail by, e.g., Lonberg et al., WO93/12227 (1993); U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741 (1991) (each of which is incorporated by reference in its entirety for all purposes). Transgenic mice are particularly suitable in this regard. Monoclonal antibodies are prepared by, e.g., fusing B-cells from such mammals to suitable multiple myeloma cell lines using conventional Kohler-Milstein technology. Human polyclonal antibodies can also be provided in the form of serum from humans immunized with an immunogenic agent.

(3) Phage Display Methods. A further approach for obtaining anti-immunglobulin light chains antibodies, for example anti-lambda6 containing immunoglobulin antibodies is to screen a DNA library front human B cells according to the general protocol outlined by Huse et al., Science 246:1275-1281 (1989). For example, as described for trioma methodology, such B cells can be obtained from a human immunized with the immunogenic peptides of the present invention, for example a lambda 6₍₂₋₁₀₎ peptide. Optionally, such B cells are obtained from a patient who is ultimately to receive antibody treatment. Antibodies binding to an epitope of the immunoglobulin light chain, for example immuoglobulins comprising lambda 6 light chains are selected. Sequences encoding such antibodies (or binding fragments) are then cloned and amplified. The protocol described by Huse is rendered more efficient in combination with phage-display technology. See, e.g., Dower et al., WO 91/17271 and McCafferty et al., WO 92/01047, U.S. Pat. No. 5,877,218, U.S. Pat. No. 5,871,907, U.S. Pat. No. 5,858,657, U.S. Pat. No. 5,837,242, U.S. Pat. No. 5,733,743 and U.S. Pat. No. 5,565,332 (each of which is incorporated by reference in its entirety for all purposes). In these methods, libraries of phage are produced in which members display different antibodies on their outer surfaces. Antibodies are usually displayed as Fv or Fab fragments. Phage displaying antibodies with a desired specificity are selected by affinity enrichment to immunoglobulins with lambda light chains, in particular immunglobulins with lambda 6 light chains.

In a variation of the phage-display method, human antibodies having the binding specificity of a selected murine antibody can be produced. See Winter, WO 92/20791. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions are obtained from a library of rearranged human heavy chain variable regions. A phage showing strong specific binding for the component of interest (e.g., at least 10⁸ and preferably at least 10⁹ M⁻¹) is selected. The human heavy chain variable region from this phage then serves as a starting material for constructing a further phage library. In this library, each phage displays the same heavy chain variable region (i.e., the region identified from the first display library) and a different light chain variable region. The light chain variable regions are obtained from a library of rearranged human variable light chain regions. Again, phage showing strong specific binding for amyloid peptide component are selected. These phage display the variable regions of completely human anti-amyloid peptide antibodies. These antibodies usually have the same or similar epitope specificity as the murine starting material.

d. Selection of Constant Region. The heavy and light chain variable regions of chimeric, humanized, or human antibodies can be linked to at least a portion of a human constant region. The choice of constant region depends, in part, whether antibody-dependent complement and/or cellular mediated toxicity is desired. For example, isotopes IgG1 and IgG3 have complement activity and isotypes IgG2 and IgG4 do not. Choice of isotype can also affect passage of antibody into the brain. Light chain constant regions can be lambda or kappa. Antibodies can be expressed as tetramers containing two light and two heavy chains, as separate heavy chains, light chains, as Fab, Fab F(ab)₂, and Fv, or as single chain antibodies in which heavy and light chain variable domains are linked through a spacer.

e. Expression of Recombinant Antibodies. Chimeric, humanized and human antibodies are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting antibodies.

These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences.

E. coli is one prokaryotic host particularly useful for cloning the DNA sequences of the present invention. Microbes, such as yeast are also useful for expression. Saccharomyces is a preferred yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells are a preferred host for expressing nucleotide segments encoding immunoglobulins or fragments thereof. See Winnacker, From Genes to Clones, (VCH Publishers, N.Y., 1987). A number of suitable host cell lines capable of secreting intact heterologous proteins have been developed in the art, and include CHO cell lines, various COS cell lines, HeLa cells, L cells and multiple myeloma cell lines. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters substantially similar to a region of the endogenous genes, cytomegalovirus, SV40, adenovirus, bovine papillomavirus, and the like. See Co et al., J. Immunol. 148:1149 (1992).

Alternatively, antibody coding sequences can be incorporated in transgenes for introduction into the genome of a transgenic animal and subsequent expression in the milk of the transgenic animal (e.g., according to methods described in U.S. Pat. No. 5,741,957, U.S. Pat. No. 5,304,489, U.S. Pat. No. 5,849,992, all incorporated by reference herein in their entireties). Suitable transgenes include coding sequences for light and/or heavy chains in operable linkage with a promoter and enhancer from a mammary gland specific gene, such as casein or beta lactoglobulin.

The vectors containing the DNA segments of interest can be transferred into the host cell by well-known methods, depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

Once expressed, antibodies can be purified according to standard procedures of the art, including HPLC purification, column chromatography, gel electrophoresis and the like (see generally, Scopes, Protein Purification (Springer-Verlag, NY, 1982)).

### VIII. Other Antigens and Carrier Proteins

Some immunogenic peptides useful in the present invention for inducing an immune response contain the appropriate epitope for inducing an immune response against the plasma deposits but are too small to be significantly immunogenic. In this situation, a peptide immunogen can be linked to another antigen or suitable carrier peptides to help elicit an immune response. Suitable antigens or carrier peptides include serum albumins, keyhole limpet hemocyanin, immunoglobulin molecules, thyroglobulin, ovalbumin, tetanus toxoid, or a toxoid from other pathogenic bacteria, such as diphtheria, E. coli, cholera, or H. pylori, or an attenuated toxin derivative. Other carriers for stimulating or enhancing an immune response include cytokines such as IL-1, IL-1 α and β peptides, IL-2, γINF, IL-10, GM-CSF, and chemokines, such as M1P1α and β and RANTES. Immunogenic agents can also be linked to peptides that enhance transport across tissues, as described in O'Mahony, WO 97/17613 and WO 97/17614, which are incorporated herein in their entirety by reference.

In some embodiments, the immunogenic peptide of the present invention can be linked to a suitable antigens or other carrier peptides to help elicit an immune response. Antigens useful in the methods of the present invention, are for example but are not limited to include T-cell epitopes that bind to multiple MHC alleles, e.g., at least 75% of all human MHC alleles. Such carriers are sometimes known in the art as “universal T-cell epitopes.” Examples of universal T-cell epitopes include: Influenza Hemagluttinin: HA[307-319]PKYVKQNTLKLAT (SEQ ID NO: 9); PADRE (common residues bolded) AKXVAAWTLKAAA (SEQ ID NO: 10); Malaria CS: T3 epitope EKKIAKMEKASSVFNV (SEQ ID NO: 11); Hepatitis B surface antigen: HBsAg[19-28] FFLLTRILTI (SEQ ID NO: 12); Heat Shock Protein 65: hsp65[153-171] DQSIGDLIAEAMDKVGNEG (SEQ ID NO: 13); bacille Calmette-Guerin QVHFQPLPPAVVKL (SEQ ID NO: 14); Tetanus toxoid: TT[830-844] QYIKANSKFIGITEL (SEQ ID NO: 15); Tetanus toxoid: TT[947-967]FNNFTVSFWLRVPKVSASHLE (SEQ ID NO: 16); HIV gp120 T1: KQIINMWQEVGKAMYA. (SEQ ID NO: 17); or homologues, derivatives or fragments thereof.

Immunogenic peptides of the present invention can be linked to carriers by chemical crosslinking. Techniques for linking an immunogen to a carrier include the formation of disulfide linkages using N-succinimidyl-3-(2-pyridyl-thio)propionate (SPDP) and succinimidyl 4-(N-maleimidomethyl)cyclohexane-1-carboxylate (SMCC) (if the peptide lacks a sulfhydryl group, this can be provided by addition of a cysteine residue). These reagents create a disulfide linkage between themselves and peptide cysteine resides on one protein and an amide linkage through the C-amino on a lysine, or other free amino group in other amino acids. A variety of such disulfide/amide-forming agents are described by Immun. Rev. 62, 185 (1982). Other bifunctional coupling agents form a thioether rather than a disulfide linkage. Many of these thio-ether-forming agents are commercially available and include reactive esters of 6-maleimidocaproic acid, 2-bromoacetic acid, and 2-iodoacetic acid, 4-(N-maleimidomethyl)cyclohexane-1-carboxylic acid. The carboxyl groups can be activated by combining them with succinimide or 1-hydroxyl-2-nitro-4-sulfonic acid, sodium salt.

Immunogenic peptides can also be expressed as fusion proteins with carriers. The immunogenic peptide can be linked at the amino terminus, the carboxyl terminus, or internally to the carrier. Optionally, multiple repeats of the immunogenic peptide can be present in the fusion protein. Optionally, an immunogenic peptide can be linked to multiple copies of a heterologous peptide, for example, at both the N and C termini of the peptide. Some carrier peptides serve to induce a helper T-cell response against the carrier peptide. The induced helper T-cells in turn induce a B-cell response against the immunogenic peptide linked to the carrier peptide.

In some embodiments, the immunogenic peptide of the present invention is linked to carrier peptides that function as targeting agents, for example to target the immunogenic peptide to a particular target cell or target organ. Such carrier peptides are well known by persons of ordinary skill in the art and are encompassed for use in the methods of the present invention.

### IX. Pharmaceutical Compositions

The present invention provides pharmaceutical compositions capable of eliciting or providing an immune response directed to certain proteins expressed by plasma cells, for example components of immunoglobulin light chains, which are effective to treat or prevent development of plasma cell malignancies, for example AL amyloidosis and multiple myeloma. In particular, according to the invention provided herein, it is possible to prevent progression of, ameliorate the symptoms of, and/or reduce plasma cell dyscrasias, disorder or malignancies in afflicted subjects, when an effective amount or effective dose of an pharmaceutical composition comprising an immunogenic peptide of the present invention is administered to the subject in need thereof.

In prophylactic applications, pharmaceutical compositions (or medicants) are administered to a subject susceptible to, or otherwise at risk of, a particular disease, for example a plasma cell dyscrasias or disorder in an amount sufficient to eliminate or reduce the risk or delay the outset of the disease. In therapeutic applications, pharmaceutical compositions (or medicants) are administered to a subject suspected of, or already suffering from such a disease in an amount sufficient to cure, or at least partially arrest, the symptoms of the disease and its complications. An amount adequate to accomplish this is defined as a therapeutically- or pharmaceutically-effective dose. In both prophylactic and therapeutic regimes, agents are usually administered in several dosages until a sufficient immune response has been achieved. Typically, the immune response is monitored and repeated dosages are given if the immune response starts to fade.

The present invention provides therapeutic compositions useful for practicing the therapeutic methods described herein. Therapeutic compositions of the present invention contain a physiologically tolerable carrier together with an immunogenic peptide as disclosed herein, or derivatives, fragments or variants thereof or a vector capable of expressing an immunogenic peptide as disclosed herein, or derivatives, fragments or variants thereof as described herein, dissolved or dispersed therein as an active ingredient.

The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution or suspension in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. In addition, if desired, the composition can contain minor amounts of auxiliary substances such as wetting or emulsifying agents, pH buffering agents and the like which enhance the effectiveness of the active ingredient.

Physiologically tolerable carriers (i.e. physiologically acceptable carriers) are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

For topical application, the carrier may be in the form of, for example, and not by way of limitation, an ointment, cream, gel, paste, foam, aerosol, suppository, pad or gelled stick.

The amount of a peptide immunogen as disclosed herein, or derivatives, fragments or variants thereof that will be effective in the treatment of a particular disorder or condition will depend on the nature of the disorder or condition, and can be determined by standard clinical techniques. In addition, assays such as those discussed herein in the Examples may optionally be employed to help identify optimal dosage ranges.

The precise dose to be employed in the formulation will also depend on the route of administration, and the seriousness of the disease or disorder, and should be decided according to the judgment of the practitioner and each subject's circumstances, and is discussed further below.

The route of administration can be any route known to persons skilled in the art, for example but not limited to parenteral, including intravenous and intraarterial administration, intrathecal administration, intraventricular administration, intraparenchymal, intracranial, intracistemal, intrastriatal, and intranigral administration.

In some embodiments, the present invention also contemplates an article of manufacture which is a labeled container for providing an immunogenic peptide as disclosed herein, or derivatives, fragments or variants thereof. An article of manufacture comprises packaging material and a pharmaceutical agent contained within the packaging material.

The pharmaceutical agent in an article of manufacture is any of the compositions of the present invention suitable for providing a peptide immunogen as disclosed herein, or derivatives, fragments or variants thereof and formulated into a pharmaceutically acceptable form as described herein according to the disclosed indications. Thus, the composition can comprise a peptide immunogen as disclosed herein, or derivatives, fragments or variants thereof or a DNA molecule which is capable of expressing the peptide immunogen as disclosed herein, or derivatives, fragments or variants thereof.

The article of manufacture contains an amount of pharmaceutical agent sufficient for use in treating a condition indicated herein, either in unit or multiple dosages. The packaging material comprises a label which indicates the use of the pharmaceutical agent contained therein, e.g., for the treatment of plasma cell dyscrasias, or for other indicated therapeutic or prophylactic uses. The label can further include instructions for use and related information as may be required for marketing. The packaging material can include container(s) for storage of the pharmaceutical agent.

As used herein, the term packaging material refers to a material such as glass, plastic, paper, foil, and the like capable of holding within fixed means a pharmaceutical agent. Thus, for example, the packaging material can be plastic or glass vials, laminated envelopes and the like containers used to contain a pharmaceutical composition including the pharmaceutical agent. In preferred embodiments, the packaging material includes a label that is a tangible expression describing the contents of the article of manufacture and the use of the pharmaceutical agent contained therein.

### X. Treatment Regimes

Effective doses of the compositions of the present invention, for the treatment of the above described clinical symptoms, for example plasma cell malignancies, for example AL amyloidosis or multiple myeloma vary depending upon many different factors, including means of administration, target site, physiological state of the subject, whether the subject is human or an animal, other medications administered, and whether treatment is prophylactic or therapeutic.

The dosage and frequency of administration can vary depending on whether the treatment is prophylactic or therapeutic. In prophylactic applications, a relatively low dosage is administered at relatively infrequent intervals over a long period of time. Some subjects continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In some embodiments, the subject is a human, and in alternative embodiments the subject is a non-human mammal. Treatment dosages need to be titrated to optimize safety and efficacy. The amount of immunogenic peptide depends on the immunogenic peptide being administered as well as whether another antigen, for example an adjuvant is also administered, with higher dosages being required in the absence of adjuvant. The amount of an immunogenic peptide for administration sometimes varies from 1 μg-500 μg per subject and more usually from 5-500 μg per administration for human administration. Occasionally, a higher dose of 0.5-5 mg per injection is used. Typically about 10, 20, 50 or 100 μg is used for administration to a human.

The timing of administration can vary significantly from once a day, to once a year, to once a decade. Generally, in accordance with the teachings provided herein, effective dosages can be monitored by obtaining a fluid sample from the subject, generally a blood serum sample, and determining the titer of antibody developed against the immunogenic peptide, using methods well known in the art and readily adaptable to the specific antigen to be measured. Ideally, a sample is taken prior to initial dosing; subsequent samples are taken and titered after each immunization. Generally, a dose or dosing schedule which provides a detectable titer at least four times greater than control or “background” levels at a serum dilution of 1:100 is desirable, where background is defined relative to a control serum or relative to a plate background in ELISA assays. Titers of at least 1:1000 or 1:5000 are preferred in accordance with the present invention.

On any given day that a dosage of peptide immunogen of the present invention is given, the dosage is greater than about 1 μg/subject and usually greater than 10 μg/subject if adjuvant is also administered, and greater than 10 μg/subject and usually greater than 100 μg/subject in the absence of adjuvant. Doses for individual immunogenic peptide selected in accordance with the present invention, are determined according to standard dosing and titering methods, taken in conjunction with the teachings provided herein. A typical regimen consists of an immunization followed by booster injections at time intervals, such as 6 week intervals. Another regimen consists of an immunization followed by booster injections 1, 2 and 12 months later. Another regimen entails an injection every two months for life. Alternatively, booster injections can be on an irregular basis as indicated by monitoring of immune response. A typical regimen consists of an immunization followed by booster injections at 6 weekly intervals. Another regimen consists of an immunization followed by booster injections 1, 2 and 12 months later. Another regimen entails an injection every two months for life. Alternatively, booster injections can be on an irregular basis as indicated by monitoring of immune response. For passive immunization with an antibody, for example against the peptide immunogen the dosage ranges from about 0.0001 to 100 mg/kg, and more usually 0.01 to 5 mg/kg of the host body weight. Doses for nucleic acids encoding immunogens range from about 10 ng to 1 g, 100 ng to 100 mg, 1 μg to 10 mg, or 30-300 μg DNA per subject. Doses for infectious viral vectors vary from 10-10⁹, or more, virions per dose.

For passive immunization with an antibody, the dosage ranges from about 0.0001 to 100 mg/kg, and more usually 0.01 to 5 mg/kg, of the host body weight. For example dosages can be 1 mg/kg body weight or 10 mg/kg body weight. An exemplary treatment regime entails administration once per every two weeks or once a month or once every 3 to 6 months. In some methods, two or more monoclonal antibodies with different binding specificities are administered simultaneously, in which case the dosage of each antibody administered falls within the ranges indicated. A immunogenic peptide can be administered on multiple occasions. Intervals between single dosages can be weekly, monthly or yearly. Intervals can also be irregular as indicated by measuring blood levels of the titer of antibody developed against the peptide immunogen. Alternatively, the immunogenic peptide can be administered as a sustained release formulation, in which case less frequent administration is required. In embodiments involving antibodies or peptide immunogens, the dosage and frequency vary depending on the half-life of the immunogenic peptide or antibody in the subject. In general, human antibodies show the longest half life, followed by humanized antibodies, chimeric antibodies, and nonhuman antibodies.

The pharmaceutical compositions comprising the immunogenic peptides of the present invention for inducing an immune response can be administered by parenteral, topical, intravenous, oral, subcutaneous, intraperitoneal, intranasal or intramuscular means for prophylactic and/or therapeutic treatment. Typical routes of administration of an immunogenic peptide are intramuscular (i.m.), intravenous (i.v.) or subcutaneous (s.c.), although other routes can be equally effective. Intramuscular injection is most typically performed in the arm or leg muscles. In some methods, the immunogenic peptides or other pharmaceutical compositions are injected directly into a particular tissue, for example a tumor tissue where the immunoglobulin producing cell is located. Such administration is termed intratumoral administration. In some methods, particular pharmaceutical compositions comprising the immunogenic peptides for the treatment of amyloidogenic diseases of the brain are administered directly to the head or brain via injection directly into the cranium. In some methods, antibodies are administered as a sustained release composition or device, such as a Medipad™ device.

Immunogenic peptides of the invention can optionally be administered in combination with other agents that are at least partly effective in treatment of plasma cell malignancies, for example AL amyloidosis and/or multiple myeloma. The immunogenic peptides of the present invention can also be administered in conjunction with other agents that increase passage of the immunogenic peptides of the present invention across the blood-brain barrier. Further, therapeutic cocktails comprising immunogens designed to provoke an immune response against more than one immunoglobulin or protein secreted by plasma cells are also contemplated by the present invention, as are a combination of an immunogenic peptides of the present invention, for example, but not limited to an immunogenic peptide substantially similar to a region of the an immunoglobulin with a lambda 6 light chain and an immunogenic peptide substantially similar to a region of the a different lambda light chain, for example immunoglobulin light chain 2, or an immunogenic peptide substantially similar to a region of Blimp-1.

In some embodiments, the immunogenic peptides of the present invention are optionally administered in combination with an adjuvant. A variety of adjuvants can be used in combination with the immunogenic peptides of the present invention, for example lambda 6(2-10) peptide, to elicit an immune response. In some embodiments the adjuvants augment the intrinsic response to an peptide immunogen of the present invention without causing conformational changes in the immunogen that affect the qualitative form of the response. In some embodiments the adjuvants is Freud's Complete Adjuvant. In alternative embodiments, the adjuvant is, for example but not limited to alum, 3 De-O-acylated monophosphoryl lipid A (MPL™) (see GB 2220211). QS21 is a triterpene glycoside or saponin isolated from the bark of the Quillaja Saponaria Molina tree found in South America (see Kensil et al., in Vaccine Design: The Subunit and Ajuvant Approach (eds. Powell & Newman, Plenum Press, N.Y., 1995); U.S. Pat. No. 5,057,540). Other adjuvants useful are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG (Bioworld Today, Nov. 15, 1998). Alternatively, the peptide of the present invention can be coupled to an adjuvant. For example, a lipopeptide version of an immunogenic peptide can be prepared by coupling palmitic acid or other lipids directly to the N-terminus of the immunogenic peptide as described for hepatitis B antigen vaccination (Livingston, J. Immunol. 159, 1383-1392 (1997)). However, such coupling should not substantially change the conformation of the immunogenic peptide as to affect the nature of the immune response thereto. Adjuvants can be administered as a component of a therapeutic composition with an active agent or can be administered separately, before, concurrently with, or after administration of the therapeutic agent.

In an alternative embodiment, the class of adjuvants is aluminum salts (alum), such as aluminum hydroxide, aluminum phosphate, aluminum sulfate. Such adjuvants can be used with or without other specific immunostimulating agents such as MPL or 3-DMP, QS-21, polymeric or monomeric amino acids such as polyglutamic acid or polylysine. Another class of adjuvants is oil-in-water emulsion formulations. Such adjuvants can be used with or without other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1-2dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™, or other bacterial cell wall components. Oil-in-water emulsions include (a) MF59 (WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton Mass.), (b) SAF, containing 10% Squalene, 0.4% Tween 80, 5% pluronic-blocked polymer L121, and thr-MDP, either microfluidized into a submicron emulsion or vortexed to generate a larger particle size emulsion, and (c) Ribi™ adjuvant system (RAS), (Ribi Immunochem, Hamilton, Mont.) containing 2% squalene, 0.2% Tween 80, and one or more bacterial cell wall components from the group consisting of monophosphoryl lipid A, trehalose dimycolate (TDM), and cell wall skeleton (CWS), preferably MPL+CWS (DetoX™). Another class of preferred adjuvants is saponin adjuvants, such as Stimulon™ (QS-21; Aquila, Framingham, Mass.) or particles generated therefrom such as ISCOMs (immunostimulating complexes) and ISCOMATRIX. Other adjuvants include Incomplete Freund's Adjuvant (IFA), cytokines, such as interleukins (IL-1, IL-2, and IL-12), macrophage colony stimulating factor (M-CSF), and tumor necrosis factor (TNF). Such adjuvants are generally available from commercial sources.

An adjuvant can be administered with an immunogenic peptide as a single composition, or can be administered before, concurrent with or after administration of the immunogenic peptide. Immunogenic peptides and adjuvants can be packaged and supplied in the same vial or can be packaged in separate vials and mixed before use. Immunogen and adjuvant are typically packaged with a label indicating the intended therapeutic application. If the immunogenic peptide and adjuvant are packaged separately, the packaging typically includes instructions for mixing before use. The choice of an adjuvant and/or carrier depends on such factors as the stability of the formulation containing the adjuvant, the route of administration, the dosing schedule, and the efficacy of the adjuvant for the species being vaccinated. In humans, a preferred pharmaceutically acceptable adjuvant is one that has been approved for human administration by pertinent regulatory bodies. Examples of such preferred adjuvants for humans include alum, MPL and QS-21. Optionally, two or more different adjuvants can be used simultaneously. Preferred combinations include alum with MPL, alum with QS-21, MPL with QS-21, and alum, QS-21 and MPL together. Also, Incomplete Freund's adjuvant can be used (Chang et al., Advanced Drug Delivery Reviews 32, 173-186 (1998)), optionally in combination with any of alum, QS-21, and MPL and all combinations thereof.

In some embodiments, the immunogenic peptides of the invention are often administered as pharmaceutical compositions comprising an active therapeutic agent, i.e., and a variety of other pharmaceutically acceptable components. See Remington's Pharmaceutical Science (15th ed., Mack Publishing Company, Easton, Pa., 1980). The form of administration depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, non-therapeutic, non-immunogenic stabilizers and the like. However, some reagents suitable for administration to animals may not necessarily be used in compositions for human use.

Pharmaceutical compositions can also optionally comprise include large, slowly metabolized macromolecules such as proteins, polysaccharides, polylactic acids, polyglycolic acids and copolymers (such as latex functionalized sepharose, agarose, cellulose, and the like), polymeric amino acids, amino acid copolymers, and lipid aggregates (such as oil droplets or liposomes). Additionally, these carriers can function as immunostimulating agents (i.e., adjuvants), or targeting carries to target the immunogenic peptide to specific target cells or target organs, for example the bone marrow as a target organ or plasma cells as target cells.

For parenteral administration, the immunogenic peptide of the present invention can be administered as injectable dosages of a solution or suspension of the substance in a physiologically acceptable diluent with a pharmaceutical carrier which can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249, 1527 (1990) and Hanes, Advanced Drug Delivery Reviews 28, 97-119 (1997). The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications. For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein.

Alternatively, transdermal delivery can be achieved using a skin path or using transferosomes (Paul et al., Eur. J. Immunol. 25, 3521-24 (1995); Cevc et al., Biochem. Biophys. Acta 1368, 201-15 (1998)).

A. Nucleic acid encoding the immunogenic peptides. The immunogenic peptides of the present invention used to elicit an immune response against plasma cells or B cells can also be induced by administration of nucleic acids encoding selected immunogenic peptides, or antibodies or regions thereof used for passive immunization. Such nucleic acids can be DNA or RNA. A nucleic acid segment encoding an immunogenic peptide of the present invention is typically linked or operatively linked to regulatory elements, such as a promoter and enhancer that allows for expression of the DNA segment in the intended target cells of a subject. For expression in blood cells, as is desirable for induction of an immune response, promoter and enhancer elements from light or heavy chain immunoglobulin genes or the CMV major intermediate early promoter and enhancer are suitable to direct expression. The linked regulatory elements and coding sequences are often cloned into a vector. For administration of double-chain antibodies, the two chains can be cloned in the same or separate vectors.

Without limitation and as well-known to those skilled in the art, the invention encompasses the nucleic acid sequences that encode the immunogenic peptides of the present invention, for example but not limited nucleic acid sequences which encode the peptides of SEQ ID NO: 1 to 8, or derivatives, analogues, variants or homologues thereof. The nucleic acid sequences can further include vector DNA, such that the coding region can be introduced into a host cell. Also encompassed are those complementary to DNA, mRNA, and tRNA encoding the immunogenic peptides of the present invention. With respect to the lambda 6 light chain proteins of this invention, the degeneracy of the genetic code is contemplated such that the position 38 and analogous residues can be specified by more than one codon.

A number of viral vector systems are available including retroviral systems (see, e.g., Lawrie and Tumin, Cur. Opin. Genet. Develop. 3, 102-109, 1993); adenoviral vectors (see, e.g., Bett et al., J. Virol. 67, 5911, 1993); adeno-associated virus vectors (see, e.g., Zhou et al., J. Exp. Med. 179, 1867, 1994), viral vectors from the pox family including vaccinia virus and the avian pox viruses, viral vectors from the alpha virus genus such as those substantially similar to a region of the Sindbis and Semliki Forest Viruses (see, e.g., Dubensky et al., J. Virol. 70, 508-519, 1996), Venezuelan equine encephalitis virus (see U.S. Pat. No. 5,643,576) and rhabdoviruses, such as vesicular stomatitis virus (see WO 96/34625) and papillomaviruses (Ohe et al., Human Gene Therapy 6, 325-333, 1995); Woo et al., WO 94/12629 and Xiao Brandsma, Nucleic Acids. Res. 24, 2630-2622, 1996).

These vectors can be viral vectors such as adenovirus, adeno-associated virus, pox virus such as an orthopox (vaccinia and attenuated vaccinia), avipox, lentivirus, murine moloney leukemia virus, etc. Alternatively, plasmid expression vectors can be used.

Viral vector systems which can be utilized in the present invention include, but are not limited to, (a) adenovirus vectors; (b) retrovirus vectors; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. In a preferred embodiment, the vector is an adenovirus. Replication-defective viruses can also be advantageous. The vector may or may not be incorporated into the cells genome. The constructs may include viral sequences for transfection, if desired. Alternatively, the construct may be incorporated into vectors capable of episomal replication, e.g EPV and EBV vectors.

Constructs for the recombinant expression of an peptide immunogen as disclosed herein, such as SEQ ID NO: 1 to 8 will generally require regulatory elements, e.g., promoters, enhancers, etc., to ensure the expression of the construct in target cells. Other specifics for vectors and constructs are described in further detail below.

As used herein, a “promoter” or “promoter region” or “promoter element” used interchangeably herein, refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated.

The term “regulatory sequences” is used interchangeably with “regulatory elements” herein refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked. Regulatory sequence often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation.

Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

The term “operatively linked” as used herein refers to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined. In some embodiments, it can be advantageous to direct expression of a peptide immunogen in a tissue- or cell-specific manner.

In a specific embodiment, viral vectors that contain nucleic acid sequences encoding a peptide immunogen or fragments or derivatives or variants thereof are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding a peptide immunogen or fragments or variants thereof are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

The production of a recombinant retroviral vector carrying a gene of interest is typically achieved in two stages. First, sequence encoding a peptide immunogen or fragments or derivatives or variants thereof can be inserted into a retroviral vector which contains the sequences necessary for the efficient expression of the metabolic regulators (including promoter and/or enhancer elements which can be provided by the viral long terminal repeats (LTRs) or by an internal promoter/enhancer and relevant splicing signals), sequences required for the efficient packaging of the viral RNA into infectious virions (e.g., a packaging signal (Psi), a tRNA primer binding site (—PBS), a 3[prime] regulatory sequence required for reverse transcription (+PBS)), and a viral LTRs). The LTRs contain sequences required for the association of viral genomic RNA, reverse transcriptase and integrase functions, and sequences involved in directing the expression of the genomic RNA to be packaged in viral particles.

Following the construction of the recombinant retroviral vector, the vector DNA is introduced into a packaging cell line. Packaging cell lines provide viral proteins required in trans for the packaging of viral genomic RNA into viral particles having the desired host range (e.g., the viral-encoded core (gag), polymerase (pol) and envelope (env) proteins). The host range is controlled, in part, by the type of envelope gene product expressed on the surface of the viral particle. Packaging cell lines can express ecotrophic, amphotropic or xenotropic envelope gene products. Alternatively, the packaging cell line can lack sequences encoding a viral envelope (env) protein. In this case, the packaging cell line can package the viral genome into particles which lack a membrane-associated protein (e.g., an env protein). To produce viral particles containing a membrane-associated protein which permits entry of the virus into a cell, the packaging cell line containing the retroviral sequences can be transfected with sequences encoding a membrane-associated protein (e.g., the G protein of vesicular stomatitis virus (VSV)). The transfected packaging cell can then produce viral particles which contain the membrane-associated protein expressed by the transfected packaging cell line; these viral particles which contain viral genomic RNA derived from one virus encapsidated by the envelope proteins of another virus are said to be pseudotyped virus particles.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Another preferred viral vector is a pox virus such as a vaccinia virus, for example an attenuated vaccinia such as Modified Virus Ankara (MVA) or NYVAC, an avipox such as fowl pox or canary pox. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In another embodiment, lentiviral vectors are used, such as the HIV based vectors described in U.S. Pat. Nos. 6,143,520; 5,665,557; and 5,981,276, which are herein incorporated by reference. Use of Adeno-associated virus (AAV) vectors is also contemplated (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient. Such cells, can be for example but are not limited to, cells in the blood and plasma, as well as bone marrow cells.

U.S. Pat. No. 5,676,954 (which is herein incorporated by reference) reports on the injection of genetic material, complexed with cationic liposome carriers, into mice. U.S. Pat. Nos. 4,897,355, 4,946,787, 5,049,386, 5,459,127, 5,589,466, 5,693,622, 5,580,859, 5,703,055, and international publication NO: WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication NO: WO 94/9469 (which are herein incorporated by reference) provide methods for delivering DNA-cationic lipid complexes to mammals. Such cationic lipid complexes or nanoparticles can also be used to deliver protein.

A gene or nucleic acid sequence can be introduced into a target cell by any suitable method. For example, an a nucleoic acid sequence encoding a peptide immunogen or fragments or derivatives or variants thereof can be introduced into a cell by transfection (e.g., calcium phosphate or DEAE-dextran mediated transfection), lipofection, electroporation, microinjection (e.g., by direct injection of naked DNA), biolistics, infection with a viral vector containing a muscle related transgene, cell fusion, chromosome-mediated gene transfer, microcell-mediated gene transfer, nuclear transfer, and the like. A nucleic acid encoding an peptide immunogen or fragments or derivatives or variants thereof are used can be introduced into cells by electroporation (see, e.g., Wong and Neumann, Biochem. Biophys. Res. Commun. 107:584-87 (1982)) and biolistics (e.g., a gene gun; Johnston and Tang, Methods Cell Biol. 43 Pt A:353-65 (1994); Fynan et al., Proc. Natl. Acad. Sci. USA 90:11478-82 (1993)).

In certain embodiments, a gene or nucleic acid sequence encoding a peptide immunogen or fragments or derivatives or variants thereof can be introduced into target cells by transfection or lipofection. Suitable agents for transfection or lipofection include, for example, calcium phosphate, DEAE dextran, lipofectin, lipfectamine, DIMRIE C, Superfect, and Effectin (Qiagen), unifectin, maxifectin, DOTMA, DOGS (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., J. Pharm. 183:195-207 (1999)).

Methods known in the art for the therapeutic delivery of agents such as proteins and/or nucleic acids can be used for the delivery of a polypeptide or nucleic acid encoding a peptide immunogen or fragments or derivatives or variants thereof are used treating and/or preventing plasma cell dyscrasias in a subject, e.g., cellular transfection, gene therapy, direct administration with a delivery vehicle or pharmaceutically acceptable carrier, indirect delivery by providing recombinant cells comprising a nucleic acid encoding a targeting fusion polypeptide of the invention.

Various delivery systems are known and can be used to directly administer therapeutic peptide immunogens as disclosed herein, or fragments or derivatives or variants thereof, e.g., encapsulation in liposomes, microparticles, microcapsules, recombinant cells capable of expressing the compound, and receptor-mediated endocytosis (see, e.g., Wu and Wu, 1987, J. Biol. Chem. 262:4429-4432). Methods of introduction can be enteral or parenteral and include but are not limited to intradermal, intramuscular, intraperitoneal, intravenous, subcutaneous, pulmonary, intranasal, intraocular, epidural, and oral routes. The agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, the implant being of a porous, non-porous, or gelatinous material, including membranes, such as sialastic membranes, fibers, or commercial skin substitutes.

In another embodiment, peptide immunogens or fragments or derivatives or variants thereof as disclosed herein can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105).

Thus, a wide variety of gene transfer/gene therapy vectors and constructs are known in the art. These vectors are readily adapted for use in the methods of the present invention. By the appropriate manipulation using recombinant DNA/molecular biology techniques to insert an operatively linked nucleic acid sequence encoding a peptide immunogen as disclosed herein, or fragments or derivatives or variants thereof, into the selected expression/delivery vector, many equivalent vectors for the practice of the methods described herein can be generated.

It will be appreciated by those of skill that cloned genes readily can be manipulated to alter the amino acid sequence of a peptide immunogen or fragments or derivatives or variants thereof. The cloned nucleic acid sequence for a peptide immunogen as disclosed herein can be manipulated by a variety of well known techniques for in vitro mutagenesis, among others, to produce variants of the naturally occurring human protein, herein referred to as variants or muteins or mutants of the peptide immunogen, which may be used in accordance with the methods and compositions described herein.

The variation in primary structure of a peptide immunogen as disclosed herein are useful in the present invention, for instance, may include deletions, additions and substitutions. The substitutions may be conservative or non-conservative. The differences between the natural protein (native) and the variant of a peptide immunogen generally conserve desired properties, mitigate or eliminate undesired properties and add desired or new properties.

In some embodiments, the peptide immunogen as disclosed herein, or variants or fragments or derivatives thereof can also be a fusion polypeptide, fused, for example, to a polypeptide that targets the product to a desired location, or, for example, a tag that facilitates its purification, if so desired. Fusion to a polypeptide sequence that increases the stability of the peptide immunogen is also contemplated, without substantially interfering with its ability to induce a CTL response in vivo or in vitro is desired For example, fusion to a serum protein, e.g., serum albumin, can increase the circulating half-life of a peptide immunogen. Tags and fusion partners can be designed to be cleavable, if so desired. Another modification specifically contemplated is attachment, e.g., covalent attachment, to a polymer. In one aspect, polymers such as polyethylene glycol (PEG) or methoxypolyethylene glycol (mPEG) can increase the in vivo half-life of proteins to which they are conjugated. Methods of PEGylation of polypeptide agents are well known to those skilled in the art, as are considerations of, for example, how large a PEG polymer to use.

In another aspect, biodegradable or absorbable polymers can provide extended, often localized, release of polypeptide agents. The potential benefits of an increased half-life or extended release for a therapeutic agent are clear. A potential benefit of localized release is the ability to achieve much higher localized dosages or concentrations, for greater lengths of time, relative to broader systemic administration, with the potential to also avoid possible undesirable side effects that may occur with systemic administration.

Bioabsorbable polymeric matrix suitable for delivery of peptide immunogens as disclosed herein, or variants or fragments or derivatives thereof can be selected from a variety of synthetic bioabsorbable polymers, which are described extensively in the literature. Such synthetic bioabsorbable, biocompatible polymers, which may release proteins over several weeks or months can include, for example, poly-α-hydroxy acids (e.g. polylactides, polyglycolides and their copolymers), polyanhydrides, polyorthoesters, segmented block copolymers of polyethylene glycol and polybutylene terephtalate (Polyactive™), tyrosine derivative polymers or poly(ester-amides). Suitable bioabsorbable polymers to be used in manufacturing of drug delivery materials and implants are discussed e.g. in U.S. Pat. Nos. 4,968,317, 5,618,563, among others, and in “Biomedical Polymers” edited by S. W. Shalaby, Carl Hanser Verlag, Munich, Vienna, New York, 1994 and in many references cited in the above publications. The particular bioabsorbable polymer that should be selected will depend upon the particular patient that is being treated.

DNA encoding immunogenic peptide of the present invention, or a vector containing the same, can be packaged into liposomes. Suitable lipids and related analogs are described by U.S. Pat. Nos. 5,208,036, 5,264,618, 5,279,833 and 5,283,185. Vectors and DNA encoding an immunogen can also be adsorbed to or associated with particulate carriers, examples of which include polymethyl methacrylate polymers and polylactides and poly(lactide-co-glycolides), see, e.g., McGee et al., J. Micro Encap. (1996).

Gene therapy vectors or naked DNA can be delivered in vivo by administration to an individual patient, typically by systemic administration (e.g., intravenous, intraperitoneal, intranasal, gastric, intradermal, intramuscular, subdermal, or intracranial infusion) or topical application (see e.g., U.S. Pat. No. 5,399,346). Such vectors can further include facilitating agents such as bupivacaine (U.S. Pat. No. 5,593,970). DNA can also be administered using a gene gun. See Xiao Brandsma, supra. The DNA encoding an immunogen is precipitated onto the surface of microscopic metal beads. The microprojectiles are accelerated with a shock wave or expanding helium gas, and penetrate tissues to a depth of several cell layers. For example, The Accel™ Gene Delivery Device manufactured by Agracetus, Inc., (Middleton, Wis.) is suitable. Alternatively, naked DNA can pass through skin into the blood stream simply by spotting the DNA onto skin with chemical or mechanical irritation (see WO 95/05853).

In a further variation, vectors encoding immunogenic peptides or the present invention can be delivered to cells ex vivo, such as cells explanted from a subject (e.g., lymphocytes, bone marrow aspirates, tissue biopsy) or universal donor hematopoietic stem cells, followed by reimplantation of the cells into a subject, usually after selection for cells which have incorporated the vector, and expression of the nucleic acid encoding the immunogenic peptide of the present invention.

### XI. Methods of Diagnosis

The methods of the present invention also are useful for monitoring a course of treatment being administered to a subject. The methods can be used to monitor both therapeutic treatment on symptomatic subject and prophylactic treatment on asymptomatic subject.

In some embodiments, such methods entail determining a baseline value of an immune response in a subject before administering a dosage of agent, and comparing this with a value for the immune response after treatment. A significant increase (i.e., greater than the typical margin of experimental error in repeat measurements of the sane sample, expressed as one standard deviation from the mean of such measurements) in value of the immune response signals a positive treatment outcome (i.e., that administration of the agent has achieved or augmented an immune response). If the value for immune response does not change significantly, or decreases, a negative treatment outcome is indicated. In general, subject undergoing an initial course of treatment with an agent are expected to show an increase in immune response with successive dosages, which eventually reaches the plateau. Administration of agent is generally continued while the immune response is increasing. Attainment of the plateau is an indicator that the administered of treatment can be discontinued or reduced in dosage or frequency.

In other methods, a control value (i.e., a mean and standard deviation) of immune response is determined for a control population. Typically the subjects in the control population have not received prior treatment. Measured values of immune response in a patient after administering a therapeutic agent are then compared with the control value. A significant increase relative to the control value (e.g., greater than one standard deviation from the mean) signals a positive treatment outcome. A lack of significant increase or a decrease signals a negative treatment outcome. Administration of agent is generally continued while the immune response is increasing relative to the control value. As before, attainment of a plateau relative to control values in an indicator that the administration of treatment can be discontinued or reduced in dosage or frequency.

In other methods, a control value of immune response (e.g., a mean and one standard deviation) is determined from a control population of subjects who have undergone treatment with a therapeutic agent and whose immune responses have plateaued in response to treatment. Measured values of immune response in a patient are compared with the control value. If the measured level in a patient is not significantly different (e.g., more than one standard deviation) from the control value, treatment can be discontinued. If the level in a subject is significantly below the control value, continued administration of agent is warranted. If the levels in the patient persist below the control value, then a change in treatment regime, for example, use of a different adjuvant may be indicated.

In other methods, a patient who is not presently receiving treatment but has undergone a previous course of treatment is monitored for immune response to determine whether a resumption of treatment is required. The measured value of immune response in the patient can be compared with a value of immune response previously achieved in the patient after a previous course of treatment. A significant decrease relative to the previous measurement (i.e., greater than a typical margin of error in repeat measurements of the same value) is an indication that treatment can be resumed. Alternatively, the value measured in subject can be compared with a control value (mean plus standard deviation) determined in population of subjects after undergoing a course of treatment. Alternatively, the measured value in a subject can be compared with a control value in populations of prophylactically treated subjects who remain free of symptoms of disease, or populations of therapeutically treated subjects who show amelioration of disease characteristics. In all of these cases, a significant decrease relative to the control level (i.e., more than a standard deviation) is an indicator that treatment should be resumed in a subject.

In some embodiments, the tissue sample for analysis is typically blood, plasma, serum, urine, mucus or cerebral spinal fluid from the subject. The sample is analyzed for an immune response to any forms of the immunoglobulin light chain lambda, for example a response to immunoglobulins comprising lambda 6. The immune response can be determined from the presence of, e.g., antibodies or T-cells that specifically bind to the immunogenic peptides of the present invention. ELISA methods of detecting antibodies specific to the immunogenic peptides are commonly known in the art and are encompassed for use in the present invention. Methods of detecting reactive T-cells have been described herein, and are useful in the methods of the present invention.

### XII. Methods to Screen Immunogenic Peptides of the Present Invention

In another aspect of the invention relates to methods to design immunogenic peptides for the treatment of plasma cell disorders and other malignancies, for example cancers and tumor malignancies. In some embodiments the invention provides methods to design immunogenic peptides substantially similar to a region of the proteins which are expressed or overexpressed by malignant cells, for example plasma cells and/or tumor cells. Accordingly, the present invention provides methods to design peptide immunogens for treatment of such disorders such as plasma cell disorders and malignancies, for example tumor malignancies. In such an embodiment, the method of the present invention uses a screen to identify overexpressed proteins from the plasma cell or malignant cell, and then use of algorithms to assess the stability to form complexes with MHC Class I molecules. For example, in such an embodiment the method of the present invention comprises; (i) a screen to identify proteins expressed only in, or a higher level in the plasma cells or malignant cells from the subject afflicted with a malignancy as compared with a normal subject, (ii) assessing the immunogenic peptides that are substantially similar to a region of the identified protein for stability to form complex with MHC Class I. The method to design immunogenic peptides may optionally comprise of assessing the ability of immunogenic peptides predicated to form stable complexes with MHC Class I to elicit a CTL response in vitro or in vivo. Suitable immunogenic peptides that are predicted to form stable complexes with MHC Class I molecules are then assessed for their ability to induce a CTL as discussed herein in the examples.

The immunogenic peptide of the present invention is any peptide substantially similar to a region of the a protein expressed by plasma cells or malignant cell, for example cancer cell or amyloidogenic cell, that has specific binding to MHC Class I, as demonstrated in the Examples. As used herein, the term “specific binding” refers to the ability of the immunogenic peptide to form a stable complex with the MHC Class I molecules. The binding affinities and prediction of the stability to complex with MHC Class I molecules can be predicted by algorithms that are known by persons of ordinary skill in the art, for example but not limited to theses used in the Examples, such as NIH-BIMAS algorithm, SYFPEITHI algorithm and Boston University ZLAB algorithm as well as others that are available on the internet or web, as discussed in the Examples. In some embodiments, a predicted binding or stability between the peptide immunogen of the present invention and the MHC Class I molecules using such algorithms means that the immunogenic peptide and MHC Class I molecule will form stable complexes in vitro and in vivo. In some embodiments, the binding affinities will be at least 10³, 10⁴ and 10⁵, 10⁶, 10⁷, 10⁸, 10⁹ M⁻¹, or 10¹⁰ M⁻¹. Suitable immunogenic peptides that are predicted to form stable complexes with MHC Class I molecules are then assessed for their ability to induce a CTL in vitro and in vivo, as discussed herein in the examples by injecting the selected immunogenic peptide into an animal model, for example HHD mice, and monitoring ability to elicit a CTL response, as well as the ability of the CLT to kill cells comprising the immunogenic peptide of the present invention or comprising a protein with a region substantially similar to the immunogenic peptide that was used to immunize the HHD mice. If such an immunogenic peptide elicits such a CTL response and is capable of killing targeted cell, it can be used in part of a pharmaceutical composition to treat plasma cell disorders and other malignancies, for example cancers and amyloidogenic diseases, as well as plasma cell dyscrasias according to the methods of the present invention.

### EXAMPLES

The examples presented herein relate to the design and use of immunogenic peptides for the treatment of subjects with plasma cell disorders, for example plasma cell dyscrasias and other malignancies. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Methods

Mice: HHD mice on C67BL6/J background, transgenic for human MHC Class I (α1 and α2 domains from HLAA*0201, α3 domain from H2-K^{d}) and knockout of H2-D^{b} (mouse MHC Class I).

Peptides: Peptides at >85% purity were obtained from New England Peptide and resuspended in PBS at 4 mg/ml.

Immunizations: 200 μg peptide plus 200 μg HBVc “helper” peptide (to elicit CD4+ T cell help), emulsified in complete Freud's Adjuvant (IFA) is used to immunize the HHD mice subcutaneously in the lower abdominal area.

Mouse T Cell Culture: Spleens were harvested from the mice and dissociated using sterile frosted glass slides to yield single-cell suspension. Spleenocytes are plated at approximately 1×10⁶ cells/ml and 10% FBS supplemented with L-glutamine, non-essential amino acids and β2-mercaptoethanol. Ex vivo cells were stimulated with 10 μg/ml peptide and fed every 2-3 days with medium and 2 ng/ml rIL-2 for 7-10 days.

Cell Lines: Human cell lines which naturally express the HLAA*0201 allele of MHC Class I are grown in RPMI 1640 and 10% FBS supplemented with L-glutamine. Cell lines include the JY and U66 lymphobastoma cell lines of B cell origin and the T2 cell line of T cell origin.

Killing Assay: Target cell lines are loaded with radioactive sodium chromate (⁵¹Cr) and pulsed with 20 μg/ml peptide or as a negative control peptide, flu matrix protein MP-58. Spleen cells are plated with targets at various effector target ratios and incubated at 37° C., 5% CO₂ overnight. Supernatants are harvested from each well and assayed for radioactive chromium indicating release from lysed cells. % specific lysis was determined by subtracting the control minimum release from experimental wells and dividing by maximum-minimum release.

Fluorescent Analysis: At the time of the killing assay, a sample of each splenocyte population is stained for flow cytometic analysis (FACS) using fluorescently-labeled anti-CD8 monoclonal antibodies and pentamers. The pentamer is comprised of 5 copies of an HLAA*0201 molecule, each covalently linked to the λ6 peptide. The peptide/MHC Class I complex is linked to fluorochromes, such that when a T cell specific fir the peptide/MHC complex binds the pentamer it is detectable by flow cytochemistry.

Statistics: Statistical analysis was performed with Statview. Data from killing assays of a minimum of three mice are presented as means ± standard errors (SE). ANOVA was used to determine the statistical significance compared to the negative control peptide.

### Example 1

Design of immunogenic peptides. The inventors have discovered a method to exploit the plasticity of the immune system to develop a less toxic, more directed approach to plasma cell dyscrasias. Specifically, the inventors have discovered, using specifically designed peptides, a method to induce cytolytic T cells (CTL) capable of killing immunoglobulin light chain-producing B or plasma cells. Accordingly, the inventors have discovered a method for use of peptides as a vaccine for AL amyloidosis or myleoma. By sequencing of DNA from bone marrow aspirates from 264 AL amyloid subjects, the inventors discovered that the lambda 6 immunoglobulin light chain subtype was significant over-representation in AL amyloid subjects (Table 1), suggesting an important role for this type of light chain in AL amyloidosis. Therefore, the inventors designed peptides to be used as vaccines, where the peptides were substantially similar to a region of this immunoglobulin light chain to target the cells producing the pathogenic light chain.

The inventors assessed each peptide for the ability of the peptide to complex with the major histocomplatibility complex class I (MHC class I), which enabled them to discover peptides capable of eliciting the type of immune response (CD8+ cytotoxic T lymphocytes/CTL). To induce such a CD8+/CTL response, a peptide, typically substantially similar to a region of the target protein, must bind to the major histocompatibility complex class I protein (MHC class I) on the surface of specialized immune cells. A peptide which is associated with the MHC class I on these cells then is presented to the killer T cell arm of the immune system.

TABLE 1
Frequency of Immunglobulin Subtypes in AL amyloidosis.

| | Percent | Percent |
|---|---|---|
| Light | Representation in | Representation in |
| Chain | AL Amyloidosis | Normal B Cell Repertoire* |
| Λ1 | 21.1 | 18.6 |
| λ2 | 16.1 | 17.9 |
| λ3 | 18.8 | 32.0 |
| λ4 | 0.9 | 1.5 |
| λ5 | 0 | 0.3 |
| λ6 | 16.6 | 1.5 |
| λ7 | 0.5 | 0.5 |
| λ8 | 0 | 1.5 |
| λ9 | 0 | 0.5 |
| λ10 | 0.5 | 0.3 |
| κ1 | 21.5 | NT |
| κ2 | 0.5 | NT |
| κ3 | 0.5 | NT |
| κ4 | 3.2 | NT |

CTL responses against light chains highlighted by a * have been generated.
Cross-reactivity against those with a ∞ are suspected.

Potentially immunogenic lambda light chain-derived nanomer peptides were identified in silico. One of these peptides is a 9mer (nine amino acid peptide sequence) called lambda 6(2-10), also referred to herein as “λ6₂₋₁₀” and has the peptide sequence of FMLTQPHSV (SEQ ID NO:1). Another peptide analysed is a heteroclitic (hc) variant of lambda 6(2-10), also referred to herein as “heteroclitic lambda 6(2-10)” or “het λ6₂₋₁₀” and has the peptide sequence of YMLTQPHSV (SEQ ID NO:2), where bold indicates the altered amino acid in heterolytic peptide sequence. Other heteroclitic lambda 6(2-10) peptides were tested (data not shown) which had the peptide sequence of FLLTQPHSV (SEQ ID NO:3) (data not shown). These heteroclitic peptides have altered MHC anchor residues resulting in a more stabilized peptide/MHC compleses, thus enabling more opportunity for T cells to identify peptides presented by the APC in vivo, and this subsequent T cell activation.

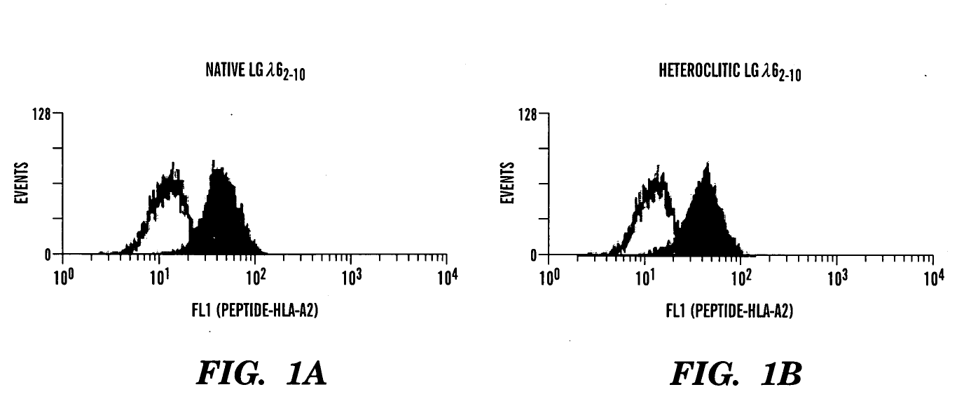

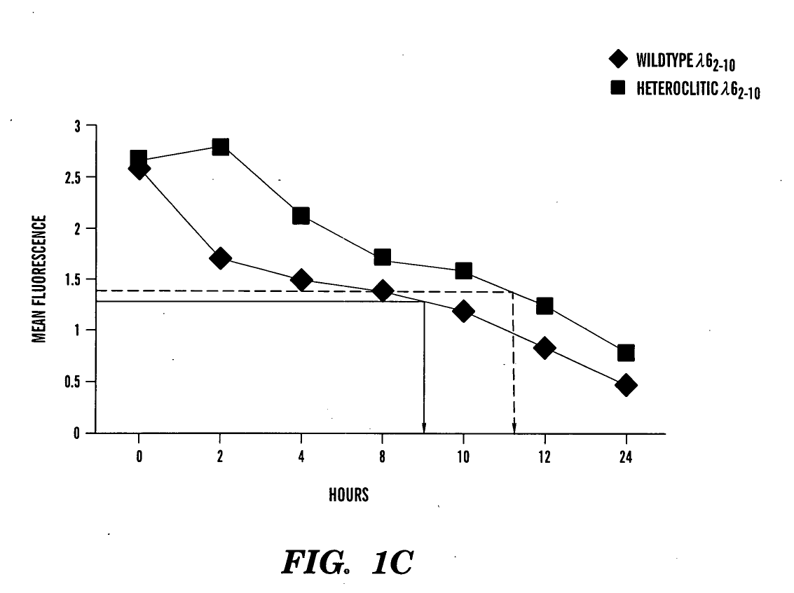

The inventors used three web-based algorithms, which predict peptide-MHC class I binding stability and thereby the relative immunogenicity of any peptide, to identify a conserved lambda 6 light chain framework 1-derived peptide (lambda 6 (2-10) that is predicted to bind to HLA-*A0201 with relatively high affinity (Table 2). HLA-A*0201 was chosen as the human MHC class I allele, since this MHC class I allele is expressed in a large fraction, approximately ˜50%, of the human population. Using cell-based assays, the inventors confirmed the relatively long half-life of HLA-A2 complexes formed with lambda 6 (2-10) peptide (FIG. 1) confirming that the lambda 6(2-10) peptide is a good immunogen.

Table 2 shows Human light chain sequences that were evaluated by the NIH-BIMAS (Bioinformatics and Molecular Analysis Section), SYFPEITHI, and Boston University ZLAB algorithms for predicting peptide-HLA-A2 binding. The NIH-BIMAS algorithm predicted a half-life (minutes) of peptide-HLA-A2 complexes where a higher numbers indicate greater stability. The SYFPEITHI algorithm shows a relative scores where higher scores indicate greater stability (Max 36). The Boston University ZLAB algorithm shows a Log IC50 where lower numbers indicate greater stability. This the inventor discovered that using all three algorithms, a relatively long half-life of HLA-A2 complexes formed with lambda 6 (2-10) peptide, but a longer half-life of HLA-A2 complexes formed with the heteroclitic lambda 6 (2-10) peptide.

TABLE 2
Lambda 6 peptide scores in 3 peptide-MHC Class I binding algorithms.

| | | | HLA-A*0201 Binding Scores | | |
|---|---|---|---|---|---|
| | | | | SYFPEITHI | |
| | | | NIH-BIMAS | Relative | B.U. ZLAB |
| Light Chain | Sequence | Location | (Half-life in mins) | score from 1-36) | (Log IC₅₀) |
| λ6₂₋₁₀ | FMLTQPHSV | FR1 | 846 | 24 | 2.59 |
| het λ6₂₋₁₀ | YMLTQPHSV | FR1 | 1083 | 27 | 0.78 |

The inventors also designed a slightly modified version of the native peptide, wherein 1 amino acid substitution at position 1 of the 9mer peptide of the native peptide is modified (referred to herein as “heteroclitic” peptide). One such heteroclitic peptide the inventors discovered has the peptide sequence of YMLTQPHSV (SEQ ID NO: 2) which binds HLA-A2 with a higher affinity and would therefore likely be a stronger immunogen (see FIG. 1 and Table 2).

### Example 2

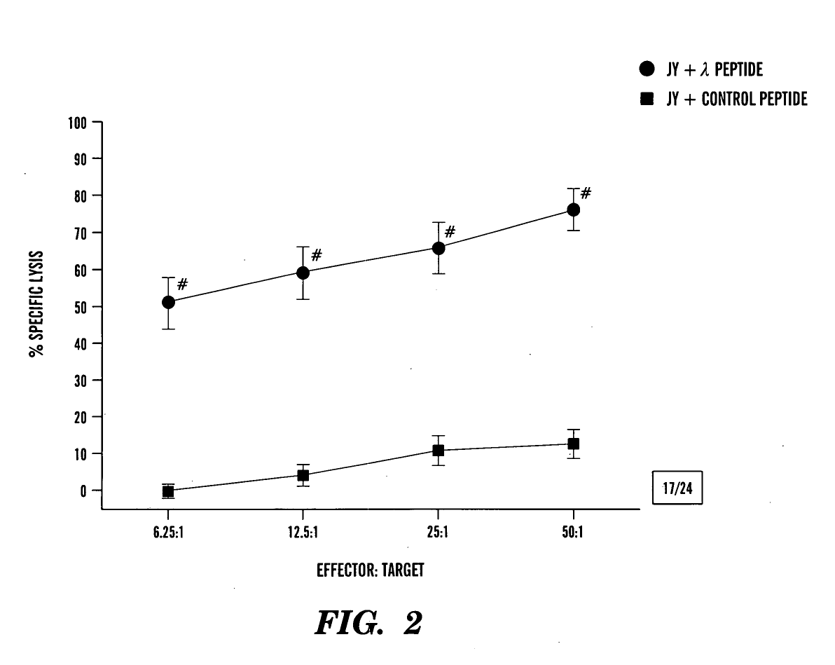

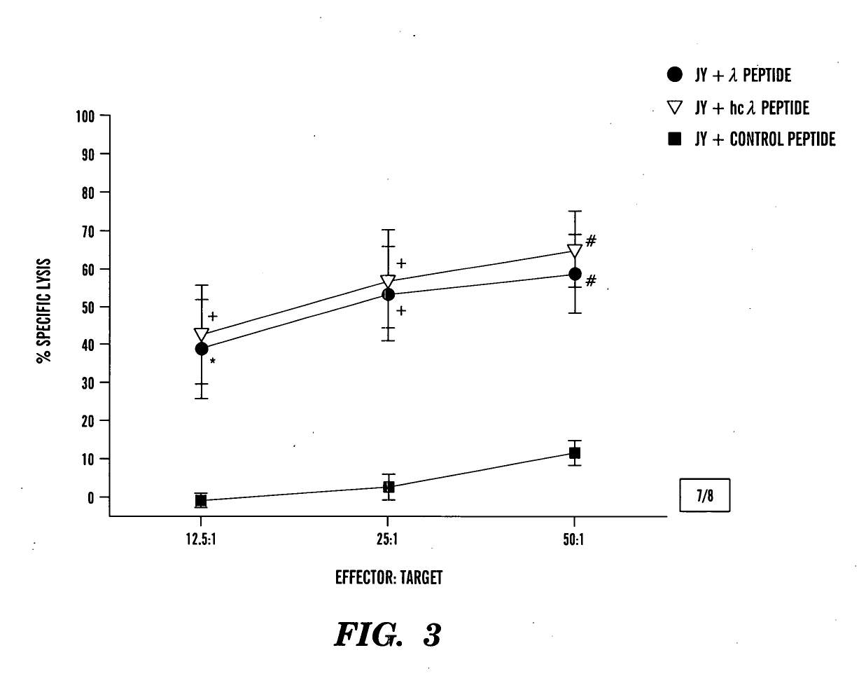

Induction of a peptide-mediated CTL response in vivo. To determine if these peptides were immunogenic as predicted by the algorithms, transgenic mice which express the human HLA-A2 allele were immunized with the native or heteroclitic peptides in an adjuvant relevant to clinical immunization schemes (Incomplete Freund's Adjuvant). The inventors discovered that a significant peptide-specific killer T cell activity was consistently induced (FIG. 2). These killer cells could lyse HLA-A2+ human B cell tumors (JY cells) coated with the native lambda 6 peptide, demonstrating that human Ig light chain-specific CTL can be induced in vivo and that lambda 6-derived peptides can be modified to increase MHC class I binding without significantly affecting T cell recognition of the native peptide expressed by target cells (FIG. 3).

FIG. 3 shows that the prediction of higher affinity binding of the heteroclitic peptide over the naive peptide results in increased number of peptide-specific T cells from the mouse and therefore increased killing of target containing or expressing cells. The alteration of the peptide increases its binding affinity should not affect the recognition of the peptide by the T cell. Therefore, any CTL activated using heteroclitic peptides should still recognize the normal peptide and be able to kill cells pulsed with the native peptide. This killing assay confirms the immunogenicity of the heteroclitic peptide, and confirms the cross-reactivity of T-cells from mice immunized with the heteroclitic peptide in killing target cells pulsed with the native peptide.

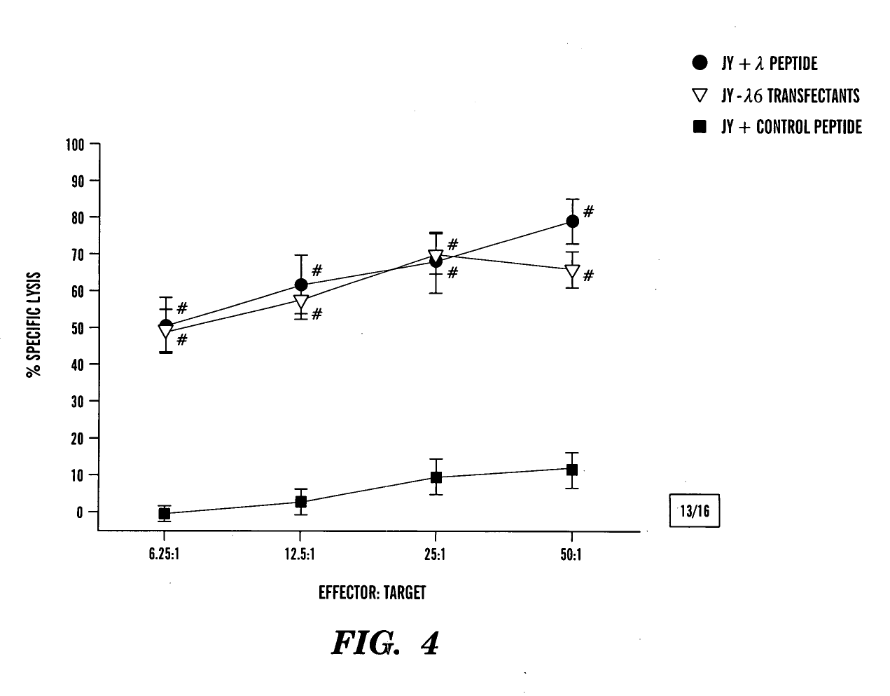

CTL induced in vivo with native lambda 6 (2-10) peptide also killed JY cells transfected with a gene encoding a full length, amyloidogenic lambda 6 light chain, demonstrating that neoplastic B cells process endogenous lambda 6 light chains and present sufficient levels of lambda 6-derived peptide(s) to enable T cell recognition and killing (FIG. 4). Since the transfected target cell must process the protein into peptides and load them onto the MHC Class I molecule, mimicking the in vivo processing which an amyloidogenic plasma cell would have to undergo for a peptide activated CTL to recognize them as targets, the inventors confirmed that CTL from mice immunized with a peptide can kill target cells which are making the protein from which the peptide is substantially similar to a region of.

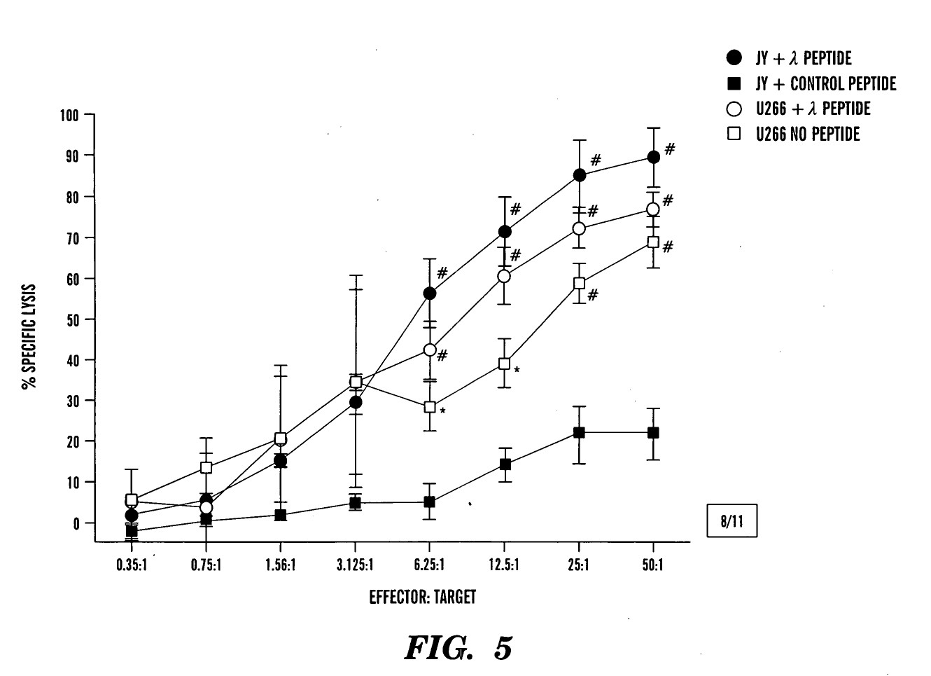

Finally, CTL induced with lambda 6 (2-10) peptide kill HLA-A2+, lambda 2 light chain-secreting U266 multiple myeloma plasma cells, suggesting that lambda 6 (2-10)-induced CTL cross-react with at least one other prevalent amyloidogenic light chain subtype, lambda 2, presumably by virtue of the presence of a shared T cell epitope in a conserved framework 1 region (Table 1) (FIG. 5). Note that all four of the most lambda light chains most common in AL amyloidosis share a common T cell epitope (Table 3), indicating that the inventors have discovered that the lambda 6 peptide may be applicable to subjects with lambda 1, 2, 3, or 6 light chain disease. FIG. 5 confirms the ability of the CTL elicited with the λ6 peptide immunization protocol to lyse U266 cells which naturally produce a protein of the λ2 sub-type of light chain. The λ2 peptide, which is most similar to the λ6 peptide, has a very low predicted binding affinity on HLA-A*0201. Killing of the U266 cells would seem to indicate that even at low frequency of peptide on the surface of the target, CTL can kill the target cell. Additionally, cross-reactivity of the CTL immunized with the λ6 peptide or other sub-types of light chain would make immunization effective for a higher percentage of AL amyloid patients.

TABLE 3
Shared amino acid sequences in AL amyloidogenic
light chain subtypes.

| Light | Most Common | | Percent AL |
|---|---|---|---|
| Chain | Framework 1 | | Amyloid |
| Subtype | Sequence | Sequence | Representation |
| λ6 | FMLTQPHSV | SEQ ID NO: 1 | 16% |
| λ1 | SVLTQPPSV | SEQ ID NO: 4 | 24% |
| λ2 | SELTQPASV | SEQ ID NO: 5 | 16% |
| λ3 | SELTQPPSV | SEQ ID NO: 6 | 11% |
| | Total | | 67% |

The amino acids in BOLD comprise the likely T cell epitope (T cell recognition sequence).

### Example 3

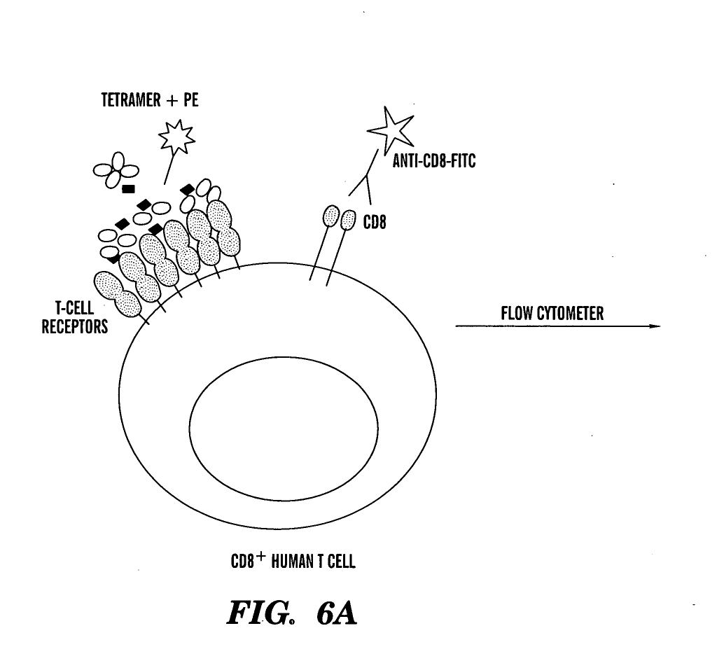

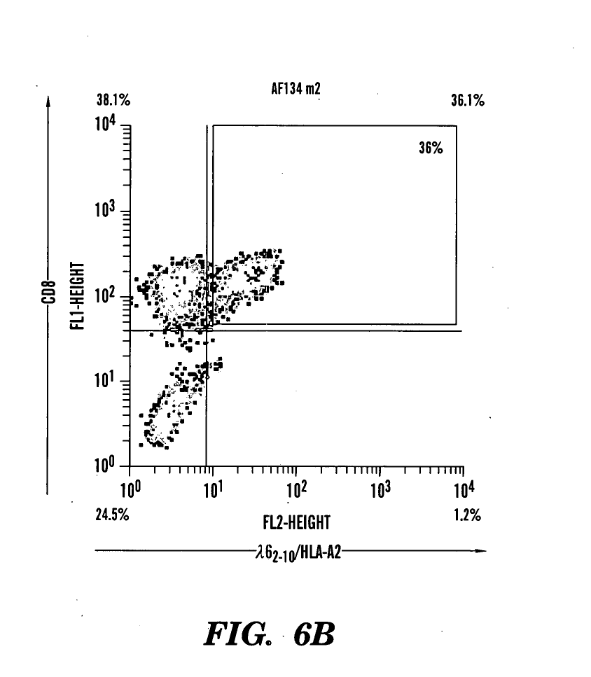

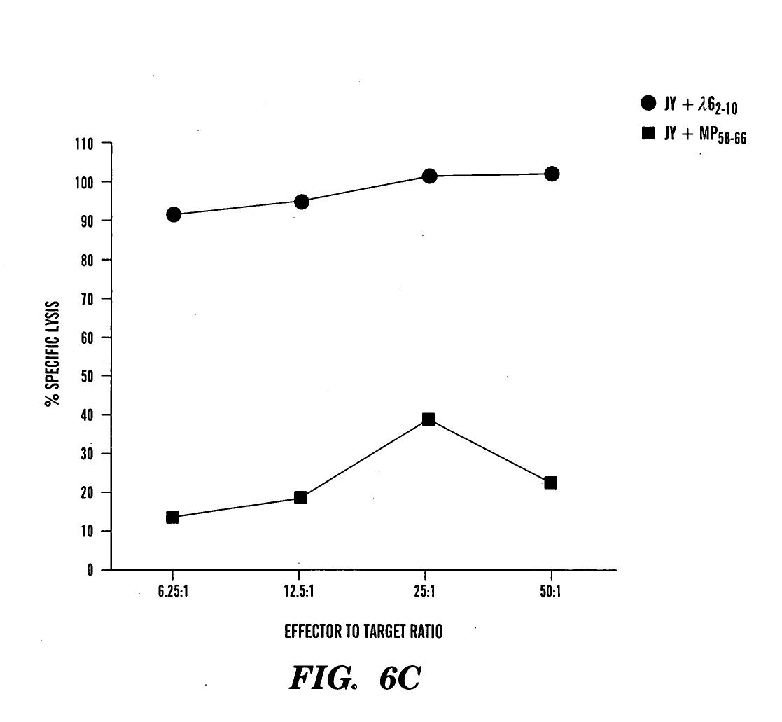

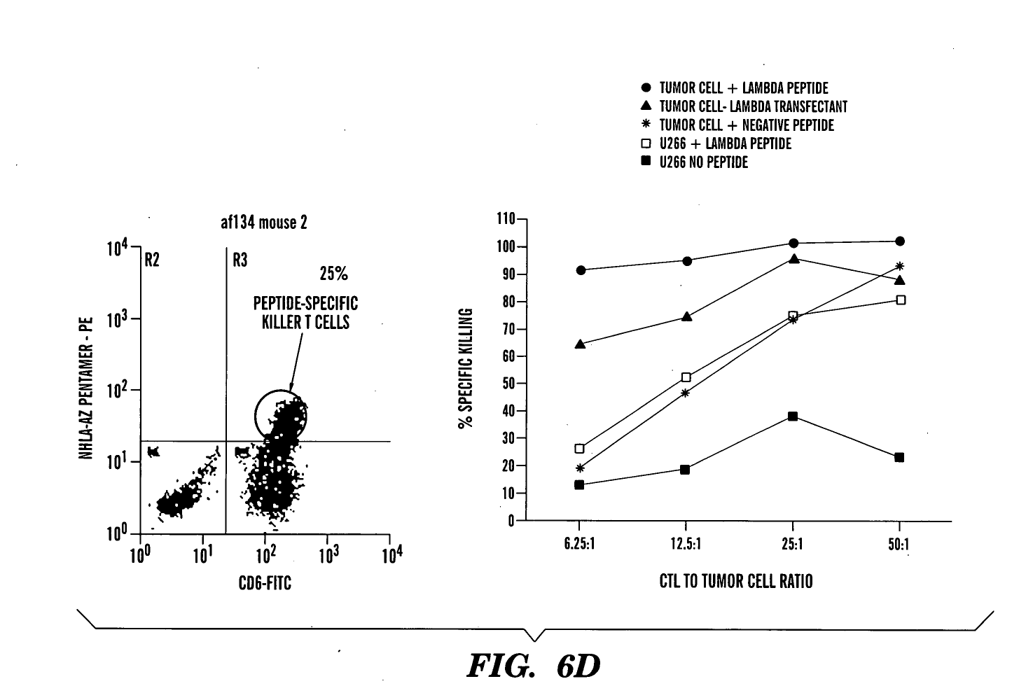

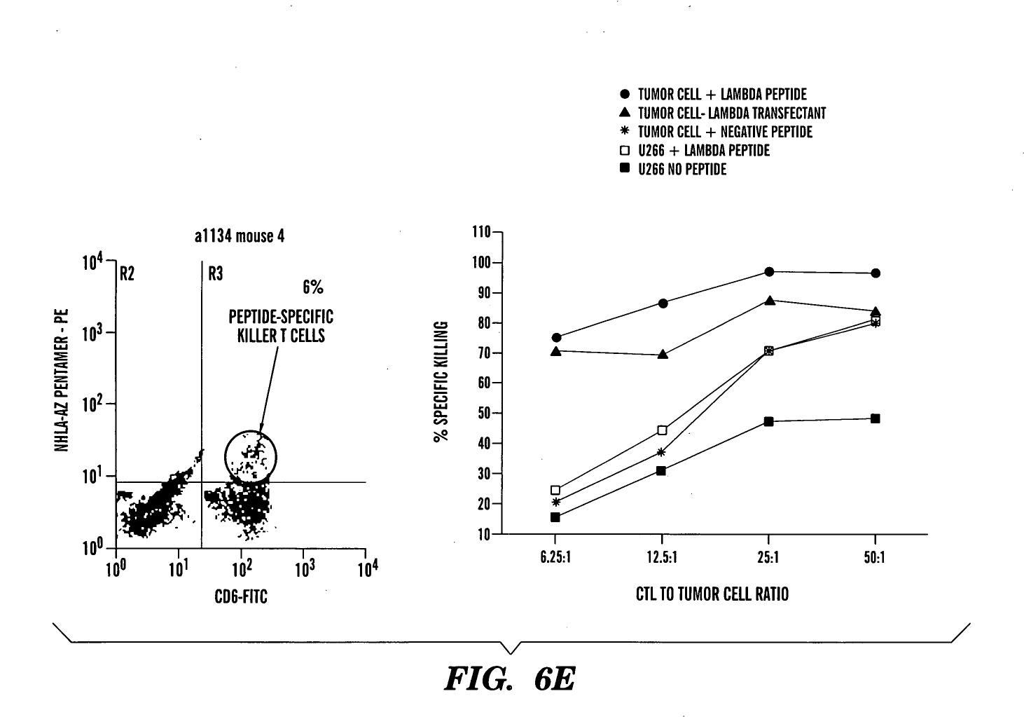

Identification of individual peptide-specific CTL in population of T cells. The inventors also discovered that the peptide/HLA-A*0201-specific T cell from mice immunized with λ6 peptide can be identified and isolated by flow cytochemistry. HHD mice immunized with λ6 peptide produced T cells which were stained with anti-CD8 (a CTL marker) FITC and with a PE labeled λ6 peptide/HLA-A*0201 pentamer. The cells were detected using Fluorescent Activated Cell Sorting (FACS) using a BD FASCcan Flow cytometer. Data shown in FIG. 6 shows representative 2 mice (D and E) of those that stained positively for pentamer, along with individual killing assays for those mice, and a summary of the data for the mouse of D also shown in B and C. As shown in FIG. 6, by identifying CTL that are specific for the λ6 peptide presented in HLA-A*0201, the inventors can isolate the T-cells and potentially increase the percentage of T cell that will kill the plasma cell or tumor cell targets. This technique would make for a highly efficient killing of the plasma cells and/or tumor cells, either in vitro or in vivo.

The inventors also quantitated the percentage of peptide-HLA-A2-specific CTL with fluorescent pentamers and demonstrated that the percentage of peptide-specific CTLs is highly variable, from ˜2% to over 15% of the CD8⁺ T cells.

### Example 4

Other peptides as a CTL target. The inventors have also discovered use of other peptides can elicit a CTL capable of killing immunoglobulin light chain-producing B or plasma cells. The inventors discovered peptides that are part of the protein Blimp-1, a highly expressed protein in plasma cells can be used as a second CTL target. Blimp-1 normally functions, and is necessary and sufficient, for plasma cell differentiation, in which is its required for functional plasma cell maintenance and antibody production in vivo. As similarly shown in Example 1, potentially immunogenic peptides of Blimp were identified in silico, and their MHC class I binding stability predicted using the three web-based algorithms. As shown in Table 4, a native mBlimp peptide, called “mBlimp₄₇₀₋₄₇₈” with peptide sequence SLFPRLYPV (SEQ ID NO:7) and a heteroclitic mBlimp peptide termed “heteroclitic mBlimp₄₇₀₋₄₇₈” with the peptide sequence YLFPRLYPV (SEQ ID NO:8) were analyzed using the NIH-BIMAS algorithm, and the inventors discovered the heteroclitic mBlimp₄₇₀₋₄₇₈ peptide determined to have a higher stability as compared to the native mBlimp₄₇₀₋₄₇₈ peptide.

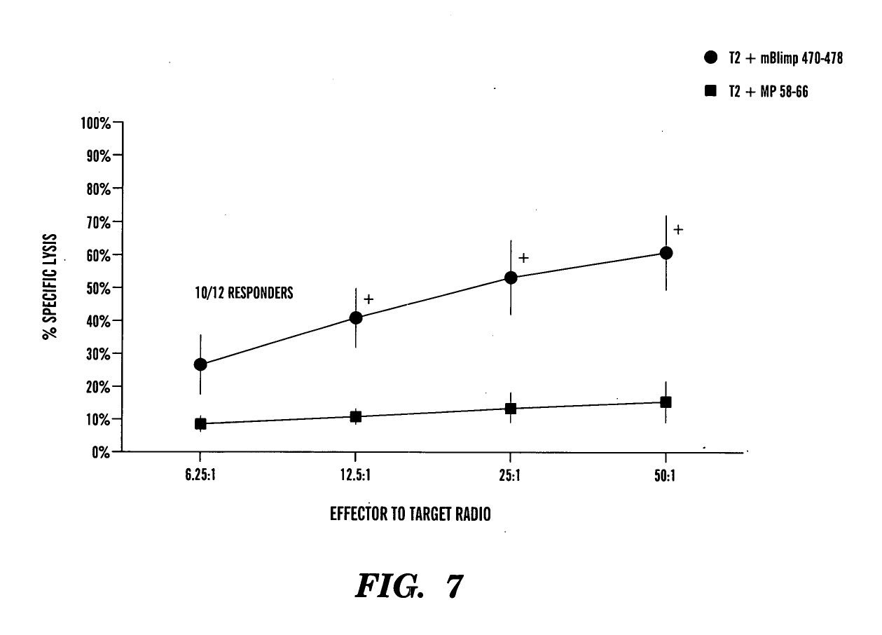

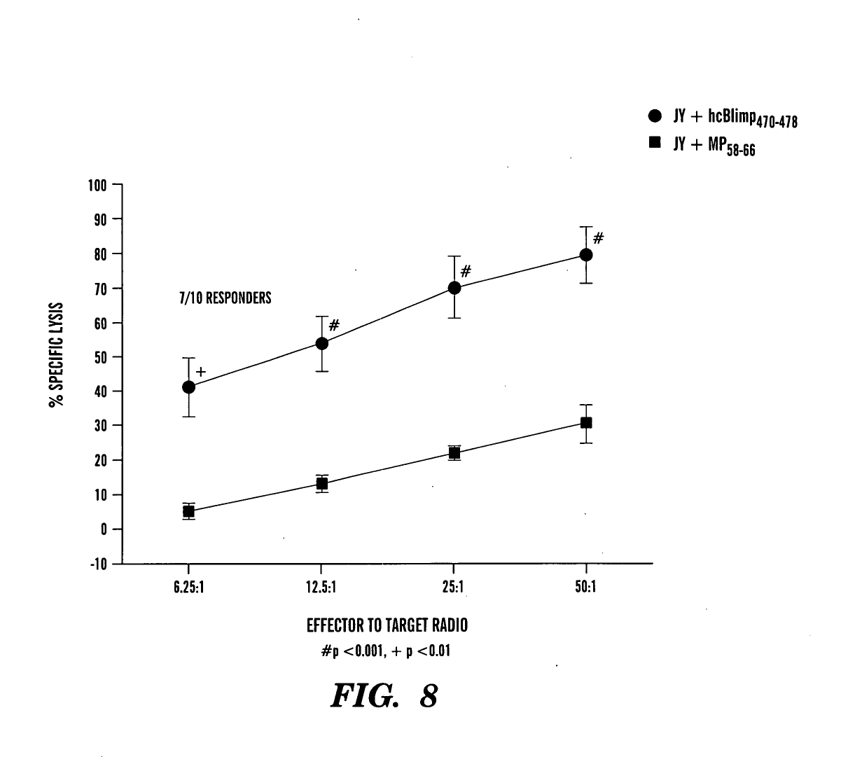

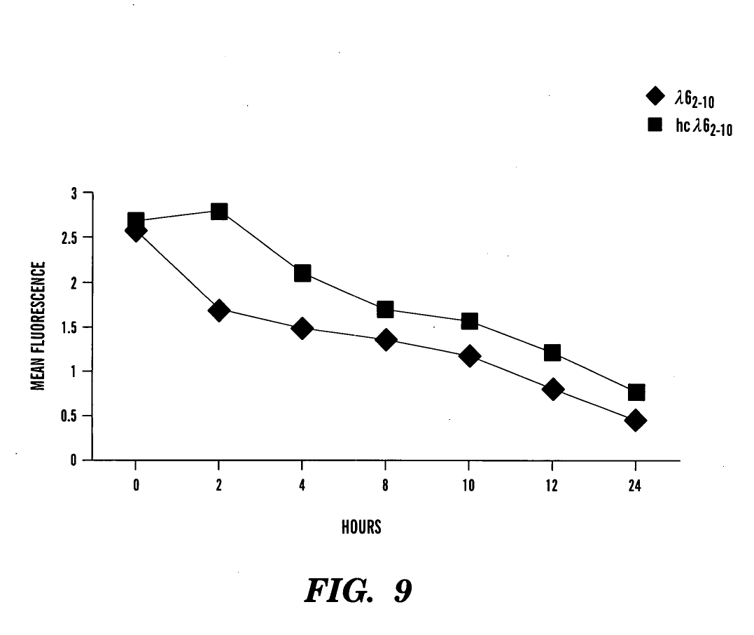

To determine if these mBlimp peptides were immunogenic as predicted by the algorithms, transgenic mice which express the human HLA-A2 allele were immunized with the native or heteroclitic peptides in an adjuvant relevant to clinical immunization schemes (Incomplete Freund's Adjuvant), as performed in Example 2. Significant peptide-specific killer T cell activity was consistently induced (FIG. 7 and FIG. 8). These killer cells could lyse HLA-A2+ human B cell tumors (JY cells) coated with the native mBlimp₄₇₀₋₄₇₈ and hc-mBlimp₄₇₀₋₄₇₈ peptide, demonstrating that human Ig light chain-specific CTL can be induced in vivo and that mBlimp₄₇₀₋₄₇₈-derived peptides can be modified to increase MHC class I binding without significantly affecting T cell recognition of the native peptide expressed by target cells.

TABLE 4
mBlimp peptide scores in a NIH-BIMAS peptide-MHC
Class I binding algorithm.

| Peptide | Amino Acid | NIH-BIMAS |
|---|---|---|
| Name | Sequence* | Score** |
| mBlimp₄₇₀₋₄₇₈ | SLFPRLYPV | 592 minutes |
| heteroclitic mBlimp₄₇₀₋₄₇₈ | YLFPRLYPV | 2723 minutes |

TABLE 5
A summary list of the immunogenic peptides
and corresponding SEQ ID NOS.

| | | SEQ |
|---|---|---|
| Name | PEPTIDE | ID |
| lambda 6(2-10) | FMLTQPHSV | 1 |
| heteroclitic lambda 6 | YMLTQPHSV | 2 |
| (2-10) (i) | | |
| heteroclitic lambda 6 | FLLTQPHSV | 3 |
| (2-10) (ii) | | |
| lambda 1 (2-10) | SVLTQPPSV | 4 |
| lambda 2 (2-10) | SELTQPASV | 5 |
| lambda 3 (2-10) | SELTQPPSV | 6 |
| mBlimp (470-478) | SLFRLYPV | 7 |
| heteroclitic mBlimp (470-478) | YLFRLYPV | 8 |
| HA[307-319] | PKYVKQNTLKLAT | 9 |
| PADRE | AKXVAAWTLKAAA | 10 |
| Malaria CS: T3 epitope | EKKIAKMEKASSVFNV | 11 |
| Hepatitis B surface antigen: | FFLLTRILTI | 12 |
| HBsAg[19-28] | | |
| Heat Shock Protein 65: hsp65 | DQSIGDLIAEAMDKVGNEG | 13 |
| [153-1] | | |
| bacille Calmette-Guerin | QVHFQPLPPAVVKL | 14 |
| Tetanus toxoid: TT[830-844] | QYIKANSKFIGITEL | 15 |
| Tetanus toxoid: TT[947-967] | FNNFTVSFWLRVPKVSASHLE | 16 |
| Tetanus toxoid: TT[947-967] | FNNFTVSFWLRVPKVSASHLE | 17 |

Collectively, these results support the feasibility of applying immunoglobulin light chain-derived peptide vaccines for AL amyloid immunotherapy and multiple myeloma. The pharmaceutical composition comprising the immunogenic peptide is appropriate for all HLA-A2+, lambda 6 AL amyloid subjects (about 10% of the AL amyloid population). Notably, the ability of CTL induced with the lambda 6 peptide to cross-react with cells producing lambda 1, 2, and 3 light chain subtypes, suggesting that vaccination with the lambda 6 peptide alone would be beneficial for up to 40% of all AL amyloid subjects and a large fraction of myleoma subjects, i.e. those expressing HLA-A2 and any of the light chain subtypes shown in Table 1.

The inventors discovered, using Ig light chain sequencing studies with bone marrow aspirates from 264 AL amyloid subjects the relative frequency of light chain subtypes in AL amyloidosis. Selecting the over-represented light chains in AL amyliod subjects, the inventors evaluated the affinity for formation of stable complexes for the HLA-A*0201 (HLA-A2)-binding peptides using three algorithms to predict the stability of peptide-MHC class I complexes. One such subtype (lambda 6; 16% of all AL amyloidosis cases) contains a conserved framework I sequence (lambda 6 (2-10)) predicted to bind stably to HLA-A2. The inventors confirmed this prediction using a standard fluorescence assay. Subsequently, the inventors tested the ability to induce CTL capable of lysing peptide-pulsed or Ig lambda 6-expressing human B cell tumors by immunizing HLA-A2 transgenic, (HHD) mice (Firat et al, 1999) with the lambda 6 (2-10) peptide. The investigators also tested the ability of lambda 6 (2-10) peptide-induced CTL to cross-react with plasma cells expressing a related lambda light chain subtype (lambda 2). Using public web-based algorithms we identified potential immunogenic peptides from a human immunoglobulin light chain (lambda 6) which causes 16-20% of all AL amyloidosis. Culture based assays supported the likelihood that the identified peptide would be immunogenic.

Using a peptide from A6 light chain as a target antigen, the inventors immunized HLA-A2 transgenic mice. Following immunization, the inventors restimulated splenocytes with peptide ex vivo and tested them for specificity in immunogenic lambda 6 peptide comprising chromium-release killing assays against a variety of cellular targets. The CTL kill HLA-A2 targets pulsed with cognate peptide or a closely related “heteroclitic” peptide, as well as HLA-A2 cell lines transfected with the full length lambda 6 protein. Using fluorescent pentamer to quantify the percentage of lambda 6 immunogenic peptide/HLA-A2-specific CTL, the inevntiods discovered that they could elicit a T cell response (CTL) between less than 2% to greater than 15% of the CD8⁺ T cell population.

Using a transgenic mouse line (from the Institut Pasteur, MTA attached) which expresses components of the human immune system, the inventors discovered that one natural peptide (lambda 6 (2-10)) and one slightly modified (one amino acid substitution-heteroclitic lambda 6 (2-10)) peptide from the human lambda 6 immunoglobulin light chain induce potent killer T cell responses in mice. These T cells kill human B cell tumors expressing the pathogenic lambda 6 immunoglobulin light chain. This indicates that approximately 8-10% of all amyloid subjects (those expressing the lambda 6 light chain and bearing the HLA-A*0201 allele) could benefit from the immunogenic peptide(s) as a therapy. The percentage of multiple myeloma subjects that would benefit has not been determined but is presumed to be on the same order as AL amyloid subjects, i.e. 16-20%. In addition, these T cells appear to recognize other amyloidogenic immunoglobulin light chains, suggesting that either the native or heteroclitic peptide could be useful for up to 40% of all AL amyloid or multiple myeloma subjects (i.e. those expressing lambda 1, 2, 3, or 6 light chains).

In its most basic form, the administration protocol would require only the synthesis of the 9mer immunogenic peptide and subcutaneous immunization 3 to 4 times with previously established or emerging experimental adjuvants. An administration protocol using the chosen peptide (lambda 6 Framework 1) plus an other antigen, for example a HBVc “helper” peptide (to elicit CD4⁺ T cell help), emulsified in Incomplete Freund's Adjuvant (IFA) was used. Subsequent to immunization, the spleens were harvested and splenocytes restimulated with peptide ex vivo.

Immunotherapy for AL amyloid is particularly attractive since the clonal plasma cell load is relatively low and the dysplastic plasma cell itself is relatively non-aggressive (McElroy et al, 1998). Furthermore, the fibrillogenic light chain itself is an obvious tumor-associated immunologic target that is highly expressed both by transformed AL amyloid plasma cells and by their clonal, post-germinal center B cell precursors (Manske et al, 2006). The lack of expression of the light chain subtype represented by the fibrillogenic protein by any normal cells other than a small fraction of B cells obviates concerns for pathologic autoimmune cross-reactivity with normal tissue. Furthermore, many immunotherapy trials which target a single tumor-associated peptide frequently demonstrate an initial rise in tumor immunity and a reduction in tumor load that is not durable because of antigen escape variants (Topalian et al, 1990). Emergence of an escape variant that does not produce the amyloidogenic light chain would be a desirable outcome. Even so, the approach also is applicable to multiple myeloma subjects whom have clonal plasma cells express lambda 6 (or other) light chain antibodies.

Treatment of plasma dyscrasias with immunogenic peptides would be relatively simple (off the shelf) and cheap and would be applicable to several plasma cell dyscrasias (AL amyloid, multiple myeloma, MGUS, Waldenstrom's macroglobulinemia). Unlike chemotherapy, significant involvement of multiple organs with amyloid fibrils would not preclude immunotherapy nor would prior chemotherapy. No to minimal toxicity is anticipated since the only other cells expressing the target lambda 6 protein would be an extremely minor population of normal B cells, and would expected to have no clinical consequences. Although the lambda 6 native and heteroclitic peptides are most applicable at present only to a subset of AL amyloid subjects (i.e. those expressing HLA-A2 and producing lambda 6 light chains, or approximately 8-10% of all AL amyloid subjects), the inventors have discovered that the responses to the immunogenic peptides based on lambda 6 also cross-react with lambda 1, 2, and 3 light chains, indicating that the application of these immunogenic peptides corresponding to SEQ ID NOS: 1 to 6 would be applicable to as much as 40% of the AL amyloid population and a similar subset of multiple myeloma subjects. Furthermore, second generation peptides derived with the same strategy would target virtually all types of AL amyloid and the other plasma cell dyscrasias.

### REFERENCES

The references cited herein and throughout the application are incorporated herein by reference.
1. Falk R, Skinner M. The systemic amyloidoses: An overview. Adv Int Med. 2000; 45:107-137.
2. Obici L, Perfetti V, Palladini C, Moratti R, Merlini C. Clinical aspects of systemic amyloid diseases. Biochim Biophys Acta. 2005; 1753:1 1-22.
3. Trinkaus-Randall V, Walsh M T, Steeves 5, Monis G, Connors L H, Skinner M. Cellular response of cardiac fibroblasts to amyloidogenic light chains. Am J. Pathol. 2005; 166:197-208.
4. Witzig T E, Meyers C, Themeau T, Greipp P R. A prospective study of CD38/45 flow cytometry and immunofluorescence microscopy to detect blood plasma cells in subjects with plasma cell proliferative disorders. Leuk Lymphoma. 2000; 38:345-350.
5. McElroy E, Witzig T, Gertz M, Greipp P, Kyle R. Detection of monoclonal plasma cells in the peripheral blood of subjects with primary amyloidosis. Br J. Haematol. 1998; 100:326-327.
6. Perfetti V, Ubbiali P, Magni M, et al. Cells with clonal light chains are present in peripheral blood at diagnosis and in apheretic stem cell harvests of primary amyloidosis. Bone Marrow Transplant. 1999; 23:323-327.
7. Kumar 5, Rajkumar S V, Kyle R A, et al. Prognostic value of circulating plasma cells in monoclonal gammopathy of undetermined significance. J Clin Oncol. 2005; 23:5668-5674.
8. Nowakowski C S, Witzig T E, Dingli D, et al. Circulating plasma cells detected by flow cytometry as a predictor of survival in 302 subjects with newly diagnosed multiple myeloma. Blood. 2005; 106:2276-2279.
9. Kyle R, Gertz M, Greipp P, et al. A trial of three regimens for primary amyloidosis: colchicine alone, melphalan and prednisone, and melphalan, prednisone, and colchicine. NEng J Med. 1997; 336:1202-1207.
10. Skinner M, Sanchorawala V, Seldin D C, et al. High-dose melphalan and autologous stem-cell transplantation in subjects with AL amyloidosis: an 8-year study. Ann Intern Med. 2004; 140:85-93.
11. Dispenzieri A, Kyle R A, Lacy M Q, et al. Superior survival in primary systemic amyloidosis subjects undergoing peripheral blood stem cell transplantation: a case-control study. Blood. 2004; 103:3960-3963.
12. Dispenzieri A, Lacy M Q, Rajkumar S V, et al. Poor tolerance to high doses of thalidomide in subjects with primary systemic amyloidosis. Amyloid. 2003; 10:257-261.
13. Dispenzieri A, Gertz M A, Lacy M Q, et al. A phase II trial of imatinib in subjects with refractory/relapsed multiple myeloma. Leuk Lymphoma. 2006; 47:39-42.
14. O'Nuallain B, Hrncic R, Wall J S, Weiss D T, Solomon A. Diagnostic and therapeutic potential of amyloidreactive IgG antibodies contained in human sera. J. Immunol. 2006; 176:7071-7078.
15. Solomon A, Weiss D T, Wall J S. Immunotherapy in systemic primary (AL) amyloidosis using amyloid-reactive monoclonal antibodies. Cancer Biother Radiopharm. 2003; 18:853-860.
16. Maecker B, Sherr D H, Vonderheide R H, et al. The shared tumor-associated antigen cytochrome P450 1 BI is recognized by specific cytotoxic T cells. Blood. 2003; 102:3287-3294.
17. Maecker B, von Bergwelt-Baildon M S, Sherr D H, Nadler L M, Schultze J L. Identification of a new HLA-A*0201-restricted cryptic epitope from CYPIBI. Int J. Cancer. 2005; 115:333-336.
18. Rodeberg D A, Nuss R A, Elsawa S F, Celis E. Recognition of six-transmembrane epithelial antigen of the prostate-expressing tumor cells by peptide antigen-induced cytotoxic T lymphocytes. Clin Cancer Res. 2005; 1 I:4545-4552.
19. Alters S E, Gadea J R, Sorich M, O'Donoghue C, Talib 5, Philip R. Dendritic cells pulsed with CEA peptide induce CEA-specific CTL with restricted TCR repertoire. J. Immunother. 1998; 21:17-26.
20. Wobser M, Keikavoussi P, Kunzmann V, Weininger M, Andersen M H, Becker J C. Complete remission of liver metastasis of pancreatic cancer under vaccination with a HLA-A2 restricted peptide substantially similar to a region of the universal tumor antigen survivin. Cancer Immunol Immunother. 2006; 55:1294-1298.
21. Knutson K L, Schiffman K, Cheever M A, Disis M L. Immunization of cancer subjects with a HER-2/neu, HLA-A2 peptide, p 369-377, results in short-lived peptide-specific immunity. Clin Cancer Res. 2002; 8:1014-1018.
22. Scardino A, Gross D A, Alves P, et al. HER-2/neu and hTERT cryptic epitopes as novel targets for broad spectrum tumor immunotherapy. J. Immunol. 2002; 168:5900-5906.
23. Trojan A, Witzens M, Schultze J L, et al. Generation of cytotoxic T lymphocytes against native and altered peptides of human leukocyte antigen˜A*0201 restricted epitopes from the human epithelial cell adhesion molecule. Cancer Res. 2001 61:4761-4765.
24. Rodeberg D A, Nuss R A, Elsawa S F, Erskine C L, Celis E. Generation of tumoricidal PAX3 peptide antigen specific cytotoxic T lymphocytes. Int J. Cancer. 2006; 1 19:126-132.
25. Vonderheide R H, Hahn W C, Schultze J L, Nadler L M. The telomerase catalytic subunit is a widely expressed tumor-associated antigen recognized by cytotoxic T lymphocytes. Immunity. 1999; 10:673-679.
26. Trojan A, Schultze J L, Witzens M, et al. Immunoglobulin framework-derived peptides function as cytotoxic Tcell epitopes commonly expressed in B-cell malignancies. Nat. Med. 2000; 6:667-672.
27. Molldrem J, Lee P, Wang C, et al. Evidence that specific T lymphocytes may participate in the elimination of chronic myelogenous leukemia. Nat. Med. 2000; 6:1018-1 023.
28. Manske M K, Zuckerman N S, Timm M M, et al. Quantitative analysis of clonal bone marrow CDI 9+B cells: use of B cell lineage trees to delineate their role in the pathogenesis of light chain amyloidosis. Clin Immunol. 2006; 120:106-120.
29. Topalian S L, Kasid A, Rosenberg S A. Immunoselection of a human melanoma resistant to specific lysis by autologous tumor-infiltrating lymphocytes. Possible mechanisms for immunotherapeutic failures. J. Immunol. 1990; 144:4487-4495.
30. Firat H, Garcia-Pons F, Tourdot 5, et al. H-2 class I knockout, HLA-A2.1-transgenic mice: a versatile animal model for preclinical evaluation of antitumor immunotherapeutic strategies. Eur J. Immunol. 1999; 29:3112-3121.

1. A method for inducing a CD8+ cytotoxic lymphocyte cell (CTL) response in a subject comprising administering to the subject a pharmaceutical composition comprising an immunogenic peptide, wherein the immunogenic peptide is a region of the light chain of an immunoglobulin or a fragment or derivative thereof, and wherein the pharmaceutical composition does not contain a heavy chain of the immunoglobulin or a fragment thereof, and wherein the immunoglobulin is expressed by a malignant cell.

2. The method of claim 1, for treating a plasma cell dyscrasias, disorder or malignancy.

3. A pharmaceutical composition of claim 1 for treating a plasma cell dyscrasias, disorder or malignancy.

4. The method of claim 1, wherein the region of the immunoglobulin light chain is the variable region of the immunoglobulin light chain, wherein the region does not contain an immunoglobulin heavy chain.

5. The method of claim 4, wherein the variable region of the immunoglobulin light chain is a lambda (λ) variable region of the immunoglobulin light chain or a variant thereof.

6. The method of claim 4, wherein the variable region of the immunoglobulin light chain is a kappa (κ) variable region of the immunoglobulin light chain or a variant thereof.

7. The method of claim 5, wherein the lambda variable region is selected from the group consisting: lambda 1, 2, 3, 4, 5, 6, 7, 8, 9 or 10 light chain.

8. The method of claim 5, wherein the lambda variable region is lambda 6 light chain, or a derivative or fragment thereof.

9. The method of claim 5, wherein the lambda variable region is lambda 2 light chain, or a derivative or fragment thereof.

10. The method of claim 1, wherein the immunogenic peptide is between 5-9 residues.

11. The method of claim 1, wherein the immunogenic peptide is between 9-20 residues.

12. The method of claim 1, wherein the immunogenic peptide is greater than 20 residues.

13. The method of claim 1, wherein the immunogenic peptide is 9 residues.

14. The method of claim 8, wherein a region of lambda 6 is lambda 6 (2-10), or a derivative or fragment thereof.

15. The method of claim 14, wherein the immunogenic peptide is lambda 6 (2-10) and has an amino acid sequence FMLTQPHSV (SEQ ID NO: 1), or a derivative or fragment thereof.

16. The method of claim 15, wherein the derivative of lambda 6(2-10) is a heteroclitic immunogenic peptide of lambda 6, or a derivative or fragment thereof.

17. The method of claim 16, wherein the heteroclitic immunogenic peptide of lambda 6 has the amino acid sequence FLLTQPHSV (SEQ ID NO: 3), or a derivative or fragment thereof.

18. The method of claim 1, wherein the malignant cell is a plasma cell.

19. The method of claim 18, wherein the plasma cell is associated with a plasma cell disorder or plasma cell dyscrasias.

20. The method of claim 1, wherein the malignant cell is a cancer cell.

21. The method of claim 1, where in the pharmaceutical composition further comprises at least one other antigen or carrier peptide.

22. The method of claim 21, where in the antigen is a helper peptide.

23. The method of claim 21, wherein the antigen induces CD4⁺ T cell response.

24. The method of claim 22, wherein the helper peptide is HBVc peptide.

25. The method of claim 1, where in the pharmaceutical composition further comprises an adjuvant.

26. The method of claim 25, wherein the adjuvant is selected from a group consisting essentially of: complete Freunds adjuvant (CFA), incomplete Freunds adjuvant (IFA), QS21, aluminium hydroxide gel, MF59 and calcium phosphate.

27. The method of claim 21, wherein the antigen or carrier peptide is associated with the immunogenic peptide.

28. The method of claim 27, wherein the association is via covalent bond or non-covalent bond.

29. The method of claim 28, wherein covalent bond is a peptide bond.

30. The method of claim 21, wherein the carrier peptide is selected from the group consisting of; SEQ ID NOs: 9 to SEQ ID NO:17 or derivatives, analogues or fragments thereof.

31. A method for treating or preventing a plasma cell disorder or malignancy in a subject, the method comprising administering to the subject an effective amount of a immunogenic peptide of claim 1, wherein the immunogenic peptide generates an immune response to the immunoglobulin producing plasma cell thereby preventing or reducing the symptoms of the plasma cell disorder or malignancy.

32. The method of claims 30 or 31, wherein the plasma cell disorder or malignancy is plasma cell dyscrasias.

33. The method of claim 32, wherein the plasma cell dyscrasias selected from a group consisting of: AL amyloidosis, multiple myeloma, monoclonal gammopathies, monoclonal gammopathies of undetertermined significance (MGUS), and Waldnestrom's macroglobulinemia, or combinations thereof.

34. The method of claim 33, wherein the plasma cell dyscrasias is primary amyloidosis.

35. The method of claim 33, wherein the plasma cell dyscrasias is secondary amyloidosis.

36. The method of claim 34, wherein the primary amyloidosis is AL amyloidosis.

37. The method of claim 33, wherein the plasma cell dyscrasias is multiple myeloma.

38. The method of claims 1 or 31, wherein the subject is a mammal.

39. The method of claim 38, wherein the subject is human.

40. The method of claims 1 or 31, wherein the subject is at risk of developing a plasma cell disorder or malignancy.

41. The method of claims 1 or 31, wherein administration is intraperitoneally, orally, subcutaneously, intramuscularly, intranasally, topically or intravenously.

42. The method of claim 31, wherein the immunogenic peptide is administered as a pharmaceutical composition.

43. The method of claims 1 or 31, wherein the subject is administered other therapeutic agents before, at the same time as, or after administration of the immunogenic peptide.

44. A method for designing an immunogenic peptide for the treatment or prevention a disease or malignancy in a subject, wherein the disease or malignancy is associated with a malignant cell, the method comprising:
(a) identifying proteins expressed at a higher level by the malignant cells from the subject with a disease or disorder as compared to the level expressed by non-malignant cells or cells in a normal subject;
(b) selecting a region of the protein identified in step (a) and screening the amino acid sequence of such selected region, or an amino acid sequence of substantial similarity thereof, for predicted stability to complex with MHC Class I molecules;
wherein the amino acid sequence that is predicted to form stable complex with MHC Class I is used as an immunogenic peptide for the treatment or prevention of a disease or malignancy.

45. The method of claim 44, further comprising a step of administering the immunogenic peptide to a rodent to elicit an immune response, and assessing the effect of the immune response to kill cells comprising the protein, or a fragment thereof, which was identified in step (a).

46. The method of claim 44, wherein the malignant cell is a cancer cell.

47. The method of claim 44, wherein the malignant cell is a plasma cell.

48. The method of claim 44, wherein the disease or malignancy is a plasma cell disorder or plasma cell dyscrasias.

49. The method of claim 44, wherein the disease or malignancy is an amyloidogenic disease.

50. The method of claim 44, wherein the disease or malignancy is multiple myeloma.

51. The method of claim 49, wherein the amyloidogenic disease is AL amyloidosis.

52. The method of claim 49, wherein the amyliodogenic disease is an amyloid-related disease.

53. The method of claim 52, wherein the amyloid-related disease is Alzheimer's disease, Down's syndrome, vascular dementia or cognitive impairment, type II diabetes mellitus, amyloid A (reactive), secondary amyloidosis, familial mediterranean fever, familial nephrology with urtcaria and deafness (Muckle-wells Syndrome), amyloid lambda L-chain or amyloid kappa L-chain (idiopathic, multiple myeloma or macroglobulinemia-associated) A beta 2M (chronic hemodialysis), ATTR (familial amyloid polyneuropathy (Portuguese, Japanese, Swedish), familial amyloid cardiomyopathy (Danish), isolated cardiac amyloid, (systemic senile amyloidosis) AIAPP or amylin insulinoma, atrial naturetic factor (isolated atrial amyloid), procalcitonin (medullary carcinoma of the thyroid), gelsolin (familial amyloidosis (Finnish), cyctatin C (heritiaty cerebral hemorrhage with amyloidosis (Icelandic), AApo-A-I (familial amyloidotic polyneuropathy—Iowa), AApo-A-II (accelerated senescence in mice), fibrinogen-associated amyloid; and Asor or Pr P-27 (scrapie, Creutzfeld jacob disease, Gertsmann-Straussler-Scheinker syndrome, bovine spongiform encephalitis) and subjects who are homozygous for the apolipoprotein E4 allele.

54. A method for producing an anti-idiotype antibody in a mammal, the method comprising administering to the mammal an antigenic amount of the immunogenic peptide as defined by any of the claims 4-17, wherein the immunogenic peptide elicits the production of antibodies having specificity towards a polypeptide comprising the immunogenic peptide or a fragment or derivative thereof, or the immunogenic peptide fragment itself.

55. The method according to claim 54, wherein the anti-idiotype antibody is specific to the malignant cell which expresses the immunoglobulin from which the peptide fragment is derived.

56. The method according to claim 54, which further comprises the step of generating hybridoma cells by somatic cell hybridization for the production of monoclonal or polyclonal antibodies.
